(19) United States
(12) Patent Application Publication
(10) Pub. No.: US 2021/0322396 A1
(43) Pub. Date: Oct. 21, 2021

(54) COMPOSITIONS WITH SYNERGISTIC PERMEATION ENHANCERS FOR DRUG DELIVERY

(71) Applicant: Children's Medical Center Corporation, Boston, MA (US)

(72) Inventors: Daniel S. Kohane, Newton, MA (US); Rong Yang, Cambridge, MA (US)

(73) Assignee: Children's Medical Center Corporation, Boston, MA (US)

(21) Appl. No.: 17/272,212

(22) PCT Filed: Aug. 30, 2019
(86) PCT No.: PCT/US2019/049084

§ 371 Date: Feb. 26, 2021

Related U.S. Application Data
(60) Provisional application No. 62/814,161, filed on Mar. 5, 2019.
(60) Provisional application No. 62/726,058, filed on Aug. 31, 2018.

(51) Int. Cl. A61K 31/445; A61K 47/20; A61K 47/06; A61K 47/34; A61K 31/529; A61K 31/496; A61K 31/573; A61K 45/06; A61M 31/00; A61M 25/00; A61K 9/00

(52) U.S. Cl. CPC A61K 31/445; A61K 47/20; A61K 47/06; A61K 47/34; A61K 31/529; A61M 2210/0662; A61K 31/573; A61K 45/06; A61M 31/00; A61M 25/0021; A61K 9/0046; A61K 31/496

(57) ABSTRACT

The present disclosure provides compositions and methods for delivery of therapeutic agents across a barrier. The compositions include a therapeutic agent (e.g., antimicrobial agent, antibiotic, or anesthetic agent), a permeation enhancer which increases the flux of the therapeutic agent across the barrier, and a matrix forming agent, wherein the composition comprises between about 0.5-5.0% wt/vol of a permeation enhancer that is sodium dodecyl sulfate; wherein the compositions comprise between about 0.5-2.5% wt/vol of a permeation enhancer that is bupivacaine; wherein the compositions comprise between about 1.5-12.0% wt/vol of a permeation enhancer that is limonene; and wherein the compositions comprise between about 9.0-19.0% wt/vol of a polymer that is poloxamer 407-poly(butoxy)phosphoester; and optionally further comprises between about 0.01-0.50% wt/vol of another therapeutic agent that is a sodium channel blocker anesthetic agent (e.g., tetrodotoxin).

### RELATED APPLICATIONS

This application claims priority under 35 U.S.C. § 119(e) to U.S. Provisional Application, U.S. Ser. No. 62/726,058, filed Aug. 31, 2018, and U.S. Ser. No. 62/814,161, filed Mar. 5, 2019, each of which is incorporated herein by reference.

### GOVERNMENT SUPPORT

This invention was made with government support under grants DC015050 and DC016644 awarded by the National Institutes of Health. The government has certain rights in the invention.

### BACKGROUND

Twelve to 16 million physician visits per year in the United States are attributed to otitis media (OM), making it the most common specifically treated childhood disease. [(a) Berman, S., Otitis media in children. N Engl J Med 1995, 332, 1560-5; (b) Fried, V. M.; Makuc, D. M.; Rooks, R. N. Ambulatory health care visits by children: principal diagnosis and place of visit.; 137; Washington, D.C.: Government Printing Office, 1998.: 1998.]. Acute OM (AOM) has a prevalence of 90% within the first 5 years of life, [Teele, D. W.; Klein, J. O.; Rosner, B., Epidemiology of otitis media during the first seven years of life in children in greater Boston: a prospective, cohort study. The Journal of infectious diseases 1989, 160 (1), 83-94] and 90-95% of all U.S. children have at least one documented middle ear effusion by age 2. [Casselbrant, M. L.; Mandel, E. M., Epidemiology. In Evidence-based otitis media, Rosenfeld, R. M.; Bluestone, C. D., Eds. Decker, Inc.: Hamilton, British Columbia, 1999; pp 117-137]. 25% percent of all prescriptions written for children are for treatment of acute otitis media. Recurrence of the disease is also striking, with one third of all children in the U.S. having 6 or more episodes of AOM by age 7. [Faden, H.; Duffy, L.; Boeve, M., Otitis media: back to basics. The Pediatric infectious disease journal 1998, 17 (12), 1105-12; quiz 1112-3]. Moreover, epidemiological studies suggest that the prevalence of recurrent OM among children, particularly infants, is on the rise. [Lanphear, B. P.; Byrd, R. S.; Auinger, P.; Hall, C. B., Increasing prevalence of recurrent otitis media among children in the United States. Pediatrics 1997, 99 (3), E1]. The incidence of OM in children of other industrialized nations is similar to that in the U.S. In the developing world, OM remains a significant cause of childhood mortality due to the development of chronic suppurative otitis media which frequently results in permanent hearing sequelae, and due to intracranial complications estimated to result in more than 25,000 deaths worldwide. [Acuin, J. Otitis Media: Burden of Illness and Management Options; World Health Organization: Geneva, Switzerland, 2004].

Acute OM is the most common reason for antimicrobial prescribing in U.S. children and due to the high prevalence of disease and frequent recurrences is believed to be partially responsible for the ongoing increase in antibiotic resistance among pathogenic bacteria. Despite the success in reducing antimicrobial use in children by approximately 25% over the past decade, the increase in antimicrobial resistance has continued. Additionally, Acute Otitis Media (AOM) is one of the most common childhood diseases, accounting for over 20 million physician visits each year in the U.S.^{1,2}. Recurrence is also common, with one third of children having six or more episodes of AOM by the age of seven 3. Up to 80% of children with AOM have mild to severe pain during the onset of the infection, of which about 40% have severe pain^{4 5}. The first 24 to 48 hours are considered to be the most painful period of AOM but about 30% of the children have pain for 3-7 days⁶. Consequently, many AOM guidelines recommend the use of analgesics as an essential part of the treatment⁷. AOM commonly causes pain and distress in children. Existing analgesic ototopical drops have limited effectiveness due to the impermeable nature of the tympanic membrane. Oral analgesic medications are commonly used⁸, although it is not clear that they are helpful⁹. The effectiveness of commercial ototopical products in AOM is also questionable^{10,11}. Nonetheless, local topical treatment of pain in AOM remains desirable since side effects from systemic drug distribution would be avoided, the pain relief could be faster in onset, be more intense, and last longer than with oral analgesia.

Present treatment of ear infections consists of systemic oral antibiotics, a treatment which requires multiple doses over 5-10 days and systemic exposure to antibiotics. The rise in antibiotic resistance, coupled with the many multifactorial etiology of OM pose difficulties in diagnosis and treatment of OM. Furthermore, current treatment presents a number of drawbacks including patient compliance issues due to gastrointestinal side effects, lack of an effective concentration of drug at the site of infection, and the potential for opportunistic infections. Even after acute signs of infection subside, generally within 72 hours, the root cause of the infection may persist for the remainder of the treatment, and beyond, even up to 2 months. Thus, making compliance with a physician's prescription important to prevent reoccurrence of infection.

Local, sustained delivery of active therapeutics directly to the middle ear for the treatment of OM could allow for much higher concentrations of the drug in the middle ear than from systemic administration, while minimizing systemic exposure and its adverse effects. However, the tympanic membrane (TM), while only 10 cell-layers thick, presents a barrier that is largely impermeable to all but the smallest, moderately hydrophobic molecules. Despite being the thinnest layer of skin, it is still a barrier to trans-tympanic membrane diffusion. Therefore, the direct treatment of middle ear infections is problematic. The shortcomings of the current treatment of ear diseases, such as middle ear infections, suggest the need for a new treatment which is noninvasive and direct acting. Additionally, local topical treatment of pain associated with AOM is also desirable.

### SUMMARY

Provided herein are compositions and methods aimed at non-invasive trans-tympanic otitis media (OM) treatment with sustained drug flux across the tympanic membrane (TM). Chemical permeation enhancers (CPEs), commonly employed for trans-dermal delivery, can enable such a trans-tympanic flux. In certain embodiments, a single application of an optimized formulation could provide high concentrations of antibiotics localized to the middle ear, resulting in eradication of bacterial otitis media without the drawbacks of oral therapy. Such formulations may also useful in the treatment of other diseases of the ear requiring drug delivery across the tympanic membrane.

Typical OM treatments consist of a 10-day course of broad spectrum oral antibiotics. The widespread use of systemic antibiotics against a disease of such high prevalence and recurrence is believed to be partially responsible for the ongoing increase in antibiotic resistance seen in pathogenic bacteria in the nasopharynx. In most cases, antibiotic-resistant infections like pneumonia, skin, soft tissue, and gastrointestinal infections require prolonged and/or costlier treatments, extend hospital stays, necessitate additional doctor visits and healthcare use, and result in greater disability and death compared with infections that are easily treatable with antibiotics. Compliance with multi-dose regimens can also be difficult in some parts of the world. Compliance and antibiotic resistance may also be more problematic in the long-term prophylaxis of recurrent OM. An effective sustained local therapy could address the issue of compliance, affect the development of drug-resistant and chronic suppurative otitis media, and reduce the need for tympanostomy tube placement (devices implanted in the TM to enhance middle ear drainage in recurrent OM). [Khoo, X.; Simons, E.; Chiang, H.; Hickey, J.; Sabharwal, V.; Pelton, S.; Rosowski, J.; Langer, R.; Kohane, D., Formulations for trans-tympanic antibiotic delivery. Biomaterials 2013, 34, 1281-8].

The TM is a tri-layer membrane whose outer layer is a stratified squamous keratinizing epithelium continuous with the skin of the external auditory canal. The inner-most layer is a simple cuboidal mucosal epithelium. Between these epithelia is a layer of fibro-elastic connective tissue and associated blood vessels and nerves. The human TM is only about 100 m thick, but the 6-10 cell layer outer epithelium forms an impenetrable barrier against all but the smallest lipophilic molecules due to its keratin- and lipid-rich stratum corneum. [Doyle, W. J.; Alper, C. M.; Seroky, J. T.; Karnavas, W. J., Exchange rates of gases across the tympanic membrane in rhesus monkeys. Acta oto-laryngologica 1998, 118 (4), 567-73].

Localized, sustained drug delivery directly to target tissues has several advantages over systemic application, including fewer adverse systemic effects, smaller quantities of drug used, potentially better therapeutic outcomes, and reduced costs. The impermeability of the TM is a central challenge for the development of local therapies.

Chemical permeation enhancers (CPEs) are used to safely increase small molecule flux in transdermal drug delivery. Several are FDA approved for use in humans. These agents are often surfactants, comprising a heterogeneous group of amphiphilic organic molecules with hydrophilic heads and hydrophobic tails. Several classes of surfactants have been studied. Surfactants reversibly modify lipids by adsorption at interfaces and removal of water-soluble agents that act as plasticizers. Cationic surfactants are known to produce greater increases in permeant flux than anionic surfactants, which in turn increase permeability more than nonionic surfactants. A broad range of non-surfactant chemical enhancers (e.g., terpenes) has also been used with mechanisms of action including denaturation of proteins within and between keratinocytes, and/or modification or disruption of lipids that results in increased lipid bilayer fluidity.

In a composition provided herein, the therapeutic agents and permeation enhancers are combined with matrix forming agents, to form compositions which form a hydrogel under suitable conditions. Such conditions may include exposure to body heat during administration (e.g., in the ear canal), or following mixing of two components of the composition or matrix-forming agent. The matrix forming agent is a compound or mixture of compounds that forms a gel after administration. The compositions are generally liquid at ambient conditions, however, once administered to a subject, the matrix forming agent or combination of matrix forming agents causes a phase transition to a hydrogel. Hydrogels have a highly porous structure that allows for the loading of drugs and other small molecules, and subsequent drug elution out of the gel creates a high local concentration in the surrounded tissues over an extended period. In certain embodiments, the drugs are loaded in the liquid composition. Hydrogels can conform and adhere to the shape of the surface to which they are applied and tend to be biocompatible.

For the compositions provided herein, the combination of the permeation enhancer with the matrix forming agent and therapeutic agent provides a composition with improved flux of the therapeutic agent, and also improved, or not significantly impaired, properties of the resulting hydrogel relative to the hydrogel formed by the composition in the absence of the permeation enhancer. For the compositions provided herein, the combination of the permeation enhancer with the matrix forming agent and therapeutic agent provides a composition with improved flux of the therapeutic agent, and additional improved properties including, but not limited to extended drug release, adherence of the composition to the tympanic membrane over time, degradation, or combinations thereof, and also improved, or not significantly impaired, properties of the resulting hydrogel relative to the hydrogel formed by the composition in the absence of the permeation enhancer.

In addition, with regard to the treatment of pain associated with AOM, it is hypothesized that the lack of efficient analgesic effects from ototopical drops was due to inability to penetrate the TM. The outermost layer in the TM, the stratum corneum, is impermeable to virtually all molecules except the small and moderately hydrophobic ones. The stratum corneum barrier can be disrupted by chemically and biologically active molecules and/or physical means¹². Chemical permeation enhancers (CPEs), in particular, have emerged as an effective means of enhancing small molecule flux across the TM^{13,14}. CPEs can reversibly increase the fluidity of the lipid bilayers in the interstitial space between impermeable corneocytes within the stratum corneum, greatly improving the transdermal delivery of molecules that would otherwise permeate poorly^{13,14}. Thus, a formulation combining CPEs and known anesthetics could enhance drug flux into and across an intact TM, and achieve effective analgesia for AOM.

Prior systems involve a transtympanic drug delivery system that utilizes a hydrogel compound, penta-block copolymer poloxamer 407-polybutylphospoester (P407-PBP) with three CPEs^{13,14}; sodium dodecyl sulfate (SDS), limonene (LIM), and bupivacaine-hydrochloride (BUP). That combination of CPEs brought ciprofloxacin across an intact TM and treated AOM in a chinchilla animal model successfully¹⁴. The formulation was administered as a single dose via the ear canal directly on the chinchillas' TM.

In the present composition, P407-PBP is used because of its robust reverse thermal gelation behavior¹⁴. The hydrogel-based formulation is an easy-to-apply liquid at room temperature, and gels quickly and firmly upon contacting the warm TM, holding the antibiotic and CPEs in place (i.e. on the TM). The sustained release and diffusion of drugs into the middle ear can thus be achieved by a single application of the formulation, resulting in high concentration of drug in the middle ear fluid¹⁴. Compositions and formulations for treatment of diseases and/or conditions (e.g., AOM and/or pain associated with AOM) disclosed herein also include therapeutic anesthetic agents bupivacaine also used as a CPE and the sodium channel blocker anesthetic agent of tetrodotoxin.

The optimal clinical applicability of such formulations is dependent on, but not limited to, a number of parameters. As a non-limiting example, such parameters include, but are not limited to, the concentration of particular permeation enhancers, flux of therapeutic agents, viscosity of the formulations for therapeutic application, rheological properties affecting gelation or affecting persistence on a barrier (e.g., the tympanic membrane), or adverse physiological reactions (e.g., adverse tissue reactions rendering the formulations unsafe or unsuitable for clinical application). Disclosed herein are formulations for clinical application (e.g., clinically applicable and including adequate flux of therapeutic agents).

In one aspect, provided herein are compositions comprising:
(a) a therapeutic agent or a combination of therapeutic agents;
(b) a permeation enhancer or a combination of permeation enhancers, wherein the permeation enhancer or combination of permeation enhancers increases the flux of the therapeutic agent or combination of therapeutic agents across a barrier; and
(c) a matrix forming agent or a combination of matrix forming agents, wherein the matrix forming agent or combination of matrix forming agents comprises a polymer;
   wherein:

the composition forms a gel at temperatures above a phase transition temperature; and

the phase transition temperature is less than about 37° C.;
wherein the composition comprises between about 0.5-20.0% wt/vol of a permeation enhancer that is sodium dodecyl sulfate;
wherein the composition comprises between about 0.5-7.5% wt/vol of a permeation enhancer that is bupivacaine that is one of the therapeutic agents;
wherein the composition comprises between about 0.5-12.0% wt/vol of a permeation enhancer that is limonene; and
wherein the composition comprises between about 9.0-20.0% wt/vol of a polymer that is poloxamer 407-poly(butoxy)phosphoester; and
   wherein the composition optionally further comprises between about 0.01-0.50% wt/vol of another therapeutic agent that is a local anesthetic.

In certain embodiments, provided herein are compositions comprising:
(a) a therapeutic agent or a combination of therapeutic agents;
(b) a permeation enhancer or a combination of permeation enhancers, wherein the permeation enhancer or combination of permeation enhancers increases the flux of the therapeutic agent or combination of therapeutic agents across a barrier; and
(c) a matrix forming agent or a combination of matrix forming agents, wherein the matrix forming agent or combination of matrix forming agents comprises a polymer;
   wherein:

the composition forms a gel at temperatures above a phase transition temperature; and

the phase transition temperature is less than about 37° C.;
wherein the composition comprises between about 0.5-5.5% wt/vol of a permeation enhancer that is sodium dodecyl sulfate;
wherein the composition comprises between about 0.5-7.5% wt/vol of a permeation enhancer that is bupivacaine that is one of the therapeutic agents;
wherein the composition comprises between about 0.5-10.0% wt/vol of a permeation enhancer that is limonene; and
wherein the composition comprises between about 9.0-19.0% wt/vol of a polymer that is poloxamer 407-poly(butoxy)phosphoester; and
wherein the composition comprises between about 0.01-0.50% wt/vol of the local anesthetic agent that is a sodium channel blocker.

In another aspect, provided herein are compositions comprising:
(a) a therapeutic agent or a combination of therapeutic agents;
(b) a permeation enhancer or a combination of permeation enhancers, wherein the permeation enhancer or combination of permeation enhancers increases the flux of the therapeutic agent or combination of therapeutic agents across a barrier; and
(c) a matrix forming agent or a combination of matrix forming agents, wherein the matrix forming agent or combination of matrix forming agents comprises a polymer;
   wherein:

the composition forms a gel at temperatures above a phase transition temperature; and

the phase transition temperature is less than about 37° C.;

wherein the composition comprises between about 0.5-5.5% wt/vol of a permeation enhancer that is sodium dodecyl sulfate;

wherein the composition comprises between about 0.5-1.5% wt/vol of a permeation enhancer that is bupivacaine;

wherein the composition comprises between about 2.0-12.0% wt/vol of a permeation enhancer that is limonene; and

wherein the composition comprises between about 9.0-19.0% wt/vol of a polymer that is poloxamer 407-poly(butoxy)phosphoester.

In certain embodiments, at least one of conditions (i), (ii), and (iii) are met:

(i) the composition can be extruded from a soft catheter ranging in size from a 10 gauge to 24 gauge, and from 1 inch to 5.25 inches, and the composition remains liquid;
(ii) the phase transition temperature of the composition is above about 15° C. and below about 37° C.; and
(iii) at 37° C., the storage modulus of the composition is greater than about 300 Pa, and the storage modulus is greater than the loss modulus of the composition.

In certain embodiments, at least one of conditions (i), (ii), and (iii) are met. In certain embodiments, the composition comprises between about 0.5-5.5% wt/vol of a permeation enhancer that is sodium dodecyl sulfate; the composition comprises between about 0.5-1.5% wt/vol of a permeation enhancer that is bupivacaine; and the composition comprises between about 2.0-12.0% wt/vol of a permeation enhancer that is limonene; and the composition comprises between about 9.0-19.0% wt/vol of a polymer that is poloxamer 407-poly(butoxy)phosphoester.

In certain embodiments, the composition comprises two therapeutic agents, including between about 0.01-0.50% wt/vol of another therapeutic agent that is a local anesthetic. In certain embodiments, the composition comprises between about 0.01-0.50% wt/vol of another therapeutic agent that is a local anesthetic that is a sodium channel blocker. In certain embodiments, the composition comprises a sodium channel blocker anesthetic agent (e.g., tetrodotoxin). In certain embodiments, the sodium channel blocker is a site 1 sodium channel blocker. In certain embodiments, the site 1 sodium channel blocker is tetrodotoxin.

In certain embodiments, the composition comprises between about between about 0.5-5.0% wt/vol of a permeation enhancer that is sodium dodecyl sulfate; the composition comprises between about 0.5-7.5% wt/vol of a permeation enhancer that is bupivacaine; and the composition comprises between about 0.5-3.5% wt/vol of a permeation enhancer that is limonene; the composition comprises between about 9.0-15.0% wt/vol of a polymer that is poloxamer 407-poly(butoxy)phosphoester; and the composition optionally comprises between about 0.01-0.50% wt/vol of another therapeutic agent that is a sodium channel blocker anesthetic agent of tetrodotoxin. In certain embodiments, the composition: about 1.0% wt/vol of sodium dodecyl sulfate; about 2.0% wt/vol of bupivacaine; about 2.0% wt/vol of limonene; about 12.0% wt/vol of poloxamer 407-poly(butoxy)phosphoester; and about 0.3% wt/vol of another therapeutic agent that is a sodium channel blocker anesthetic agent of tetrodotoxin.

In another aspect, provided herein are methods for treating a disease (e.g., an infectious disease, ear disease, bacterial infection) and/or a condition associated with the disease (e.g., pain associated with an infectious disease, ear disease, bacterial infection) comprising administering a composition comprising a therapeutic agent or a combination of therapeutic agents (e.g., antimicrobial agent, antibiotic, or anesthetic agent), permeation enhancers, and a matrix forming agent, as described herein, to a subject in need thereof.

In another aspect, provided herein are methods for treating an ear disease comprising administering a composition comprising a therapeutic agent or a combination of therapeutic agents (e.g., antimicrobial agent, antibiotic, or anesthetic agent), permeation enhancers, and a matrix forming agent, as described herein, to a subject in need thereof. In certain embodiments, the composition is administered into the ear canal or to the tympanic membrane. In certain embodiments, the disease is otitis media. In certain embodiments, the disease is an ear infection. In certain embodiments, the disease is a bacterial infection (e.g., a H. influenzae, S. pneumoniae, or M. catarhallis infection). In certain embodiments, the condition is pain. In certain embodiments, the condition is pain associated with the disease otitis media. In certain embodiments, the condition is pain associated with an ear infection. In certain embodiments, the condition is pain associated with a bacterial infection (e.g., a H. influenzae, S. pneumoniae, or M. catarhallis infection).

In another aspect, provided herein are methods for eradicating a biofilm comprising administering to a subject in need thereof, or contacting a biofilm with, a composition described herein.

In another aspect, provided herein are methods for inhibiting the formation of a biofilm comprising administering to a subject in need thereof, or contacting a surface with, a composition described herein.

In another aspect, provided herein are uses of compositions described herein to treat and/or prevent a disease or condition (e.g., an infectious disease, ear disease, bacterial infection) and/or a condition associated with the disease (e.g., pain; pain associated with an infectious disease, ear disease, bacterial infection) in a subject in need thereof, the use comprising administering to the subject a therapeutically effective amount of compositions described herein.

In another aspect, provided herein are pharmaceutical compositions comprising a composition described herein, and optionally a pharmaceutically acceptable excipient. In certain embodiments, the pharmaceutical compositions comprise a therapeutically effective amount of the composition for use in treating a disease in a subject in need thereof. In an additional aspect, provided herein are methods for delivering a composition described herein, the method comprising administering into an ear canal of a subject the composition, wherein the composition contacts the surface of a tympanic membrane. The composition may be administered with an eye dropper, syringe, double barrel syringe, or catheter (e.g., angiocatheter).

In an additional aspect, provided herein are kits comprising a container, a composition described herein, and instructions for administering the composition to a subject in need thereof. The kit may further comprise a device for administration of the composition to a subject, such as a dropper, syringe, catheter, double barrel syringe, or combination thereof.

The compositions, composition components (e.g., matrix forming agents, therapeutic agents, and permeation enhancers), methods, kits, and uses of the present disclosure may also incorporate any feature described in: Khoo et al., Biomaterials. (2013) 34, 1281-8; U.S. Pat. No. 8,822,410; U.S. patent application Ser. No. 12/993,358, filed May 19, 2009; U.S. patent application Ser. No. 11/734,537; filed Apr. 12, 2007; WIPO Patent Application No. PCT/US2009/003084, filed May 19, 2009, and WIPO Patent Application No. PCT/US2007/009121, filed Apr. 12, 2007, each of which is incorporated herein by reference.

The details of certain embodiments of the disclosure are set forth in the Detailed Description of Certain Embodiments, as described below. Other features, objects, and advantages of the disclosure will be apparent from the Definitions, Examples, Figures, and Claims.

### DEFINITIONS

### Chemistry Definitions

Definitions of specific functional groups and chemical terms are described in more detail below. The chemical elements are identified in accordance with the Periodic Table of the Elements, CAS version, Handbook of Chemistry and Physics, 75^{th} Ed., inside cover, and specific functional groups are generally defined as described therein. Additionally, general principles of organic chemistry, as well as specific functional moieties and reactivity, are described in Organic Chemistry, Thomas Sorrell, University Science Books, Sausalito, 1999; Smith and March March's Advanced Organic Chemistry, 5^{th} Edition, John Wiley & Sons, Inc., New York, 2001; Larock, Comprehensive Organic Transformations, VCH Publishers, Inc., New York, 1989; and Carruthers, Some Modern Methods of Organic Synthesis, 3^{rd} Edition, Cambridge University Press, Cambridge, 1987.

Compounds described herein can comprise one or more asymmetric centers, and thus can exist in various stereoisomeric forms, e.g., enantiomers and/or diastereomers. For example, the compounds described herein can be in the form of an individual enantiomer, diastereomer or geometric isomer, or can be in the form of a mixture of stereoisomers, including racemic mixtures and mixtures enriched in one or more stereoisomer. Isomers can be isolated from mixtures by methods known to those skilled in the art, including chiral high pressure liquid chromatography (HPLC) and the formation and crystallization of chiral salts; or preferred isomers can be prepared by asymmetric syntheses. See, for example, Jacques et al., Enantiomers, Racemates and Resolutions (Wiley Interscience, New York, 1981); Wilen et al., Tetrahedron 33:2725 (1977); Eliel, E. L. Stereochemistry of Carbon Compounds (McGraw-Hill, N Y, 1962); and Wilen, S. H. Tables of Resolving Agents and Optical Resolutions p. 268 (E. L. Eliel, Ed., Univ. of Notre Dame Press, Notre Dame, Ind. 1972). The disclosure additionally encompasses compounds as individual isomers substantially free of other isomers, and alternatively, as mixtures of various isomers.

Unless otherwise stated, structures depicted herein are also meant to include compounds that differ only in the presence of one or more isotopically enriched atoms. For example, compounds having the present structures except for the replacement of hydrogen by deuterium or tritium, replacement of ¹⁹F with ¹⁸F, or the replacement of ¹²C with ¹³C or ¹⁴C are within the scope of the disclosure. Such compounds are useful, for example, as analytical tools or probes in biological assays.

When a range of values is listed, it is intended to encompass each value and sub-range within the range. For example “C₁₋₆ alkyl” is intended to encompass, C₁, C₂, C₃, C₄, C₅, C₆, C₁₋₆, C₁₋₅, C₁₋₄, C₁₋₃, C₁₋₂, C₂₋₆, C₂₋₅, C₂₋₄, C₂₋₃, C₃₋₆, C₃₋₅, C₃₋₄, C₄₋₆, C₄₋₅, and C₅₋₆ alkyl.

The term “aliphatic” refers to alkyl, alkenyl, alkynyl, and carbocyclic groups. Likewise, the term “heteroaliphatic” refers to heteroalkyl, heteroalkenyl, heteroalkynyl, and heterocyclic groups.

The term “alkyl” refers to a radical of a straight-chain or branched saturated hydrocarbon group having from 1 to 10 carbon atoms (“C₁₋₁₀ alkyl”). In some embodiments, an alkyl group has 1 to 9 carbon atoms (“C₁₋₉ alkyl”). In some embodiments, an alkyl group has 1 to 8 carbon atoms (“C₁₋₈ alkyl”). In some embodiments, an alkyl group has 1 to 7 carbon atoms (“C₁₋₇ alkyl”). In some embodiments, an alkyl group has 1 to 6 carbon atoms (“C₁₋₆ alkyl”). In some embodiments, an alkyl group has 1 to 5 carbon atoms (“C₁₋₅ alkyl”). In some embodiments, an alkyl group has 1 to 4 carbon atoms (“C₁₋₄ alkyl”). In some embodiments, an alkyl group has 1 to 3 carbon atoms (“C₁₋₃ alkyl”). In some embodiments, an alkyl group has 1 to 2 carbon atoms (“C₁₋₂ alkyl”). In some embodiments, an alkyl group has 1 carbon atom (“C₁ alkyl”). In some embodiments, an alkyl group has 2 to 6 carbon atoms (“C₂₋₆ alkyl”). Examples of C₁-6 alkyl groups include methyl (C₁), ethyl (C₂), propyl (C₃) (e.g., n-propyl, isopropyl), butyl (C₄) (e.g., n-butyl, tert-butyl, sec-butyl, iso-butyl), pentyl (C₅) (e.g., n-pentyl, 3-pentanyl, amyl, neopentyl, 3-methyl-2-butanyl, tertiary amyl), and hexyl (C₆) (e.g., n-hexyl). Additional examples of alkyl groups include n-heptyl (C₇), n-octyl (C₈), and the like. Unless otherwise specified, each instance of an alkyl group is independently unsubstituted (an “unsubstituted alkyl”) or substituted (a “substituted alkyl”) with one or more substituents (e.g., halogen, such as F). In certain embodiments, the alkyl group is an unsubstituted C₁₋₁₀ alkyl (such as unsubstituted C₁₋₆ alkyl, e.g., —CH₃ (Me), unsubstituted ethyl (Et), unsubstituted propyl (Pr, e.g., unsubstituted n-propyl (n-Pr), unsubstituted isopropyl (i-Pr)), unsubstituted butyl (Bu, e.g., unsubstituted n-butyl (n-Bu), unsubstituted tert-butyl (tert-Bu or t-Bu), unsubstituted sec-butyl (sec-Bu), unsubstituted isobutyl (i-Bu)). In certain embodiments, the alkyl group is a substituted C₁₋₁₀ alkyl (such as substituted C₁₋₆ alkyl, e.g., —CF₃, Bn).

A “counterion” or “anionic counterion” is a negatively charged group associated with a positively charged group in order to maintain electronic neutrality. An anionic counterion may be monovalent (i.e., including one formal negative charge). An anionic counterion may also be multivalent (i.e., including more than one formal negative charge), such as divalent or trivalent. Exemplary counterions include halide ions (e.g., F⁻, Cl⁻, Br⁻, I⁻), NO₃⁻, ClO₄⁻, OH⁻, H₂PO₄⁻, HCO₃⁻, HSO₄⁻, sulfonate ions (e.g., methansulfonate, trifluoromethanesulfonate, p-toluenesulfonate, benzenesulfonate, 10-camphor sulfonate, naphthalene-2-sulfonate, naphthalene-1-sulfonic acid-5-sulfonate, ethan-1-sulfonic acid-2-sulfonate, and the like), carboxylate ions (e.g., acetate, propanoate, benzoate, glycerate, lactate, tartrate, glycolate, gluconate, and the like), BF₄⁻, PF₄⁻, PF₆⁻, AsF₆⁻, SbF₆⁻, B[3,5-(CF₃)₂C₆H₃]₄]⁻, B(C₆F₅)₄⁻, BPh₄⁻, Al(OC(CF₃)₃)₄⁻, and carborane anions (e.g., CB₁₁H₁₂⁻ or (HCB₁₁Me₅Br₆)⁻). Exemplary counterions which may be multivalent include CO₃²⁻, HPO₄²⁻, PO₄³⁻, B₄O₇²⁻, SO₄²⁻, S₂O₃²⁻, carboxylate anions (e.g., tartrate, citrate, fumarate, maleate, malate, malonate, gluconate, succinate, glutarate, adipate, pimelate, suberate, azelate, sebacate, salicylate, phthalates, aspartate, glutamate, and the like), and carboranes.

As used herein, use of the phrase “at least one instance” refers to 1, 2, 3, 4, or more instances, but also encompasses a range, e.g., for example, from 1 to 4, from 1 to 3, from 1 to 2, from 2 to 4, from 2 to 3, or from 3 to 4 instances, inclusive.

A “non-hydrogen group” refers to any group that is defined for a particular variable that is not hydrogen.

The term “polysaccharide” refers to a polymer composed of long chains of carbohydrate or monosaccharide units, or derivatives thereof (e.g., monosaccharides modified to comprise cross-linkable functional groups). Exemplary polysaccharides include, but are not limited to, glycans, glucans, starches, glycogens, arabinoxylans, celluloses, hemicelluloses, chitins, pectins, dextrans, pullulans, chrysolaminarins, curdlans, laminarins, lentinans, lichenins, pleurans, zymosans, glycosaminoglycans, dextrans, hyaluronic acids, chitosans, and chondroitins. The monosaccharide monomers of polysaccharides are typically connected by glysolidic linkages. Polysaccharides may be hydrolyzed to form oligosaccharides, disaccharides, and/or mono saccharides. The term “carbohydrate” or “saccharide” refers to an aldehydic or ketonic derivative of polyhydric alcohols. Monosaccharides are the simplest carbohydrates in that they cannot be hydrolyzed to smaller carbohydrates. Most monosaccharides can be represented by the general formula C_{y}H_{2y}O_{y} (e.g., C₆H₁₂O₆ (a hexose such as glucose)), wherein y is an integer equal to or greater than 3. Certain polyhydric alcohols not represented by the general formula described above may also be considered monosaccharides. For example, deoxyribose is of the formula C₅H₁₀O₄ and is a monosaccharide. Monosaccharides usually consist of five or six carbon atoms and are referred to as pentoses and hexoses, receptively. If the monosaccharide contains an aldehyde it is referred to as an aldose; and if it contains a ketone, it is referred to as a ketose. Monosaccharides may also consist of three, four, or seven carbon atoms in an aldose or ketose form and are referred to as trioses, tetroses, and heptoses, respectively. Glyceraldehyde and dihydroxyacetone are considered to be aldotriose and ketotriose sugars, respectively. Examples of aldotetrose sugars include erythrose and threose; and ketotetrose sugars include erythrulose. Aldopentose sugars include ribose, arabinose, xylose, and lyxose; and ketopentose sugars include ribulose, arabulose, xylulose, and lyxulose. Examples of aldohexose sugars include glucose (for example, dextrose), mannose, galactose, allose, altrose, talose, gulose, and idose; and ketohexose sugars include fructose, psicose, sorbose, and tagatose. Ketoheptose sugars include sedoheptulose. Each carbon atom of a monosaccharide bearing a hydroxyl group (—OH), with the exception of the first and last carbons, is asymmetric, making the carbon atom a stereocenter with two possible configurations (R or S). Because of this asymmetry, a number of isomers may exist for any given monosaccharide formula. The aldohexose D-glucose, for example, has the formula C₆H₁₂O₆, of which all but two of its six carbons atoms are stereogenic, making D-glucose one of the 16 (i.e., 2⁴) possible stereoisomers. The assignment of D or L is made according to the orientation of the asymmetric carbon furthest from the carbonyl group: in a standard Fischer projection if the hydroxyl group is on the right the molecule is a D sugar, otherwise it is an L sugar. The aldehyde or ketone group of a straight-chain monosaccharide will react reversibly with a hydroxyl group on a different carbon atom to form a hemiacetal or hemiketal, forming a heterocyclic ring with an oxygen bridge between two carbon atoms. Rings with five and six atoms are called furanose and pyranose forms, respectively, and exist in equilibrium with the straight-chain form. During the conversion from the straight-chain form to the cyclic form, the carbon atom containing the carbonyl oxygen, called the anomeric carbon, becomes a stereogenic center with two possible configurations: the oxygen atom may take a position either above or below the plane of the ring. The resulting possible pair of stereoisomers is called anomers. In an a anomer, the —OH substituent on the anomeric carbon rests on the opposite side (trans) of the ring from the —CH₂OH side branch. The alternative form, in which the —CH₂OH substituent and the anomeric hydroxyl are on the same side (cis) of the plane of the ring, is called a β anomer. The term carbohydrate also includes other natural or synthetic stereoisomers of the carbohydrates described herein.

These and other exemplary substituents are described in more detail in the Detailed Description, Examples, and Claims. The disclosure is not intended to be limited in any manner by the above exemplary listing of substituents.

### Other Definitions

Animal: The term animal, as used herein, refers to humans as well as non-human animals, including, for example, mammals, birds, reptiles, amphibians, and fish. Preferably, the non-human animal is a mammal (e.g., a rodent, a mouse, a rat, a rabbit, a monkey, a dog, a cat, a primate, or a pig). A non-human animal may be a transgenic animal.

Approximately or About: As used herein, the terms “approximately” or “about” in reference to a number are generally taken to include numbers that fall within a range of 5%, 10%, 15%, or 20% in either direction (greater than or less than) of the number unless otherwise stated or otherwise evident from the context (except where such number would be less than 0% or exceed 100% of a possible value).

Biocompatible: As used herein, the term “biocompatible” refers to substances that are not toxic to cells. In some embodiments, a substance is considered to be “biocompatible” if its addition to cells in vivo does not induce inflammation and/or other adverse effects in vivo. In some embodiments, a substance is considered to be “biocompatible” if its addition to cells in vitro or in vivo results in less than or equal to about 50%, about 45%, about 40%, about 35%, about 30%, about 25%, about 20%, about 15%, about 10%, about 5%, or less than about 5% cell death.

Biodegradable: As used herein, the term “biodegradable” refers to substances that are degraded under physiological conditions. In some embodiments, a biodegradable substance is a substance that is broken down by cellular machinery. In some embodiments, a biodegradable substance is a substance that is broken down by chemical processes.

Optically transparent: As used herein, the term “optically transparent” refers to substances through which light passes through with little or no light being absorbed or reflected. In some embodiments, optically transparent refers to substances through which light passes through with no light being absorbed or reflected. In some embodiments, optically transparent refers to substances through which light passes through with little light being absorbed or reflected. In some embodiments, an optically transparent substance is substantially clear. In some embodiments, an optically transparent substance is clear.

Effective amount: In general, the “effective amount” of an active agent refers to an amount sufficient to elicit the desired biological response. As will be appreciated by those of ordinary skill in this art, the effective amount of a compound of the disclosure may vary depending on such factors as the desired biological endpoint, the pharmacokinetics of the compound, the disease being treated, the mode of administration, and the patient. The effective amount of a compound used to treat infection is the amount needed to kill or prevent the growth of the organism(s) responsible for the infection.

In vitro: As used herein, the term “in vitro” refers to events that occur in an artificial environment, e.g., in a test tube or reaction vessel, in cell culture, etc., rather than within an organism (e.g. animal, plant, and/or microbe).

In vivo: As used herein, the term “in vivo” refers to events that occur within an organism (e.g. animal, plant, and/or microbe).

Suffering from: An individual who is “suffering from” a disease, disorder, and/or condition has been diagnosed with or displays one or more symptoms of the disease, disorder, and/or condition.

Treating: As used herein, the term “treating” refers to partially or completely alleviating, ameliorating, relieving, delaying onset of, inhibiting progression of, reducing severity of, and/or reducing incidence of one or more symptoms or features of a particular disease, disorder, and/or condition. For example, “treating” a microbial infection may refer to inhibiting survival, growth, and/or spread of the microbe. Treatment may be administered to a subject who does not exhibit signs of a disease, disorder, and/or condition and/or to a subject who exhibits only early signs of a disease, disorder, and/or condition for the purpose of decreasing the risk of developing pathology associated with the disease, disorder, and/or condition. In some embodiments, treatment comprises delivery of an inventive vaccine nanocarrier to a subject.

Therapeutic agent: Also referred to as a “drug” is used herein to refer to an agent that is administered to a subject to treat a disease, disorder, or other clinically recognized condition that is harmful to the subject, or for prophylactic purposes, and has a clinically significant effect on the body to treat or prevent the disease, disorder, or condition. Therapeutic agents include, without limitation, agents listed in the United States Pharmacopeia (USP), Goodman and Gilman's The Pharmacological Basis of Therapeutics, 10^{th} Ed., McGraw Hill, 2001; Katzung, B. (ed.) Basic and Clinical Pharmacology, McGraw-Hill/Appleton & Lange; 8th edition (Sep. 21, 2000); Physician's Desk Reference (Thomson Publishing), and/or The Merck Manual of Diagnosis and Therapy, 17^{th} ed. (1999), or the 18th Ed. (2006) following its publication, Mark H. Beers and Robert Berkow (Eds.), Merck Publishing Group, or, in the case of animals, The Merck Veterinary Manual, 9^{th} ed., Kahn, C. A. (Ed.), Merck Publishing Group, 2005.

Diagnostic agent: As used herein, the term “diagnostic agent” refers to an agent that is administered to a subject to aid in the diagnosis of a disease, disorder, or condition. In some embodiments, a diagnostic agent is used to define and/or characterize the localization of a pathological process. Diagnostic agents include X-ray contrast agents, radioactive isotopes, and dyes.

Surfactant: As used herein, the term “surfactant” refers to any agent which preferentially absorbs to an interface between two immiscible phases, such as the interface between water and an organic solvent, a water/air interface, or an organic solvent/air interface. Surfactants usually possess a hydrophilic moiety and a hydrophobic moiety. Surfactants may also promote flux of a therapeutic or diagnostic agent across a biological membrane, e.g., a tympanic membrane.

Terpenes: As used herein, the term “terpene” refers to any agent derived, e.g., biosynthetically, or thought to be derived from unit(s) of isoprene (a five carbon unit). For example, isoprene units of terpenes may be linked together to form linear chains or they may be arranged to form rings. Typically, the terpenes disclosed herein promote flux of a therapeutic or diagnostic agent across a biological membrane, e.g., a tympanic membrane. Terpenes may be naturally derived or synthetically prepared.

The terms “composition” and “formulation” are used interchangeably.

### BRIEF DESCRIPTION OF THE DRAWINGS

The accompanying drawings are not intended to be drawn to scale. In the drawings, each identical or nearly identical component that is illustrated in various figures is represented by a like numeral. For purposes of clarity, not every component may be labeled in every drawing. The patent or application file contains at least one drawing executed in color. Copies of this patent or patent application publication with color drawing(s) will be provided by the Office upon request and payment of the necessary fee. In the drawings:

FIG. 1 shows exemplary optimization of exemplary compositions described herein, comprising a therapeutic agent, permeation enhancers, and matrix forming agent (e.g. for synergy in increasing the peak effect, i.e. the maximum drug flux across a barrier like the tympanic membrane).

FIG. 2 shows a summary of rheology data for exemplary viable compositions. Provided for each of the compositions are the temperature for gelation (° C.), the average storage modulus (G′), and standard deviation of the storage modulus.

FIG. 3 shows rheology data for a composition with 12% Poloxamer 407-poly(butoxy)phosphoester (“PBP”), 1% sodium dodecyl sulfate (“SDS”), 1% bupivacaine (“BUP”), and 10% limonene (“LIM”). Provided are the average storage modulus (“storage”) and average loss modulus (“loss”) plotted against the temperature of the composition. Error bars represent standard deviations.

FIG. 4 shows rheology for a composition with 12% PBP-5% SDS-1% BUP-4% LIM. Provided are the average storage modulus (“store ave.”) and average loss modulus (“loss ave.”) plotted against the temperature of the composition. Error bars represent standard deviations.

FIG. 5 shows rheology data for a composition with 15% PBP-5% SDS-1% BUP-4% LIM. Provided are the average storage modulus (“store ave.”) and average loss modulus (“loss ave.”) plotted against the temperature of the composition. Error bars represent standard deviations.

FIG. 6 shows rheology data for a composition with 10% PBP-5% SDS-0.5% BUP-4% LIM. Provided are the average storage modulus (“store ave.”) and average loss modulus (“loss ave.”) plotted against the temperature of the composition. Error bars represent standard deviations.

FIGS. 7A and 7B show cumulative permeation of bupivacaine hydrochloride (BUP) across the tympanic membrane from formulations containing 2CPE-[P407-PBP]. (FIG. 7A) Time course of cumulative permeation of BUP achieved by BUP-2CPE-[P407-PBP] with different BUP concentrations over 48 hours. BUP was not soluble in 2CPE-[P407-PBP]beyond 4%. Therefore the formulations, 7.5% BUPₛᵤₛₚ-2CPE-[P407-PBP] and 15% BUPₛᵤₛₚ-2CPE-[P407-PBP] were suspensions, which is indicated by † in the plot. Arrows indicate data graphed in FIG. 7B. (FIG. 7B) Effect of bupivacaine concentration of cumulative permeation across the TM at 6 and 48 hours, derived from data denoted by arrows in FIG. 7A. Data are medians±interquartile ranges (n=4).

FIGS. 8A and 8B show cumulative permeation of tetrodotoxin (TTX) across the tympanic membrane from formulations containing 2CPE-[P407-PBP]. FIG. 8A shows trans-tympanic permeation of TTX from formulations containing 0.02, 0.03, 0.16, and 0.32% TTX, which corresponds to 0.5, 1, 5, and 10 mM TTX, over 48 hours. FIG. 8B shows the dependence of TTX permeation on the drug concentration of the hydrogel formulations. Data are medians±interquartile ranges (n=4).

FIGS. 9A and 9B show cumulative ex vivo permeation of (FIG. 9A) BUP and (FIG. 9B) TTX across the tympanic membrane from formulations containing both compounds and [P407-PBP]. Data are medians±interquartile ranges (n=4).

FIG. 10 shows cumulative permeation of bupivacaine free base and BUP across the tympanic membrane. BUP was not soluble in 2CPE-[P407-PBP] beyond 4%. Therefore 15% BUPₛᵤₛₚ-2CPE-[P407-PBP] was a suspension, which is indicated by † in the plot. Data are medians±interquartile ranges (n=4).

FIG. 11 shows representative hematoxylin and eosin (H&E)-stained sections of TMs treated under different conditions. Scale bar represents 12 μm.

FIG. 12 shows representative H&E-stained sections of the healthy external auditory meatus, of external auditory meatus processed 24 hours after exposing to 10%[bupivacaine free base]-LIM, and of external auditory meatus treated with 4% BUP-2CPE-[P407-PBP] or 15% BUP-2CPE-[P407-PBP] for 7 days. Scale bar represents 50 μm. Inset: enlarged image highlighting inflammatory cells; black arrow points to a neutrophil; white arrow with black outline points to a lymphocyte; scale bar within the inset represents 10 μm.

FIG. 13 shows cumulative in vitro release of Cip from the hydrogel formulations under infinite sink conditions. Eight milligrams of Cip were contained in each gel and solution at time zero. Data are means±SD (n=4).

FIGS. 14A and 14B show construction of an isobologram. (FIG. 14A) Concentration (Conc.)-response curves are used to identify isoboles, i.e. concentrations achieving the same effect (R). In this work, the principal R is V_{CIP48}. (FIG. 14B) Isobolographic analysis. See discussion below for explanation. Cₓ and C_{y} are the equivalent doses for drugs X and Y. The diagonal line is the line of additivity, also known as the isobole.

FIGS. 15A to 15F show performance of pairs of CPEs. (FIGS. 15A to 15C) Cumulative Cip permeation across the TM over 48 hours (V_{CIP48}) from CPPB containing varying concentrations of CPEs, singly (black curves), or in combination with other CPEs (gray points on the graphs). The gray points represent the same data in all panels. Data are means±SD (n=4). * 5% BUP and 30% SDS were suspensions, not homogeneous solutions. † p<0.05 for the comparisons between CPE combinations and the single CPE that is the subject of the panel; †† p<0.1. (FIGS. 15D to 15F) Isobolograms for combinations of (FIG. 15D) SDS and/or LIM that achieved V_{CIP48}=0.39 mg, (FIG. 15E) SDS and/or BUP that achieved V_{CIP48}=0.24 mg, and (FIG. 15F) BUP and/or LIM that achieved V_{CIP48}=0.22 mg. The data are derived from FIGS. 15A-15C.

FIGS. 16A-16C show cumulative Cip permeation across the TM over 6 hours (V_{CIP6}) from CPPB containing varying concentrations of CPEs, singly (black curves), or in combination with other CPEs (gray points on the graphs). The gray points represent the same data in all panels. Data are means±SD (n=4). * 5% BUP and 30% SDS were suspensions, not homogeneous solutions. † p<0.05 for the comparisons between CPE combinations and the single CPE that is the subject of the panel; †† p<0.1.

FIGS. 17A to 17C show concentration-response curves for ciprofloxacin permeation across the TM after 48 hours (i.e. V_{CIP48}) from CPPB containing (FIG. 17A) SDS, (FIG. 17B) LIM, and (FIG. 17C) BUP. Data were fitted to a three-parameter hyperbolic function model (black line) using Equation (1). The fitting parameters are listed in Table 1. Data points (gray dots) originate from FIG. 14. Note that the y-axis scale for FIG. 17C is different from those for FIG. 17A and FIG. 17B.

FIG. 18A shows an isobologram plot for combinations of SDS and/or LIM and/or BUP that achieved V_{CIP48}=0.4 mg. The surface is derived from the isobole for the three CPEs, from FIGS. 14A to 14C. The gray dot is the measured V_{CIP48} from a combination of all three CPEs. FIG. 18B shows the effect of CPE combinations on the peak V_{CIP48}. The peak flux for CPEs happened at 4%, 20%, and 1% for LIM, SDS, and BUP respectively; the combination column included 4% LIM, 1% BUP, and 20% SDS. Data are means±SD (n=4).

FIG. 19 shows molecular structures of sodium dodecyl sulfate (SDS), limonene (LIM), bupivacaine hydrochloride (BUP), and poloxamer 407-polybutylphosphoester (P407-PBP).

FIGS. 20A and 20B shows the synthesis of P407-PBP. FIG. 20A shows the NMR of pentablock copolymers. The chemical shifts (δ, in ppm) for the peaks corresponding to the hydrogens in italics in the following list of polymers is provided below. t/m/broad indicate the shape of a peak (i.e., triplet, multiple, broad). CDCl₃ was the solvent. ¹H NMR (CDCl₃, ppm): δ 0.90-0.96 (t, 3H, CH₂CH₂CH₂CH₃), 1.14 (m, 3H, CH₂CH(CH₃)O), 1.36-1.46 (m, 2H, CH₂CH₂CH₂CH₃), 1.62-1.72 (m, 2H, CH₂CH₂CH₂CH₃), 3.36-3.42 (m, CH₂CH(CH₃)O), 3.48-3.58 (m, 2H, CH₂CH(CH₃)O), 3.65 (m, 4H, OCH₂CH₂O), 4.04-4.14 (m, 2H, PCH₂CH₂CH₂CH₃), 4.16-4.30 (broad, 4H, POCH₂CH₂O). FIG. 20B shows the FTIR spectra of P407 and P407-PBP. FIG. 20B shows the FTIR of tri- and penta-block copolymers. The peaks are assigned as follows: 2650-3020 cm⁻¹: C—H stretch from CH₂ and CH₃ groups; 1466 cm⁻¹: C—H bend from CH₂ and CH₃ groups; 1328-1400 cm⁻¹: C—H stretch and bend from isopropyl groups; 1279 cm⁻¹: C—O and C—C stretch (crystalline phase); 1241 cm⁻¹: asymmetric C—O—C stretch; 1144 cm⁻¹: symmetric C—O—C stretch; 1103 cm⁻¹: C—O stretch; 1061 cm⁻¹: CO—C axial deform; 1030 cm⁻¹: P—O stretch; 964 cm⁻¹: ═C—H bend; 845 cm⁻¹: C—CH deform.

### DETAILED DESCRIPTION OF CERTAIN EMBODIMENTS OF THE DISCLOSURE

Provided herein are compositions and methods for administering a therapeutic agent to a subject through a barrier. In some embodiments, the composition is for administering a therapeutic agent to the ear of a subject, and the barrier is a tympanic membrane. The compositions and methods provide for the efficient delivery of the agent to the middle and/or inner ear of the subject. In one aspect, the composition comprises a combination of a permeation enhancer, a therapeutic agent or a combination of therapeutic agents, and a matrix forming agent. The permeation enhancer increases the flux of the therapeutic agent or a combination of therapeutic agents across the barrier (e.g., tympanic membrane), compared to the flux for a composition lacking the permeation enhancer. In various aspects, the composition is a single application composition for localized, sustained delivery of a therapeutic agent or a combination of therapeutic agents across the tympanic membrane. In various aspects, the composition is a multiple application composition for localized, sustained delivery of a therapeutic agent across the tympanic membrane. The compositions and methods described herein are particularly useful in treating otitis media and/or pain associated with otitis media by providing sustained release and delivery of an antibiotic to the middle ear.

In one aspect, provided herein are compositions comprising:
(a) a therapeutic agent or a combination of therapeutic agents;
(b) a permeation enhancer or a combination of permeation enhancers, wherein the permeation enhancer or combination of permeation enhancers increases the flux of the therapeutic agent or combination of therapeutic agents across a barrier; and
(c) a matrix forming agent or a combination of matrix forming agents, wherein the matrix forming agent or combination of matrix forming agents comprises a polymer;
   wherein:

the composition forms a gel at temperatures above a phase transition temperature; and

the phase transition temperature is less than about 37° C.;
wherein the composition comprises between about 0.5-20.0% wt/vol of a permeation enhancer that is sodium dodecyl sulfate;
wherein the composition comprises between about 0.5-7.5% wt/vol of a permeation enhancer that is bupivacaine that is one of the therapeutic agents;
wherein the composition comprises between about 0.5-12.0% wt/vol of a permeation enhancer that is limonene; and
wherein the composition comprises between about 9.0-20.0% wt/vol of a polymer that is poloxamer 407-poly(butoxy)phosphoester; and
wherein the composition optionally further comprises between about 0.01-0.50% wt/vol of another therapeutic agent that is a local anesthetic.

In certain embodiments, provided herein are compositions comprising:
(a) a therapeutic agent or a combination of therapeutic agents;
(b) a permeation enhancer or a combination of permeation enhancers, wherein the permeation enhancer or combination of permeation enhancers increases the flux of the therapeutic agent or combination of therapeutic agents across a barrier; and
(c) a matrix forming agent or a combination of matrix forming agents, wherein the matrix forming agent or combination of matrix forming agents comprises a polymer;
   wherein:

the composition forms a gel at temperatures above a phase transition temperature; and

the phase transition temperature is less than about 37° C.;
wherein the composition comprises between about 0.5-5.5% wt/vol of a permeation enhancer that is sodium dodecyl sulfate;
wherein the composition comprises between about 0.5-7.5% wt/vol of a permeation enhancer that is bupivacaine that is one of the therapeutic agents;
wherein the composition comprises between about 0.5-10.0% wt/vol of a permeation enhancer that is limonene; and
wherein the composition comprises between about 9.0-19.0% wt/vol of a polymer that is poloxamer 407-poly(butoxy)phosphoester; and
wherein the composition comprises between about 0.01-0.50% wt/vol of the local anesthetic agent that is a sodium channel blocker.

In one aspect, provided herein are compositions comprising: a therapeutic agent or a combination of therapeutic agents;
(b) a permeation enhancer or a combination of permeation enhancers, wherein the permeation enhancer or combination of permeation enhancers increases the flux of the therapeutic agent or combination of therapeutic agents across a barrier; and
(c) a matrix forming agent or a combination of matrix forming agents, wherein the matrix forming agent or combination of matrix forming agents comprises a polymer;
   wherein:

the composition forms a gel at temperatures above a phase transition temperature; and

the phase transition temperature is less than about 37° C.;

wherein the composition comprises between about 0.5-5.5% wt/vol of a permeation enhancer that is sodium dodecyl sulfate;

wherein the composition comprises between about 0.5-1.5% wt/vol of a permeation enhancer that is bupivacaine;

wherein the composition comprises between about 2.0-12.0% wt/vol of a permeation enhancer that is limonene; and

wherein the composition comprises between about 9.0-19.0% wt/vol of a polymer that is poloxamer 407-poly(butoxy)phosphoester.

In certain embodiments, at least one of conditions (i), (ii), and (iii) are met:

(i) the composition can be extruded from a soft catheter ranging in size from a 16 gauge to 24 gauge, and from 1 inch to 5.25 inch soft catheter, and the composition remains liquid;
(ii) the phase transition temperature of the composition is above about 15° C. and below about 37° C.; and
(iii) at 37° C., the storage modulus of the composition is greater than about 300 Pa, and the storage modulus is greater than the loss modulus of the composition.

In certain embodiments, condition (i), the composition can be extruded from a soft catheter ranging in size from a 10 gauge to a 24 gauge, and from 1 inch to 5.25 inch soft catheter, and the composition remains liquid, is met. In certain embodiments, condition (i), the composition can be extruded from a soft catheter ranging in size from a 16 gauge to a 24 gauge, and from 1.16 inch to 5.25 inch soft catheter, and the composition remains liquid, is met. In certain embodiments, condition (i), the composition can be extruded from a soft catheter ranging in size from a 16 gauge to 24 gauge, and from 1 inch to 5.25 inch soft catheter, and the composition remains liquid, is met. In certain embodiments, condition (i), the composition can be extruded from a soft catheter ranging in size from a 16 gauge to a 18 gauge, and from 1.16 inch to 1.88 inch soft catheter, and the composition remains liquid, is met. In certain embodiments, in condition (i), the soft catheter is an 18 gauge, 1.88 inch soft catheter, is met. In certain embodiments, in condition (i), the soft catheter is a 10 gauge, 1 inch soft catheter, is met. In certain embodiments, in condition (i), the soft catheter is a 16 gauge, 1.16 inch soft catheter, is met. In certain embodiments, in condition (i), the soft catheter is a 20 gauge, 3 inch soft catheter, is met. In certain embodiments, in condition (i), the soft catheter is a 22 gauge, 3.25 inch soft catheter, is met. In certain embodiments, in condition (i), the soft catheter is a 24 gauge, 5.25 inch soft catheter, is met.

In certain embodiments, condition (ii), the phase transition temperature of the composition is above about 15° C. and below about 37° C., is met. In certain embodiments, condition (ii), the phase transition temperature of the composition is above about 18° C. and below about 37° C., is met. In certain embodiments, condition (ii), the phase transition temperature of the composition is above about 20° C. and below about 37° C., is met.

In certain embodiments, condition (iii), at 37° C., the storage modulus of the composition is greater than about 300 Pa, and the storage modulus is greater than the loss modulus of the composition, is met. In certain embodiments, condition (iii), at 37° C., the storage modulus of the composition is greater than about 305 Pa, and the storage modulus is greater than the loss modulus of the composition, is met. In certain embodiments, condition (iii), at 37° C., the storage modulus of the composition is greater than about 310 Pa, and the storage modulus is greater than the loss modulus of the composition, is met. In certain embodiments, condition (iii), at 37° C., the storage modulus of the composition is greater than about 312 Pa, and the storage modulus is greater than the loss modulus of the composition, is met.

In certain embodiments, both conditions (i) and (ii) are met. In certain embodiments, both conditions (ii) and (iii) are met. In certain embodiments, both conditions (i) and (iii) are met. In certain embodiments, each of conditions (i), (ii), and (iii) are met.

In certain embodiments, the therapeutic agent is a single therapeutic agent. In certain embodiments, the therapeutic agent is combination of two or more therapeutic agents (e.g., two, three, four). In certain embodiments, the permeation enhancer is a single therapeutic agent. In certain embodiments, the therapeutic agent is combination of two or more therapeutic agents (e.g., two, three, four). In certain embodiments, the matrix forming agent is a single matrix forming agent. In certain embodiments, the matrix forming agent is a combination of two or more matrix forming agents (e.g., two, three, four). In certain embodiments, a therapeutic agent or permeation enhancer may act as both a therapeutic agent and a permeation enhancer. In certain embodiments, a therapeutic agent may act as both a therapeutic agent and a permeation enhancer. In certain embodiments, a permeation enhancer may act as both a therapeutic agent and a permeation enhancer. In certain embodiments, a local anesthetic may act as both a therapeutic agent and a permeation enhancer. In certain embodiments, an amino amide or amino ester local anesthetic may act as both a therapeutic agent and a permeation enhancer. In certain embodiments, an amino amide or amino ester local anesthetic may act as both a therapeutic agent and a permeation enhancer. In certain embodiments, an amino ester local anesthetic may act as both a therapeutic agent and a permeation enhancer. In certain embodiments, bupivacaine may act as both a therapeutic agent and a permeation enhancer. In certain embodiments, tetracaine may act as both a therapeutic agent and a permeation enhancer.

In certain embodiments, the permeation enhancer or combination of permeation enhancers is present in an amount effective to increase the flux of the therapeutic agent across a barrier compared to the reference composition (e.g., the composition without the permeation enhancer). In certain embodiments, the permeation enhancer or combination of permeation enhancers is present in an amount effective to increase the flux of the therapeutic agent across a barrier compared to the reference composition (e.g., the composition without the permeation enhancer) by at least about 1.05 fold, at least about 1.10 fold, at least about 1.2 fold, at least about, at least about 1.3 fold, at least about 1.4 fold, at least about 1.5 fold, at least about 1.6 fold, at least about 1.7 fold, at least about 1.8 fold, or at least about 1.9 fold. In certain embodiments, the permeation enhancer or combination of permeation enhancers is present in an amount effective to increase the flux of the therapeutic agent across a barrier compared to a reference composition by at least about 2 fold, at least about 2.5 fold, at least about 3 fold, at least about 4 fold, at least about 5 fold, at least about 10 fold, at least about 25 fold, at least about 50 fold, at least about 100 fold, at least about 250 fold, at least about 500 fold, or at least about 1000 fold. In certain embodiments, the permeation enhancer or combination of permeation enhancers is present in an amount effective to increase the flux of the therapeutic agent across a barrier compared to a reference composition by between about 1.5 fold and about 100 fold.

In certain embodiments, the matrix forming agent or a combination of matrix forming agents comprises a polymer that is poloxamer 407-poly(butoxy)phosphoester. In certain embodiments, the polymer is of the formula:

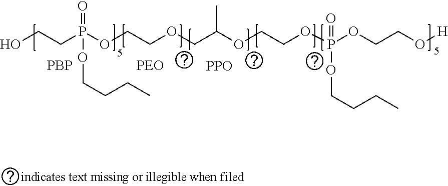

(poloxamer 407-poly(butoxy)phosphoester; also referred to as “PBP-P407” or “PBP”).

The composition may be a liquid prior to warming above the phase transition temperature. In some embodiments, the phase transition temperature is at or below the body temperature of a subject (e.g., about 37° C.). Thus, the composition may form a gel when administered to a subject, e.g., when the composition contacts a biological surface.

In some embodiments, the phase transition temperature is between about 15° C. and about 37° C., between about 20° C. and about 37° C., between about 25° C. between about 30° C. and about 37° C., between about 30° C. and about 35° C., or between about 35° C. and about 40° C. In some embodiments, the phase transition temperature is between about 20° C. and about 37° C. In some embodiments, the phase transition temperature is between about 0° C. and about 60° C., between about 10° C. and about 50° C., between about 20° C. and about 40° C., or between about 25° C. and about 35° C. In some embodiments, the phase transition temperature is between about 20° C. and 25° C., between about 25° C. and about 30° C., between about 30° C. and about 35° C., or between about 35° C. and about 40° C. In some embodiments, the phase transition temperature is between about 10° C. and about 50° C. In some embodiments, the phase transition temperature is between about 20° C. and about 40° C. In some embodiments, the phase transition temperature is between about 15° C. and about 40° C.

In certain embodiments, the composition is applied to a surface of temperature equal to or above the phase transition temperature. In some embodiments, the surface is a biological surface. In certain embodiments, the surface is skin. In certain embodiments, the surface is a surface in the ear canal of a subject. In certain embodiments, the surface is a tympanic membrane. In certain embodiments, the surface is a surface in the respiratory tract of a subject (e.g., in the nasal cavity or buccal cavity). In certain embodiments, the surface is a surface in the mouth (e.g., surface of teeth or gums) of a subject. The composition may be administered to an interior body surface, for example, by intradermal or interdermal delivery or during a surgical procedure. In certain embodiments, the surface is an intradermal surface. In certain embodiments, the surface is the surface of an organ (e.g., heart, lung, spleen, pancreas, kidney, liver, stomach, intestine, bladder). In certain embodiments, the surface is connective tissue. In certain embodiments, the surface is muscle tissue (e.g., smooth muscle, skeletal muscle, cardiac muscle). In certain embodiments, the surface is nervous tissue (e.g., brain, spinal cord). In certain embodiments, the surface is epithelial tissue. In certain embodiments, the surface is a surface of the alimentary canal (e.g., colon, rectum). In certain embodiments, the surface is epithelial tissue. In certain embodiments, the surface is a surface of the reproductive tract (e.g., vagina, cervix). In certain embodiments, the surface is bone. In certain embodiments, the surface is vascular tissue. In certain embodiments, the surface is a wound bed. In certain embodiments, the surface is a biofilm. In certain embodiments, the surface is hair or fur. In certain embodiments, the surface is the surface of a medical implant.

In certain embodiments, the composition is useful in treating a disease. In some embodiments, the composition is useful in treating an infectious disease. In some embodiments, the composition is useful in treating an ear disease (e.g., the barrier is the tympanic membrane). In some embodiments, the composition is useful in treating otitis media. In certain embodiments, the composition is useful in treating (e.g., sustained treating of) pain. In certain embodiments, the composition is useful in treating (e.g., sustained treating of) pain associated with a disease. In some embodiments, the composition is useful in treating (e.g., sustained treating of) pain associated with an infectious disease. In some embodiments, the composition is useful in treating (e.g., sustained treating of) pain associated with an ear disease (e.g., the barrier is the tympanic membrane). In some embodiments, the composition is useful in treating (e.g., sustained treating of) pain associated with otitis media.

As described, the gelation temperature (phase transition temperature) of the composition is one factor in determining whether the suitability of the composition (e.g., to allow for sustained delivery to the tympanic membrane). The temperature at which the storage modulus exceeds the loss modulus is considered the gelation temperature. Compositions herein may have a gelation temperature lower or higher than 37° C., but preferably lower than 37° C. to accelerate gelation right after administration upon exposure of the composition, in particular the matrix forming agent, to body heat.

The timing of the sol-gel transition will impact the ease of administration. In general a faster in situ transition is useful for administration to subjects (e.g., children resisting compliance). In certain embodiments, the composition gels within about 5 s, about 10 s, about 20 s, about 30 s, about 1 minute, about 5 minutes, or about 10 minutes of administration (e.g., to the ear canal). In some embodiments, the composition gels in the range of about 1 s to about 20 s after administration.

In certain embodiments, the composition is stored cold (e.g., refrigerated at about 5° C.) prior to administration. Cold storage may be useful for compositions with gelation temperatures below room temperature to prevent gelation prior to administration or during handling.

The compositions provided herein include a permeation enhancer (e.g., a surfactant, terpene), a therapeutic agent or a combination of therapeutic agents (e.g., an antibiotic, anesthetic agent), and a matrix forming agent (e.g., PBP-poloxamer 407). The permeation enhancer is an agent that alters the stratum corneum of the tympanic membrane to increase the flux of the therapeutic agent across the tympanic membrane. The permeation enhancer facilitates delivery of the therapeutic agent into the middle and/or inner ear. Therapeutic agents include agents that have a therapeutic benefit in the ear. In certain embodiments, the matrix forming agent is a liquid at ambient conditions, which once administered to a subject, gels (e.g., becomes more viscous). In certain embodiments, the matrix forming agents gels upon mixing of two components of the composition. In some embodiments, each component comprises a matrix forming agent (e.g., two polysaccharide derivatives which undergo cross-linking upon mixing). In some embodiments, one component comprises the matrix forming agent, and the second component comprises an activator or catalyst which causes gelation when mixed with the matrix forming agent. In certain embodiments, the pharmaceutical composition does not substantially interfere with the hearing of the subject.

### Matrix Forming Agents

The matrix forming agent is a compound or mixture of compounds that forms a gel after administration. In certain embodiments, the matrix forming agent forms a gel after administration into a subject's ear canal. The gel composition acts a reservoir containing the therapeutic agent and permeation enhancer, allowing for sustained release of the therapeutic agent across a barrier (e.g., tympanic membrane). In certain embodiments, the gel maintains contact with the tympanic membrane. In some embodiments, the gel maintains contact for between 0.5 and 1 hours, between 1 and 4 hours, between 1 and 8 hours, between 1 and 16 hours, or between 1 and 24 hours. In some embodiments, the gel maintains contact for between 1 day and 3 days, between 1 and 7 days, or between 1 and 14 days. In some embodiments, the gel allows flux of the therapeutic agent across the tympanic membrane for between 0.5 and 1 hours, between 1 and 4 hours, between 1 and 8 hours, between 1 and 16 hours, or between 1 and 24 hours. In some embodiments, the gel maintains contact for between 1 day and 3 days, between 1 and 7 days, or between 1 and 14 days. Such a reservoir maintains contact with the tympanic membrane increasing the time for the therapeutic agent to cross the tympanic membrane and be delivered to the middle or inner ear. Such a reservoir maximizes exposure of the tympanic membrane to permeation enhancers and the therapeutic agent, and facilitates sustained flux of the therapeutic agent into the middle and inner ear.

In various embodiments, the composition is a sustained release formulation. In various aspects, sustained release of either the permeation enhancer and/or the therapeutic agent can be at a constant rate to deliver an effective amount of either the permeation enhancer or therapeutic agent to the surface of the tympanic membrane, the middle ear, or the inner ear. In various embodiments, the sustained release provides a sufficient flux of therapeutic agent over about 1 day, about 2 days, about 3 days, about 4 days, about 5 days, about 6 days, or about 7 days. In various embodiments, the sustained release provides a sufficient flux of therapeutic agent over a range of about 7 to about 10 days. In various embodiments, the sustained release may be at a constant rate over a range of about 7 days to about 14 days. In various embodiments, the sustained release provides a sufficient flux of therapeutic agent over a range of about 14 to about 21 days. In various embodiments, the sustained release provides a sufficient flux of therapeutic agent over a range of about 21 to about 30 days. As used herein, sufficient flux is the flux necessary for the therapeutic agent to be present in the middle ear in a therapeutically effective amount or prophylactically effective amount. In some embodiments, the sufficient flux is sufficient to provide an antibiotic agent in a concentration equal or greater to the minimum inhibitory concentration of an infectious microorganism. In some embodiments, the infectious microorganism is H. influenza, S. pneumoniae, or M. catarrhalis.

In various aspects, the sustained release profile is obtained by the addition of a matrix-forming agent to the composition. In various embodiments, the composition may further comprise a matrix forming agent. In various embodiments, the matrix forming agents may undergo a change in viscosity, in situ, based on a phase change, a change in solubility, evaporation of a solvent, or mixing of components comprising the matrix forming agent. Such matrix forming agents gel, in situ after administration into a patient's ear canal to form a reservoir containing the therapeutic agent and permeation enhancer, allowing sustained release of the therapeutic agent. Such a reservoir maintains contact with the tympanic membrane increasing the time for the therapeutic agent to permeate the tympanic membrane, and be delivered to the middle or inner ear. Such a reservoir maximizes exposure of the tympanic membrane to permeation enhancers and the therapeutic agent.

In certain embodiments, the matrix forming agent is a hydrogel, or forms a hydrogel upon administration. In certain embodiments, the matrix forming agent does not comprise a polymer. In certain embodiments, the matrix forming agent comprises a polymer that is poloxamer 407-poly(butoxy)phosphoester. In certain embodiments, the composition comprises between about 9.0-19.0% wt/vol of poloxamer 407-poly(butoxy)phosphoester. In certain embodiments, the composition comprises between about 10.0-15.0% wt/vol of poloxamer 407-poly(butoxy)phosphoester. In certain embodiments, the composition comprises between about 9.0-19.0% wt/vol, between about 9.0-17.0% wt/vol, between about 9.0-16.0% wt/vol, between about 10.0-17.0% wt/vol, between about 10.0-15.0% wt/vol, between about 10.0-14.0% wt/vol, between about 10.0-13.0% wt/vol, between about 10.0-12.0% wt/vol, between about 9.0-12.0% wt/vol, between about 9.0-11.0% wt/vol, or between about 9.0-10.0% wt/vol, of poloxamer 407-poly(butoxy)phosphoester. In certain embodiments, the composition comprises about 9.0% wt/vol, about 9.5% wt/vol, about 10.0% wt/vol, about 10.5% wt/vol, about 11.0% wt/vol, about 11.5% wt/vol, about 12.0% wt/vol, about 12.5% wt/vol, about 13.0% wt/vol, about 13.5% wt/vol, about 14.0% wt/vol, about 14.5% wt/vol, about 15.0% wt/vol, about 15.5% wt/vol, about 16.0% wt/vol, about 16.5% wt/vol, about 17.0% wt/vol, about 17.5% wt/vol, about 18.0% wt/vol, about 18.5% wt/vol, or about 19.0% wt/vol, of poloxamer 407-poly(butoxy)phosphoester. In certain embodiments, the composition comprises about 10.0% wt/vol of poloxamer 407-poly(butoxy)phosphoester. In certain embodiments, the composition comprises about 12.0% wt/vol of poloxamer 407-poly(butoxy)phosphoester. In certain embodiments, the composition comprises about 15.0% wt/vol of poloxamer 407-poly(butoxy)phosphoester.

In certain embodiments, the composition comprises between about 9.0-19.0% wt/vol of poloxamer 407-poly(butoxy)phosphoester. In certain embodiments, the composition comprises between about 9.0-20.0% wt/vol of poloxamer 407-poly(butoxy)phosphoester. In certain embodiments, the composition comprises between about 10.0-15.0% wt/vol of poloxamer 407-poly(butoxy)phosphoester. In certain embodiments, the composition comprises between about 9.0-10.0% wt/vol, between about 10.0-12.0% wt/vol, between about 12.0-13.0% wt/vol, between about 13.0-14.0% wt/vol, between about 14.0-15.0% wt/vol, between about 15.0-16.0% wt/vol, between about 16.0-17.0% wt/vol, between about 17.0-18.0% wt/vol, between about 18.0-19.0% wt/vol, between about 19.0-20.0% wt/vol, between about 20.0-21.0% wt/vol, between about 21.0-22.0% wt/vol, between about 22.0-23.0% wt/vol, between about 23.0-24.0% wt/vol, or between about 24.0-25.0% wt/vol, of poloxamer 407-poly(butoxy)phosphoester. In certain embodiments, the composition comprises about 9.0% wt/vol, about 9.5% wt/vol, about 10.0% wt/vol, about 10.5% wt/vol, about 11.0% wt/vol, about 11.5% wt/vol, about 12.0% wt/vol, about 12.5% wt/vol, about 13.0% wt/vol, about 13.5% wt/vol, about 14.0% wt/vol, about 14.5% wt/vol, about 15.0% wt/vol, about 15.5% wt/vol, about 16.0% wt/vol, about 16.5% wt/vol, about 17.0% wt/vol, about 17.5% wt/vol, about 18.0% wt/vol, about 18.5% wt/vol, about 19.0% wt/vol, about 19.5% wt/vol, or about 20.0% wt/vol, of poloxamer 407-poly(butoxy)phosphoester. In certain embodiments, the composition comprises about 10.0% wt/vol of poloxamer 407-poly(butoxy)phosphoester. In certain embodiments, the composition comprises about 12.0% wt/vol of poloxamer 407-poly(butoxy)phosphoester. In certain embodiments, the composition comprises about 15.0% wt/vol of poloxamer 407-poly(butoxy)phosphoester.

### Permeation Enhancers

A permeation enhancer refers to any agent that increases the flux of a therapeutic agent across a barrier (e.g., membrane, layer of cells). In some embodiments, the barrier is skin. In some embodiments, the barrier is the tympanic membrane. In some embodiments, the barrier is the tympanic membrane and not the nerve. In some embodiments, the barrier is not the nerve. In certain embodiments, the permeation enhancer is the surfactant sodium dodecyl sulfate. In certain embodiments, the permeation enhancer is the anesthetic bupivacaine. In certain embodiments, the permeation enhancer is the terpene limonene. In certain embodiments, the permeation enhancer comprises a single permeation enhancer. In certain embodiments, the permeation enhancer comprises the surfactant sodium dodecyl sulfate. In certain embodiments, the permeation enhancer comprises the anesthetic bupivacaine. In certain embodiments, the permeation enhancer comprises the terpene limonene. In certain embodiments, the permeation enhancer comprises a surfactant permeation enhancer. In certain embodiments, the permeation enhancer comprises an anesthetic permeation enhancer. In certain embodiments, the permeation enhancer comprises a terpene permeation enhancer. In certain embodiments, the permeation enhancer comprises two permeation enhancers. In certain embodiments, the permeation enhancer comprises a surfactant permeation enhancer and an anesthetic permeation enhancer. In certain embodiments, the permeation enhancer comprises a surfactant permeation enhancer and a terpene permeation enhancer. In certain embodiments, the permeation enhancer comprises an anesthetic permeation enhancer and a terpene permeation enhancer. In certain embodiments, the permeation enhancer comprises a surfactant permeation enhancer, an anesthetic permeation enhancer, and a terpene permeation enhancer. In certain embodiments, the permeation enhancer comprises three permeation enhancers. In certain embodiments, the permeation enhancer comprises the surfactant sodium dodecyl sulfate, the anesthetic bupivacaine, and the terpene limonene.

In certain embodiments, the composition comprises between about 0.5-5.5% wt/vol of a permeation enhancer that is sodium dodecyl sulfate. In certain embodiments, the composition comprises between about 0.5-5.5% wt/vol of sodium dodecyl sulfate, between about 0.75-5.5% wt/vol of sodium dodecyl sulfate, between about 1.0-5.25% wt/vol of sodium dodecyl sulfate, between about 1.25-5.25% wt/vol of sodium dodecyl sulfate, or between about 1.0-5.0% wt/vol of sodium dodecyl sulfate. In certain embodiments, the composition comprises between about 1.0-5.0% wt/vol of sodium dodecyl sulfate. In certain embodiments, the composition comprises about 0.5% wt/vol, about 0.75% wt/vol, about 1.0% wt/vol, about 1.25% wt/vol, about 1.5% wt/vol, about 1.75% wt/vol, about 2.0% wt/vol, about 2.25% wt/vol, about 2.5% wt/vol, about 2.75% wt/vol, about 3.0% wt/vol, about 3.25% wt/vol, about 3.5% wt/vol, about 3.75% wt/vol, about 4.0% wt/vol, about 4.25% wt/vol, about 4.5% wt/vol, about 4.75% wt/vol, about 5.0% wt/vol, or about 5.5% wt/vol, of sodium dodecyl sulfate. In certain embodiments, the composition comprises about 1.0% wt/vol of sodium dodecyl sulfate. In certain embodiments, the composition comprises about 2.0% wt/vol of sodium dodecyl sulfate. In certain embodiments, the composition comprises about 3.0% wt/vol of sodium dodecyl sulfate. In certain embodiments, the composition comprises about 4.0% wt/vol of sodium dodecyl sulfate. In certain embodiments, the composition comprises about 5.0% wt/vol of sodium dodecyl sulfate.

In certain embodiments, the composition comprises between about 0.5-20.0% wt/vol of a permeation enhancer that is sodium dodecyl sulfate. In certain embodiments, the composition comprises between about 0.5-10.0% wt/vol of a permeation enhancer that is sodium dodecyl sulfate. In certain embodiments, the composition comprises between about 10.0-20.0% wt/vol of a permeation enhancer that is sodium dodecyl sulfate. In certain embodiments, the composition comprises between about 12.0-20.0% wt/vol of a permeation enhancer that is sodium dodecyl sulfate. In certain embodiments, the composition comprises between about 10.0-20.0% wt/vol of a permeation enhancer that is sodium dodecyl sulfate. In certain embodiments, the composition comprises between about 12.0-15.0% wt/vol of a permeation enhancer that is sodium dodecyl sulfate. In certain embodiments, the composition comprises between about 0.5-5.0% wt/vol of a permeation enhancer that is sodium dodecyl sulfate. In certain embodiments, the composition comprises between about 1.0-5.0% wt/vol of a permeation enhancer that is sodium dodecyl sulfate. In certain embodiments, the composition comprises about 0.5% wt/vol, about 0.75% wt/vol, about 1.0% wt/vol, about 1.25% wt/vol, about 1.5% wt/vol, about 1.75% wt/vol, about 2.0% wt/vol, about 2.25% wt/vol, about 2.5% wt/vol, about 2.75% wt/vol, about 3.0% wt/vol, about 3.25% wt/vol, about 3.5% wt/vol, about 3.75% wt/vol, about 4.0% wt/vol, about 4.25% wt/vol, about 4.5% wt/vol, about 4.75% wt/vol, about 5.0% wt/vol, about 5.5% wt/vol, about 6.0% wt/vol, about 6.5% wt/vol, about 7.0% wt/vol, about 7.5% wt/vol, about 8.0% wt/vol, about 8.5% wt/vol, about 9.0% wt/vol, about 9.5% wt/vol, about 10.0% wt/vol, about 10.5% wt/vol, about 11.0% wt/vol, about 11.5% wt/vol, about 12.0% wt/vol, about 12.5% wt/vol, about 13.0% wt/vol, about 13.5% wt/vol, about 14.0% wt/vol, about 14.5% wt/vol, about 15.0% wt/vol, about 15.5% wt/vol, about 16.0% wt/vol, about 16.5% wt/vol, about 17.0% wt/vol, about 17.5% wt/vol, about 18.0% wt/vol, about 18.5% wt/vol, about 19.0% wt/vol, about 19.5% wt/vol, about 20.0% wt/vol, or about 25.5% wt/vol, of sodium dodecyl sulfate.

In certain embodiments, the composition comprises about 0.5% wt/vol to about 5.0% wt/vol of a permeation enhancer that is sodium dodecyl sulfate, about 5.0% wt/vol to about 10.0% wt/vol of a permeation enhancer that is sodium dodecyl sulfate, about 10.0% wt/vol to about 15.0% wt/vol of a permeation enhancer that is sodium dodecyl sulfate, about 15.0% wt/vol to about 20.0% wt/vol of a permeation enhancer that is sodium dodecyl sulfate, about 20.0% wt/vol to about 22.5% wt/vol of a permeation enhancer that is sodium dodecyl sulfate, about 22.5% wt/vol to about 25.0% wt/vol of a permeation enhancer that is sodium dodecyl sulfate, about 20.0% wt/vol to about 25.0% wt/vol of a permeation enhancer that is sodium dodecyl sulfate, or about 25.0% wt/vol to about 27.5% wt/vol of a permeation enhancer that is sodium dodecyl sulfate.

In certain embodiments, the composition comprises between about 0.5-1.5% wt/vol, between about 0.75-1.5% wt/vol, between about 1.0-1.5% wt/vol, or between about 1.25-1.5% wt/vol, of a permeation enhancer that is bupivacaine. In certain embodiments, the composition comprises between about 0.5-1.5% wt/vol of a permeation enhancer that is bupivacaine. In certain embodiments, the composition comprises about 0.5% wt/vol, about 0.75% wt/vol, about 1.0% wt/vol, about 1.25% wt/vol, or about 1.5% wt/vol, of bupivacaine. In certain embodiments, the composition comprises about 0.5% wt/vol of bupivacaine. In certain embodiments, the composition comprises about 0.75% wt/vol of bupivacaine. In certain embodiments, the composition comprises about 1.0% wt/vol of bupivacaine. In certain embodiments, the composition comprises about 1.25% wt/vol of bupivacaine.

In certain embodiments, the composition comprises between about 0.5-7.5% wt/vol, between about 0.5-2.5% wt/vol, between about 0.75-2.5% wt/vol, between about 1.0-2.5% wt/vol, between about 1.25-2.5% wt/vol, between about 1.75-7.5% wt/vol, between about 2.5-5.5% wt/vol, between about 2.5-7.5% wt/vol, between about 5.5-7.0% wt/vol, or between about 2.5-7.5% wt/vol, of a permeation enhancer that is bupivacaine. In certain embodiments, the composition comprises between about 0.5-2.5% wt/vol of a permeation enhancer that is bupivacaine. In certain embodiments, the composition comprises about 0.5% wt/vol, about 0.75% wt/vol, about 1.0% wt/vol, about 1.25% wt/vol, about 1.5% wt/vol, about 2.0% wt/vol, about 2.25% wt/vol, about 2.5% wt/vol, about 3.0% wt/vol, about 3.5% wt/vol, about 4.0% wt/vol, about 4.5% wt/vol, about 5.0% wt/vol, about 5.5% wt/vol, about 6.0% wt/vol, about 6.5% wt/vol, about 7.0% wt/vol, or about 7.5% wt/vol, of bupivacaine. In certain embodiments, the composition comprises between about 1.75-7.5% wt/vol of bupivacaine. In certain embodiments, the composition comprises between about 2.0-7.5% wt/vol of bupivacaine. In certain embodiments, the composition comprises about 0.5% wt/vol of bupivacaine. In certain embodiments, the composition comprises about 0.75% wt/vol of bupivacaine. In certain embodiments, the composition comprises about 1.0% wt/vol of bupivacaine. In certain embodiments, the composition comprises about 1.25% wt/vol of bupivacaine. In certain embodiments, the composition comprises about 1.5% wt/vol of bupivacaine. In certain embodiments, the composition comprises about 1.75% wt/vol of bupivacaine. In certain embodiments, the composition comprises about 2.0% wt/vol of bupivacaine. In certain embodiments, the composition comprises about 2.25% wt/vol of bupivacaine. In certain embodiments, the composition comprises about 2.5% wt/vol of bupivacaine. In certain embodiments, the composition comprises about 3.0% wt/vol of bupivacaine. In certain embodiments, the composition comprises about 3.5% wt/vol of bupivacaine. In certain embodiments, the composition comprises about 4.0% wt/vol of bupivacaine. In certain embodiments, the composition comprises about 4.5% wt/vol of bupivacaine. In certain embodiments, the composition comprises about 5.0% wt/vol of bupivacaine. In certain embodiments, the composition comprises about 5.5% wt/vol of bupivacaine. In certain embodiments, the composition comprises about 6.0% wt/vol of bupivacaine. In certain embodiments, the composition comprises about 6.5% wt/vol of bupivacaine. In certain embodiments, the composition comprises about 7.0% wt/vol of bupivacaine. In certain embodiments, the composition comprises about 7.5% wt/vol of bupivacaine. In certain embodiments, the composition does not comprise between 8.0-15.0% wt/vol or between 8.5-15.0% wt/vol of bupivacaine.

In certain embodiments, the composition comprises between about 0.5-0.75% wt/vol of a permeation enhancer that is bupivacaine, between about 0.75-1.0% wt/vol of a permeation enhancer that is bupivacaine, between about 1.0-1.25% wt/vol of a permeation enhancer that is bupivacaine, between about 1.25-1.5% wt/vol of a permeation enhancer that is bupivacaine, between about 1.5-1.75% wt/vol of a permeation enhancer that is bupivacaine, between about 1.75-2.25% wt/vol of a permeation enhancer that is bupivacaine, between about 2.25-2.5% wt/vol of a permeation enhancer that is bupivacaine, between about 2.25-2.5% wt/vol of a permeation enhancer that is bupivacaine, between about 2.5-3.0% wt/vol of a permeation enhancer that is bupivacaine, between about 3.0-4.0% wt/vol of a permeation enhancer that is bupivacaine, between about 4.0-5.0% wt/vol of a permeation enhancer that is bupivacaine, between about 5.0-6.0% wt/vol of a permeation enhancer that is bupivacaine, between about 6.0-7.0% wt/vol of a permeation enhancer that is bupivacaine, between about 6.0-7.5% wt/vol of a permeation enhancer that is bupivacaine, or between about 2.5-7.5% wt/vol of a permeation enhancer that is bupivacaine, of a permeation enhancer that is bupivacaine.

In certain embodiments, the composition comprises between about 0.5-10.0% wt/vol of a permeation enhancer that is limonene. In certain embodiments, the composition comprises between about 0.5-12.0% wt/vol of a permeation enhancer that is limonene. In certain embodiments, the composition comprises between about 1.5-12.0% wt/vol of a permeation enhancer that is limonene. In certain embodiments, the composition comprises between about 1.5-10.0% wt/vol of a permeation enhancer that is limonene. In certain embodiments, the composition comprises between about 0.5-3.5% wt/vol of a permeation enhancer that is limonene. In certain embodiments, the composition comprises between about 0.5-3.5% wt/vol, between about 1.5-5.0% wt/vol, between about 1.5-4.75% wt/vol, between about 1.5-4.5% wt/vol, between about 1.5-4.25% wt/vol, between about 1.5-4.0% wt/vol, between about 1.5-3.75% wt/vol, between about 1.5-3.5% wt/vol, between about 1.5-3.25% wt/vol, between about 1.5-3.0% wt/vol, between about 1.5-2.75% wt/vol, between about 1.5-2.5% wt/vol, between about 1.5-2.25% wt/vol, between about 1.5-2.0% wt/vol, between about 1.25-2.25% wt/vol, or between about 1.0-2.5% wt/vol. In certain embodiments, the composition comprises about 2.0% wt/vol of limonene.

In certain embodiments, the composition comprises between about 2.0-12.0% wt/vol of a permeation enhancer that is limonene. In certain embodiments, the composition comprises between about 1.5-12.0% wt/vol, between about 1.5-11.5% wt/vol, between about 1.5-11.0% wt/vol, between about 1.5-10.0% wt/vol, between about 1.5-9.0% wt/vol, between about 1.5-8.0% wt/vol, between about 2.0-9.0% wt/vol, between about 2.0-10.0% wt/vol, between about 3.0-11.0% wt/vol, between about 4.0-10.0% wt/vol, of a permeation enhancer that is limonene. In certain embodiments, the composition comprises about 2.0% wt/vol, about 2.25% wt/vol, about 2.5% wt/vol, about 2.75% wt/vol, about 3.0% wt/vol, about 3.25% wt/vol, about 3.5% wt/vol, about 3.75% wt/vol, about 4.0% wt/vol, about 4.5% wt/vol, about 5.0% wt/vol, about 5.5% wt/vol, about 6.0% wt/vol, about 6.5% wt/vol, about 7.0% wt/vol, about 7.5% wt/vol, about 8.0% wt/vol, about 8.5% wt/vol, about 9.0% wt/vol, about 9.5% wt/vol, about 10.0% wt/vol, about 10.5% wt/vol, about 11.0% wt/vol, about 11.5% wt/vol, or about 12.0% wt/vol, of limonene. In certain embodiments, the composition comprises about 2.0% wt/vol of limonene. In certain embodiments, the composition comprises about 3.0% wt/vol of limonene. In certain embodiments, the composition comprises about 4.0% wt/vol of limonene. In certain embodiments, the composition comprises about 5.0% wt/vol of limonene. In certain embodiments, the composition comprises about 6.0% wt/vol of limonene. In certain embodiments, the composition comprises about 7.0% wt/vol of limonene. In certain embodiments, the composition comprises about 8.0% wt/vol of limonene. In certain embodiments, the composition comprises about 9.0% wt/vol of limonene.

In certain embodiments, the composition comprises about 10.0% wt/vol of limonene.

In certain embodiments, the composition comprises between about 1.5-15.0% wt/vol, between about 1.5-3.0% wt/vol, between about 3.0-5.0% wt/vol, between about 5.0-7.0% wt/vol, between about 7.0-9.0% wt/vol, between about 7.0-11.0% wt/vol, between about 9.0-13.0% wt/vol, between about 11.0-13.0% wt/vol, between about 13.0-14.0% wt/vol, between about 14.0-15.0% wt/vol, between about 8.0-12.5.0% wt/vol, or between about 8.0-15.0% wt/vol, of a permeation enhancer that is limonene. In certain embodiments, the composition comprises about 2.0% wt/vol, about 2.25% wt/vol, about 2.5% wt/vol, about 2.75% wt/vol, about 3.0% wt/vol, about 3.25% wt/vol, about 3.5% wt/vol, about 3.75% wt/vol, about 4.0% wt/vol, about 4.5% wt/vol, about 5.0% wt/vol, about 5.5% wt/vol, about 6.0% wt/vol, about 6.5% wt/vol, about 7.0% wt/vol, about 7.5% wt/vol, about 8.0% wt/vol, about 8.5% wt/vol, about 9.0% wt/vol, about 9.5% wt/vol, about 10.0% wt/vol, about 10.5% wt/vol, about 11.0% wt/vol, about 11.5% wt/vol, about 12.0% wt/vol, about 13.0% wt/vol, about 14.0% wt/vol, or about 15.0% wt/vol, of limonene. In certain embodiments, the composition comprises about 2.0% wt/vol of limonene. In certain embodiments, the composition comprises about 3.0% wt/vol of limonene. In certain embodiments, the composition comprises about 4.0% wt/vol of limonene. In certain embodiments, the composition comprises about 5.0% wt/vol of limonene. In certain embodiments, the composition comprises about 6.0% wt/vol of limonene. In certain embodiments, the composition comprises about 7.0% wt/vol of limonene. In certain embodiments, the composition comprises about 8.0% wt/vol of limonene. In certain embodiments, the composition comprises about 9.0% wt/vol of limonene. In certain embodiments, the composition comprises about 10.0% wt/vol of limonene. In certain embodiments, the composition comprises about 11.0% wt/vol of limonene. In certain embodiments, the composition comprises about 12.0% wt/vol of limonene. In certain embodiments, the composition comprises about 13.0% wt/vol of limonene. In certain embodiments, the composition comprises about 14.0% wt/vol of limonene. In certain embodiments, the composition comprises about 15.0% wt/vol of limonene.

In certain embodiments, the composition comprises: between about 1.0-5.0% wt/vol of sodium dodecyl sulfate; between about 0.5-1.0% wt/vol of bupivacaine; between about 4.0-10.0% wt/vol of limonene; and between about 12.0-15.0% wt/vol of poloxamer 407-poly(butoxy)phosphoester.

In certain embodiments, the composition comprises between about 0.5-5.0% wt/vol of sodium dodecyl sulfate; between about 0.5-7.5% wt/vol of bupivacaine; between about 0.5-3.5% wt/vol of limonene; between about 9.0-15.0% wt/vol of poloxamer 407-poly(butoxy)phosphoester; and between about 0.01-0.50% wt/vol of another therapeutic agent that is a sodium channel blocker anesthetic agent of tetrodotoxin.

In certain embodiments, the composition comprises:
(a) a therapeutic agent or a combination of therapeutic agents (e.g., an antibiotic (e.g., ciproflaxin)); (b) a permeation enhancer or a combination of permeation enhancers, wherein the permeation enhancer or combination of permeation enhancers increases the flux of the therapeutic agent or combination of therapeutic agents across a barrier; and (c) a matrix forming agent or a combination of matrix forming agents, wherein the matrix forming agent or combination of matrix forming agents comprises a polymer; wherein: the composition forms a gel at temperatures above a phase transition temperature; and
the phase transition temperature is less than about 37° C.; wherein the composition comprises between about 0.5-5.5% wt/vol of a permeation enhancer that is sodium dodecyl sulfate; wherein the composition comprises between about 0.5-1.5% wt/vol of a permeation enhancer that is bupivacaine; wherein the composition comprises between about 2.0-12.0% wt/vol of a permeation enhancer that is limonene; and wherein the composition comprises between about 9.0-20.0% wt/vol of a polymer that is poloxamer 407-poly(butoxy)phosphoester.

In certain embodiments, the composition comprises:
(a) a therapeutic agent or a combination of therapeutic agents (e.g., an antibiotic (e.g., ciproflaxin)); (b) a permeation enhancer or a combination of permeation enhancers, wherein the permeation enhancer or combination of permeation enhancers increases the flux of the therapeutic agent or combination of therapeutic agents across a barrier; and (c) a matrix forming agent or a combination of matrix forming agents, wherein the matrix forming agent or combination of matrix forming agents comprises a polymer;
   wherein: the composition forms a gel at temperatures above a phase transition temperature; and the phase transition temperature is less than about 37° C.; wherein the composition comprises between about 1.0-5.25% wt/vol of a permeation enhancer that is sodium dodecyl sulfate; wherein the composition comprises between about 0.5-1.25% wt/vol of a permeation enhancer that is bupivacaine; wherein the composition comprises between about 1.5-11.5% wt/vol of a permeation enhancer that is limonene; and wherein the composition comprises between about 9.5-19.5% wt/vol of a polymer that is poloxamer 407-poly(butoxy)phosphoester.

In certain embodiments, the composition comprises: either:

(1) about 1.0% wt/vol of sodium dodecyl sulfate; about 0.5% wt/vol of bupivacaine; about 2.0% wt/vol of limonene; and about 12.0% wt/vol of poloxamer 407-poly(butoxy)phosphoester;
(2) about 1.0% wt/vol of sodium dodecyl sulfate; about 1.0% wt/vol of bupivacaine; about 10.0% wt/vol of limonene; and about 12.0% wt/vol of poloxamer 407-poly(butoxy)phosphoester;
(3) about 1.0% wt/vol of sodium dodecyl sulfate; about 1.0% wt/vol of bupivacaine; about 10.0% wt/vol of limonene; and about 15.0% wt/vol of poloxamer 407-poly(butoxy)phosphoester;
(4) about 5.0% wt/vol of sodium dodecyl sulfate; about 1.0% wt/vol of bupivacaine; about 4.0% wt/vol of limonene; and about 12.0% wt/vol of poloxamer 407-poly(butoxy)phosphoester; or
(5) about 5.0% wt/vol of sodium dodecyl sulfate; about 1.0% wt/vol of bupivacaine; about 4.0% wt/vol of limonene; and about 15.0% wt/vol of poloxamer 407-poly(butoxy)phosphoester.

In certain embodiments, the composition comprises:

(1) about 1.0% wt/vol of sodium dodecyl sulfate; about 0.5% wt/vol of bupivacaine; about 2.0% wt/vol of limonene; and about 12.0% wt/vol of poloxamer 407-poly(butoxy)phosphoester. In certain embodiments, the composition comprises: (2) about 1.0% wt/vol of sodium dodecyl sulfate; about 1.0% wt/vol of bupivacaine; about 10.0% wt/vol of limonene; and about 12.0% wt/vol of poloxamer 407-poly(butoxy)phosphoester. In certain embodiments, the composition comprises: (3) about 1.0% wt/vol of sodium dodecyl sulfate; about 1.0% wt/vol of bupivacaine; about 10.0% wt/vol of limonene; and about 15.0% wt/vol of poloxamer 407-poly(butoxy)phosphoester. In certain embodiments, the composition comprises: (4) about 5.0% wt/vol of sodium dodecyl sulfate; about 1.0% wt/vol of bupivacaine; about 4.0% wt/vol of limonene; and about 12.0% wt/vol of poloxamer 407-poly(butoxy)phosphoester. In certain embodiments, the composition comprises: (5) about 5.0% wt/vol of sodium dodecyl sulfate; about 1.0% wt/vol of bupivacaine; about 4.0% wt/vol of limonene; and about 15.0% wt/vol of poloxamer 407-poly(butoxy)phosphoester.

In certain embodiments, the composition comprises: about 1.0% wt/vol of sodium dodecyl sulfate; about 2.0% wt/vol of bupivacaine; about 2.0% wt/vol of limonene; about 12.0% wt/vol of poloxamer 407-poly(butoxy)phosphoester; and about 0.03% wt/vol of another therapeutic agent that is a sodium channel blocker anesthetic agent of tetrodotoxin. In certain embodiments, the composition comprises: about 1.0% wt/vol of sodium dodecyl sulfate; about 2.0% wt/vol of bupivacaine; about 2.0% wt/vol of limonene; about 12.0% wt/vol of poloxamer 407-poly(butoxy)phosphoester; and about 0.3% wt/vol of another therapeutic agent that is a sodium channel blocker anesthetic agent of tetrodotoxin.

### Therapeutic Agents

A therapeutic agent can be any agent used to treat any ear disease, or symptom of an ear disease or infectious disease (e.g., pain associated with an ear disease or infectious disease). A therapeutic agent can be an agent used to treat pain. Therapeutic agents may include antimicrobial agents. Therapeutic agents may include, but are not limited to, antimicrobial agents, antibiotics, anesthetics, anti-inflammatories, analgesics, anti-fibrotics, anti-sclerotics, and anticoagulants. Therapeutic agents may include, but are not limited to, antibiotics, anesthetics, anti-inflammatories, analgesics, anti-fibrotics, anti-sclerotics, and anticoagulants. In certain embodiments, the therapeutic agent is an antimicrobial agent. In certain embodiments, the therapeutic agent is an antibiotic agent. In certain embodiments, the therapeutic agent is an anesthetic agent. In certain embodiments, the therapeutic agent is an anti-inflammatory agent. In certain embodiments, the therapeutic agent is an analgesic agent. In certain embodiments, the therapeutic agent is an anti-fibrotic agent. In certain embodiments, the therapeutic agent is an anti-sclerotic agent. In certain embodiments, the therapeutic agent is an anticoagulant agent.

In various aspects, the therapeutic agents may comprise between about 0.01 percent to about 10 percent of the composition. In various embodiments, the therapeutic agents may comprise between about 0.01 percent to about 1 percent of the composition, comprise between about 1 percent to about 2 percent of the composition, comprise between about 2 percent to about 3 percent of the composition, comprise between about 3 percent to about 4 percent of the composition, comprise between about 4 percent to about 5 percent of the composition, comprise between about 5 percent to about 6 percent of the composition, comprise between about 6 percent to about 7 percent of the composition, comprise between about 7 percent to about 8 percent of the composition, comprise between about 8 percent to about 9 percent of the composition, or comprise between about 9 percent to about 10 percent of the composition.

In various aspects, the therapeutic agents may comprise between about 0.01 percent to about 10 percent wt/vol of the composition. In various aspects, the therapeutic agents may comprise between about 1.0 percent to about 7.0 percent wt/vol of the composition. In various aspects, the therapeutic agents may comprise between about 1.0 percent to about 6.0 percent wt/vol of the composition.

The exact amount required will vary from subject to subject, depending on the species, age, and general condition of the subject, the particular compound, its mode of administration, its mode of activity, condition being treated, and the like. The compositions described herein are preferably formulated in dosage unit form for ease of administration and uniformity of dosage. It will be understood, however, that the total daily usage of the compounds and compositions will be decided by the attending physician within the scope of sound medical judgment. The specific therapeutically effective dose level for any particular patient or organism will depend upon a variety of factors including the disorder being treated and the severity of the disorder; the activity of the specific compound employed; the specific composition employed; the age, body weight, general health, sex and diet of the patient; the time of administration, route of administration, and rate of excretion of the specific compound employed; the duration of the treatment; drugs used in combination or coincidental with the specific compound employed; and like factors well known in the medical arts.

In certain embodiments, the therapeutic agent is an antimicrobial agent. In certain embodiments, the therapeutic agent is an antibiotic. Any antibiotic may be used in the system. In certain embodiments the antibiotic is approved for use in humans or other animals. In certain embodiments the antibiotic is approved for use by the U.S. Food & Drug Administration. In certain embodiments, the antibiotic may be selected from the group consisting of cephalosporins, quinolones, polypeptides, macrolides, penicillins, and sulfonamides. Exemplary antibiotics may include, but are not limited to, ciprofloxacin, cefuroxime, cefadroxil, cefazolin, cefalotin, cefalexin, cefaclor, cefamandole, cefoxitin, cefprozil, cefuroxime, cefixime, cefdinir, cefditoren, cefoperazone, cefotaxime, cefpodoxime, ceftazidime, ceftibuten, ceftizoxime, ceftriaxone, cefepime, ceftobiprole, enoxacin, gatifloxacin, levofloxacin, lomefloxacin, moxifloxacin, norfloxacin, ofloxacin, trovafloxacin, bacitracin, colistin, polymyxin B, azithromycin, clarithromycin, dirithromycin, erythromycin, roxithromycin, troleandomycin, telithromycin, spectinomycin, amoxicillin, ampicillin, azlocillin, carbenicillin, cloxacillin, dicloxacillin, flucloxacillin, mezlocillin, meticillin, nafcillin, oxacillin, penicillin, piperacillin, ticarcillin, mafenide, sulfacetamide, sulfamethizole, sulfasalazine, sulfisoxazole, trimethoprim, and trimethoprim-sulfamethoxazole.

In certain embodiments, the therapeutic agent is an antibiotic agent, anesthetic agent, anti-inflammatory agent, analgesic agent, anti-fibrotic agent, anti-sclerotic agent, anticoagulant agent, or diagnostic agent.

In certain embodiments, the antibiotic is a quinolone. In certain embodiments, the antibiotic is a carbapenem. In certain embodiments, the antibiotic is amoxicillin, azithromicicn, cefuroxime, ceftriaxone, trimethoprim, levofloxacin, moxifloxacin, meropenem, or ciprofloxacin. In some embodiments, the antibiotic is ciprofloxacin. In some embodiments, the antibiotic is ciprofloxacin and pharmaceutically acceptable salts thereof. In some embodiments, the antibiotic is ciprofloxacin hydrochloride. In some embodiments, the antibiotic is levofloxacin.

Exemplary antibiotics, include, but are not limited to: Abamectin, Actinomycin (e.g., Actinomycin A, Actinomycin C, Actinomycin D, Aurantin), Alatrofloxacin mesylate, Amikacin sulfate, Aminosalicylic acid, Anthracyclines (e.g., Aclarubicin, Adriamycin, Doxorubicin, Epirubicin, Idarubicin), Antimycin (e.g., Antimycin A), Avermectin, BAL 30072, Bacitracin, Bleomycin, Cephalosporins (e.g., 7-Aminocephalosporanic acid, 7-Aminodeacetoxycephalosporanic acid, Cefaclor, Cefadroxil, Cefamandole, Cefazolin, Cefepime, Cefixime, Cefmenoxime, Cefmetazole, Cefoperazone, Cefotaxime, Cefotetan, Cefotiam, Cefoxitin, Cefpirome, Cefpodoxime proxetil, Cefsulodin, Cefsulodin sodium, Ceftazidime, Ceftizoxime, Ceftriaxone, Cefuroxime, Cephalexin, Cephaloridine, Cephalosporin C, Cephalothin, Cephalothin sodium, Cephapirin, Cephradine), Ciprofloxacin, Enrofloxacin, Clarithromycin, Clavulanic acid, Clindamycin, Colicin, Cyclosporin (e.g. Cyclosporin A), Dalfopristin/quinupristin, Daunorubicin, Doxorubicin, Epirubicin, GSK 1322322, Geneticin, Gentamicin, Gentamicin sulfate, Gramicidin (e.g. Gramicidin A), Grepafloxacin hydrochloride, Ivermectin, Kanamycin (e.g. Kanamycin A), Lasalocid, Leucomycin, Levofloxacin, Linezolid, Lomefloxacin, Lovastatin, MK 7655, Meropenem, Mevastatin, Mithramycin, Mitomycin, Monomycin, Natamycin, Neocarzinostatin, Neomycin (e.g. Neomycin sulfate), Nystatin, Oligomycin, Olivomycin, Pefloxacin, Penicillin (e.g. 6-Aminopenicillanic acid, Amoxicillin, Amoxicillin-clavulanic acid, Ampicillin, Ampicillin sodium, Azlocillin, Carbenicillin, Cefoxitin, Cephaloridine, Cloxacillin, Dicloxacillin, Mecillinam, Methicillin, Mezlocillin, Nafcillin, Oxacillin, Penicillin G, Penicillin G potassium, Penicillin G procaine, Penicillin G sodium, Penicillin V, Piperacillin, Piperacillin-tazobactam, Sulbactam, Tazobactam, Ticarcillin), Phleomycin, Polymyxin (e.g., Colistin, Polymyxin B), Pyocin (e.g. Pyocin R), RPX 7009, Rapamycin, Ristocetin, Salinomycin, Sparfloxacin, Spectinomycin, Spiramycin, Streptogramin, Streptovaricin, Tedizolid phosphate, Teicoplanin, Telithromycin, Tetracyclines (e.g. Achromycin V, Demeclocycline, Doxycycline, Doxycycline monohydrate, Minocycline, Oxytetracycline, Oxytetracycline hydrochloride Tetracycline, Tetracycline hydrochloride), Trichostatin A, Trovafloxacin, Tunicamycin, Tyrocidine, Valinomycin, (−)-Florfenicol, Acetylsulfisoxazole, Actinonin, Amikacin sulfate, Benzethonium chloride, Cetrimide, Chelerythrine, Chlorhexidine (e.g., Chlorhexidine gluconate), Chlorhexidine acetate, Chlorhexidine gluconate, Chlorothalonil, Co-Trimoxazole, Dichlorophene, Didecyldimethylammonium chloride, Dihydrostreptomycin, Enoxacin, Ethambutol, Fleroxacin, Furazolidone, Methylisothiazolinone, Monolaurin, Oxolinic acid, Povidone-iodine, Spirocheticides (e.g., Arsphenamine, Neoarsphenamine), Sulfaquinoxaline, Thiamphenicol, Tinidazole, Triclosan, Trovafloxacin, Tuberculostatics (e.g., 4-Aminosalicylic acid, AZD 5847, Aminosalicylic acid, Ethionamide), Vidarabine, Zinc pyrithione, and Zirconium phosphate.

In certain embodiments, the therapeutic agent is a Food and Drug Administration (FDA) approved drug for treating infections or infectious diseases. Exemplary FDA approved agents include, but are not limited to: Avycaz (ceftazidime-avibactam), Cresemba (isavuconazonium sulfate), Evotaz (atazanavir and cobicistat, Prezcobix (darunavir and cobicistat), Dalvance (dalbavancin), Harvoni (ledipasvir and sofosbuvir), Impavido (miltefosine), Jublia (efinaconazole), Kerydin (tavaborole), Metronidazole, Orbactiv (oritavancin), Rapivab (peramivir injection), Sivextro (tedizolid phosphate), Triumeq (abacavir, dolutegravir, and lamivudine), Viekira Pak (ombitasvir, paritaprevir, ritonavir and dasabuvir), Xtoro (finafloxacin), Zerbaxa (ceftolozane+tazobactam), Luzu (luliconazole), Olysio (simeprevir), Sitavig (acyclovir), Sovaldi (sofosbuvir), Abthrax (raxibacumab), Afinitor (everolimus), Cystaran (cysteamine hydrochloride), Dymista (azelastine hydrochloride and fluticasone propionate), Fulyzaq (crofelemer), Jetrea (ocriplasmin), Linzess (linaclotide), Qnasl (beclomethasone dipropionate) nasal aerosol, Sirturo (bedaquiline), Sklice (ivermectin), Stribild (elvitegravir, cobicistat, emtricitabine, tenofovir disoproxil fumarate), Tudorza Pressair (aclidinium bromide inhalation powder), Complera (emtricitabine/rilpivirine/tenofovir disoproxil fumarate), Dificid (fidaxomicin), Edurant (rilpivirine), Eylea (aflibercept), Firazyr (icatibant), Gralise (gabapentin), Incivek (telaprevir), Victrelis (boceprevir), Egrifta (tesamorelin), Teflaro (ceftaroline fosamil), Zymaxid (gatifloxacin), Bepreve (bepotastine besilate), Vibativ (telavancin), Aptivus (tipranavir), Astepro (azelastine hydrochloride nasal spray), Intelence (etravirine), Patanase (olopatadine hydrochloride), Viread (tenofovir disoproxil fumarate), Isentress (raltegravir), Selzentry (maraviroc), Veramyst (fluticasone furoate), Xyzal (levocetirizine dihydrochloride), Eraxis (anidulafungin), Noxafil (posaconazole), Prezista (darunavir), Tyzeka (telbivudine), Veregen (kunecatechins), Baraclude (entecavir), Fuzeon (enfuvirtide), Lexiva (fosamprenavir calcium), Reyataz (atazanavir sulfate), Clarinex, Hepsera (adefovir dipivoxil), Pegasys (peginterferon alfa-2a), Sustiva, Vfend (voriconazole), Zelnorm (tegaserod maleate), Avelox (moxifloxacin hydrochloride), Cancidas, Invanz, Peg-Intron (peginterferon alfa-2b), Rebetol (ribavirin), Spectracef, Tavist (clemastine fumarate), Twinrix, Valcyte (valganciclovir HCl), Xigris (drotrecogin alfa), ABREVA (docosanol), Cefazolin, Kaletra, Lamisil (terbinafine hydrochloride), Lotrisone (clotrimazole/betamethasone diproprionate), Lotronex (alosetron HCL), Trizivir (abacavir sulfate, lamivudine, zidovudine AZT), Synercid, Synagis, Viroptic, Aldara (imiquimod), Bactroban, Ceftin (cefuroxime axetil), Combivir, Condylox (pokofilox), Famvir (famciclovir), Floxin, Fortovase, INFERGEN (interferon alfacon-1), Intron A (interferon alfa-2b, recombinant), Mentax (butenafine HCl), Norvir (ritonavir), Omnicef, Rescriptor (delavirdine mesylate), Taxol, Timentin, Trovan, VIRACEPT (nelfinavir mesylate), Zerit (stavudine), AK-Con-A (naphazoline ophthalmic), Allegra (fexofenadine hydrochloride), Astelin nasal spray, Atrovent (ipratropium bromide), Augmentin (amoxicillin/clavulanate), Crixivan (Indinavir sulfate), Elmiron (pentosan polysulfate sodium), Havrix, Leukine (sargramostim), Merrem (meropenem), Nasacort AQ (triamcinolone acetonide), Tavist (clemastine fumarate), Vancenase AQ, Videx (didanosine), Viramune (nevirapine), Zithromax (azithromycin), Cedax (ceftibuten), Clarithromycin (Biaxin), Epivir (lamivudine), Invirase (saquinavir), Valtrex (valacyclovir HCl), Zyrtec (cetirizine HCl), Acyclovir, Penicillin (penicillin g potassium), Cubicin (Daptomycin), Factive (Gemifloxacin), Albenza (albendazole), Alinia (nitazoxanide), Altabax (retapamulin), AzaSite (azithromycin), Besivance (besifloxacin ophthalmic suspension), Biaxin XL (clarithromycin extended-release), Cayston (aztreonam), Cleocin (clindamycin phosphate), Doribax (doripenem), Dynabac, Flagyl ER, Ketek (telithromycin), Moxatag (amoxicillin), Rapamune (sirolimus), Restasis (cyclosporine), Tindamax (tinidazole), Tygacil (tigecycline), and Xifaxan (rifaximin). In certain embodiments, the antibiotic agent is selected from the group consisting of ciprofloxacin, cefuroxime, cefadroxil, cefazolin, cefalotin, cefalexin, cefaclor, cefamandole, cefoxitin, cefprozil, cefuroxime, cefixime, cefdinir, cefditoren, cefoperazone, cefotaxime, cefpodoxime, ceftazidime, ceftibuten, ceftizoxime, ceftriaxone, cefepime, ceftobiprole, enoxacin, gatifloxacin, levofloxacin, lomefloxacin, moxifloxacin, norfloxacin, ofloxacin, trovafloxacin, bacitracin, colistin, polymyxin B, azithromycin, clarithromycin, dirithromycin, erythromycin, roxithromycin, troleandomycin, telithromycin, spectinomycin, amoxicillin, ampicillin, azlocillin, carbenicillin, cloxacillin, dicloxacillin, flucloxacillin, mezlocillin, meticillin, nafcillin, oxacillin, penicillin, piperacillin, ticarcillin, mafenide, sulfacetamide, sulfamethizole, sulfasalazine, sulfisoxazole, trimethoprim, and trimethoprim-sulfamethoxazole. In certain embodiments, the antibiotic agent is ciprofloxacin. In certain embodiments, the composition comprises between about 1.0-5.0% wt/vol of ciprofloxacin.

In certain embodiments, the therapeutic agent is an anesthetic. Any anesthetic may be used in the system. In certain embodiments the anesthetic is approved for use in humans or other animals. In certain embodiments the anesthetic is approved for use by the U.S. Food & Drug Administration. Exemplary anesthetics may include, but are not limited to bupivacaine, tetracaine, procaine, proparacaine, propoxycaine, dimethocaine, cyclomethycaine, chloroprocaine, benzocaine, lidocaine, prilocain, levobupivicaine, ropivacaine, dibucaine, articaine, carticaine, etidocaine, mepivacaine, piperocaine, and trimecaine. In certain embodiments, the anesthetic is bupivacaine. In certain embodiments, the anesthetic agent is selected from the group consisting of bupivacaine, tetracaine, procaine, proparacaine, propoxycaine, dimethocaine, cyclomethycaine, chloroprocaine, benzocaine, lidocaine, prilocaine, levobupivacaine, ropivacaine, dibucaine, articaine, carticaine, etidocaine, mepivacaine, piperocaine, and trimecaine.

In certain embodiments, the therapeutic agent is an anesthetic agent. In certain embodiments, the therapeutic agent is a local anesthetic. In certain embodiments, the therapeutic agent is a sodium channel blocker anesthetic agent. In certain embodiments, the therapeutic agent is a site 1 sodium channel blocker anesthetic agent. In certain embodiments, the therapeutic agent is a potent site 1 sodium channel blocker anesthetic agent. In certain embodiments, the sodium channel blocker anesthetic agent is tetrodotoxin. In certain embodiments, the sodium channel blocker anesthetic agent is a saxitoxin (e.g., a member of the saxitocins class, an analog of saxitoxin). In certain embodiments, the sodium channel blocker anesthetic agent is saxitoxin. In certain embodiments, the sodium channel blocker anesthetic agent is neosaxitoxin. In certain embodiments, the sodium channel blocker anesthetic agent is gonyautoxin. In certain embodiments, the sodium channel blocker anesthetic agent is conotoxin (e.g., μ-conotoxin). In certain embodiments, the sodium channel blocker anesthetic agent is tetrodotoxin, saxitoxin, or conotoxin. In certain embodiments, the sodium channel blocker anesthetic agent is tetrodotoxin, saxitoxin, or neosaxitoxin. In certain embodiments, the therapeutic agents include bupivacaine and a sodium channel blocker anesthetic agent. In certain embodiments, the therapeutic agents include bupivacaine and a sodium channel blocker anesthetic agent that is tetrodotoxin. In certain embodiments, the therapeutic agent is a combination of anesthetic agents and does not comprise an antibiotic. In certain embodiments, the therapeutic agents include bupivacaine and a sodium channel blocker anesthetic agent that is tetrodotoxin and does not comprise ciprofloxacin. In certain embodiments, the first therapeutic agent is a local anesthetic. In certain embodiments, the first therapeutic agent is an amino-amide local anesthetic (e.g., bupivacaine, lidocaine, mepivacaine, etidocaine). In certain embodiments, the first therapeutic agent is an amino-ester local anesthetic (e.g., tetracaine, prilocaine, procaine, chloroprocaine, benzocaine).

In certain embodiments, the composition comprises between about 0.01-0.50% wt/vol of a second therapeutic agent that is a local anesthetic. In certain embodiments, the composition comprises between about 0.01-0.50% wt/vol of a therapeutic agent that is a sodium channel blocker anesthetic agent. In certain embodiments, the composition comprises between about 0.01-0.50% wt/vol of a therapeutic agent that is a site 1 sodium channel blocker anesthetic agent. In certain embodiments, the composition comprises between about 0.01-0.50% wt/vol of a therapeutic agent that is a sodium channel blocker anesthetic agent of tetrodotoxin. In certain embodiments, the composition comprises between about 0.01-0.50% wt/vol of a therapeutic agent that is a site 1 sodium channel blocker. In certain embodiments, the composition comprises between about 0.2-0.50% wt/vol of a therapeutic agent that is a sodium channel blocker anesthetic agent of tetrodotoxin. In certain embodiments, the composition comprises between about 0.1-0.50% wt/vol of a therapeutic agent that is a sodium channel blocker anesthetic agent of tetrodotoxin. In certain embodiments, the composition comprises between about 0.01-0.50% wt/vol, between about 0.03-0.50% wt/vol, between about 0.03-0.30% wt/vol, between about 0.1-0.50% wt/vol, between about 0.2-0.50% wt/vol, between about 0.1-0.45% wt/vol, between about 0.2-0.45% wt/vol, between about 0.25-0.50% wt/vol, between about 0.25-0.45% wt/vol, or between about 0.25-0.45% wt/vol, of a therapeutic agent that is a sodium channel blocker anesthetic agent. In certain embodiments, the composition comprises between about 0.01-0.50% wt/vol, between about 0.03-0.50% wt/vol, between about 0.03-0.30% wt/vol, between about 0.1-0.50% wt/vol, between about 0.2-0.50% wt/vol, between about 0.1-0.45% wt/vol, between about 0.2-0.45% wt/vol, between about 0.25-0.50% wt/vol, between about 0.25-0.45% wt/vol, or between about 0.25-0.45% wt/vol, of a therapeutic agent that is a site 1 sodium channel blocker anesthetic agent. In certain embodiments, the composition comprises between about 0.01-0.50% wt/vol, between about 0.03-0.50% wt/vol, between about 0.03-0.30% wt/vol, between about 0.2-0.50% wt/vol, between about 0.25-0.50% wt/vol, between about 0.25-0.45% wt/vol, or between about 0.25-0.45% wt/vol, of a therapeutic agent that is a sodium channel blocker anesthetic agent of tetrodotoxin. In certain embodiments, the composition comprises between about 0.03-0.30% wt/vol of a therapeutic agent that is a sodium channel blocker anesthetic agent. In certain embodiments, the composition comprises about 0.03% wt/vol of a sodium channel blocker anesthetic agent. In certain embodiments, the composition comprises about 0.3% wt/vol of a sodium channel blocker anesthetic agent. In certain embodiments, the composition comprises between about 0.03-0.30% wt/vol of a therapeutic agent that is a sodium channel blocker anesthetic agent of tetrodotoxin. In certain embodiments, the composition comprises about 0.03% wt/vol of tetrodotoxin. In certain embodiments, the composition comprises about 0.3% wt/vol of tetrodotoxin.

In certain embodiments, the therapeutic agent is an anti-inflammatory agent. The anti-inflammatory agent may be a non-steroidal anti-inflammatory agent or a steroidal anti-inflammatory agent. In certain embodiments, the therapeutic agent is a steroidal anti-inflammatory agent. In certain embodiments, the therapeutic agent is a steroid. Exemplary anti-inflammatory agents may include, but are not limited to, acetylsalicylic acid, amoxiprin, benorylate/benorilate, choline magnesium salicylate, diflunisal, ethenzamide, faislamine, methyl salicylate, magnesium salicylate, salicyl salicylate, salicylamide, diclofenac, aceclofenac, acemetacin, alclofenac, bromfenac, etodolac, indometacin, nabumetone, oxametacin, proglumetacin, sulindac, tolmetin, ibuprofen, alminoprofen, benoxaprofen, carprofen, dexibuprofen, dexketoprofen, fenbufen, fenoprofen, flunoxaprofen, flurbiprofen, ibuproxam, indoprofen, ketoprofen, ketorolac, loxoprofen, naproxen, oxaprozin, pirprofen, suprofen, tiaprofenic acid, mefenamic acid, flufenamic acid, meclofenamic acid, tolfenamic acid, phenylbutazone, ampyrone, azapropazone, clofezone, kebuzone, metamizole, mofebutazone, oxyphenbutazone, phenazone, phenylbutazone, sulfinpyrazone, piroxicam, droxicam, lornoxicam, meloxicam, tenoxicam, hydrocortisone, cortisone acetate, prednisone, prednisolone, methylprednisolone, dexamethasone, betamethasone, triamcinolone, beclometasone, fludrocortisone acetate, deoxycorticosterone acetate, and aldosterone. In certain embodiments, the anti-inflammatory agent is selected from the group consisting of acetylsalicylic acid, amoxiprin, benorylate/benorilate, choline magnesium salicylate, diflunisal, ethenzamide, faislamine, methyl salicylate, magnesium salicylate, salicyl salicylate, salicylamide, diclofenac, aceclofenac, acemetacin, alclofenac, bromfenac, etodolac, indometacin, nabumetone, oxametacin, proglumetacin, sulindac, tolmetin, ibuprofen, alminoprofen, benoxaprofen, carprofen, dexibuprofen, dexketoprofen, fenbufen, fenoprofen, flunoxaprofen, flurbiprofen, ibuproxam, indoprofen, ketoprofen, ketorolac, loxoprofen, naproxen, oxaprozin, pirprofen, suprofen, tiaprofenic acid, mefenamic acid, flufenamic acid, meclofenamic acid, tolfenamic acid, phenylbutazone, ampyrone, azapropazone, clofezone, kebuzone, metamizole, mofebutazone, oxyphenbutazone, phenazone, phenylbutazone, sulfinpyrazone, piroxicam, droxicam, lornoxicam, meloxicam, tenoxicam, hydrocortisone, cortisone acetate, prednisone, prednisolone, methylprednisolone, dexamethasone, betamethasone, triamcinolone, beclometasone, fludrocortisone acetate, deoxycorticosterone acetate, and aldosterone.

In various embodiments, combinations of various permeation enhancers and therapeutic agents have been observed to have a synergistic and heightened efficacy. In various aspects, such combinations may include, but are not limited to, ciprofloxacin and limonene. In various aspects, such combinations may include, but are not limited to, ciprofloxacin and sodium dodecyl sulfate. In various aspects such combinations may include, but are not limited to, sodium dodecyl sulfate, limonene, bupivacaine, and ciprofloxacin. In various aspects, such combination may include, but are not limited to, sodium dodecyl sulfate, limonene and ciprofloxacin.

In another aspect, provided herein are pharmaceutical compositions comprising at least one of the compositions as described herein, and optionally a pharmaceutically acceptable excipient. In certain embodiments, the pharmaceutical composition includes a combination of therapeutic agents. In certain embodiments, the pharmaceutical composition includes an antibiotic and an additional therapeutic agent. In certain embodiments, the pharmaceutical composition includes an antibiotic agent and an anti-inflammatory agent. In other embodiments, the pharmaceutical composition includes an antibiotic agent and an anesthetic agent. In certain embodiments, the pharmaceutical composition includes more than one antibiotic agent. In certain embodiments, the pharmaceutical composition comprises a therapeutically effective amount of the composition for use in treating a disease in a subject in need thereof.

In certain embodiments, the additional therapeutic agent is an anti-inflammatory agent (e.g., a steroid). In certain embodiments, the first therapeutic agent is an antibiotic and the additional therapeutic agent is an anti-inflammatory agent. In certain embodiments, the first therapeutic agent is an antibiotic and the additional therapeutic agent is a steroid. Steroids include, but are not limited to, cortisol, hydrocortisone acetate, cortisone acetate, tixocortol pivalate, prednisolone, methylprednisolone, prednisone, triamcinolone acetonide, triamcinolone alcohol, mometasone, amcinonide, budesonide, desonide, fluocinonide, fluocinolone acetonide, halcinonide, betamethasone, betamethasone sodium phosphate, dexamethasone, dexamethasone sodium phosphate, fluocortolone, hydrocortisone-17-valerate, halometasone, alclometasone dipropionate, betamethasone valerate, betamethasone dipropionate, prednicarbate, clobetasone-17-butyrate, clobetasol-17-propionate, fluocortolone caproate, fluocortolone pivalate, fluprednidene acetate, hydrocortisone-17-butyrate, hydrocortisone-17-aceponate, hydrocortisone-17-buteprate, ciclesonide, and prednicarbate. In some embodiments, the additional anti-inflammatory agent is dexamethasone.

In certain embodiments, the additional therapeutic agent is a β-lactamase inhibitor. In certain embodiments, the first therapeutic agent is an antibiotic (e.g., a β-lactam) and the additional therapeutic agent is a β-lactamase inhibitor. β-Lactamase inhibitors include, but are not limited to, avibactam, clavulanic acid, tazobactam, and sulbactam. The β-lactamase inhibitor may be particularly useful in compositions comprising a β-lactam antibiotic. The (3-lactamase inhibitor may increase the efficacy of a β-lactam antibiotic or allow for the (3-lactam antibiotic to be present in the composition in a lower concentration than for compositions not containing a β-lactamase inhibitor.

In certain embodiments, the additional therapeutic agent is an anesthetic agent. In certain embodiments, the additional therapeutic agent is bupivacaine.

Furthermore, after formulation with an appropriate pharmaceutically acceptable carrier in a desired dosage, the pharmaceutical compositions can be administered to humans and other animals.

Dosage forms include, but are not limited to, pharmaceutically acceptable emulsions, microemulsions, solutions, suspensions, syrups, and elixirs. In addition to the active compounds, the liquid dosage forms may contain inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethylformamide, oils (in particular, cottonseed, groundnut, corn, germ, olive, castor, and sesame oils), glycerol, tetrahydrofurfuryl alcohol, polyethylene glycols and fatty acid esters of sorbitan, and mixtures thereof. Besides inert diluents, the compositions can also include adjuvants such as wetting agents, emulsifying and suspending agents, and perfuming agents. In certain embodiments, the composition comprises a solubilizing agents such an Cremophor, alcohols, oils, modified oils, glycols, polysorbates, cyclodextrins, polymers, and combinations thereof.

It will also be appreciated that the compositions described herein can be employed in combination therapies, that is, the compounds and pharmaceutical compositions can be administered concurrently with, prior to, or subsequent to, one or more other desired therapeutics or medical procedures. The particular combination of therapies (therapeutics or procedures) to employ in a combination regimen will take into account compatibility of the desired therapeutics and/or procedures and the desired therapeutic effect to be achieved. It will also be appreciated that the therapies employed may achieve a desired effect for the same disorder (for example, a compound or composition disclosed herein may be administered concurrently with another anticancer agent), or they may achieve different effects (e.g., control of any adverse effects).

In certain embodiments, the composition comprises a diagnostic agent. In some embodiments, the diagnostic agent is an X-ray contrast agent. In some embodiments, the diagnostic agent comprises a radioactive isotope. In some embodiments, the diagnostic agent is a dye.

### Other Additives

In certain embodiments, the composition comprises one or more additional additives. For example, an additional additive may be a diluent, binding agent, preservative, buffering agent, lubricating agent, perfuming agent, antiseptic agent, or oil.

Exemplary diluents include calcium carbonate, sodium carbonate, calcium phosphate, dicalcium phosphate, calcium sulfate, calcium hydrogen phosphate, sodium phosphate lactose, sucrose, cellulose, microcrystalline cellulose, kaolin, mannitol, sorbitol, inositol, sodium chloride, dry starch, cornstarch, powdered sugar, and mixtures thereof.

Exemplary binding agents include starch (e.g., cornstarch and starch paste), gelatin, sugars (e.g., sucrose, glucose, dextrose, dextrin, molasses, lactose, lactitol, mannitol, etc.), natural and synthetic gums (e.g., acacia, sodium alginate, extract of Irish moss, panwar gum, ghatti gum, mucilage of isapol husks, carboxymethylcellulose, methylcellulose, ethylcellulose, hydroxyethylcellulose, hydroxypropyl cellulose, hydroxypropyl methylcellulose, microcrystalline cellulose, cellulose acetate, poly(vinyl-pyrrolidone), magnesium aluminum silicate (Veegum®), and larch arabogalactan), alginates, polyethylene oxide, polyethylene glycol, inorganic calcium salts, silicic acid, polymethacrylates, waxes, water, alcohol, and/or mixtures thereof.

Exemplary preservatives include antioxidants, chelating agents, antimicrobial preservatives, antifungal preservatives, antiprotozoan preservatives, alcohol preservatives, acidic preservatives, and other preservatives. In certain embodiments, the preservative is an antioxidant. In other embodiments, the preservative is a chelating agent. In certain embodiments, the preservative is benzalkonium chloride.

Exemplary antioxidants include alpha tocopherol, ascorbic acid, acorbyl palmitate, butylated hydroxyanisole, butylated hydroxytoluene, monothioglycerol, potassium metabisulfite, propionic acid, propyl gallate, sodium ascorbate, sodium bisulfite, sodium metabisulfite, and sodium sulfite.

Exemplary antifungal preservatives include butyl paraben, methyl paraben, ethyl paraben, propyl paraben, benzoic acid, hydroxybenzoic acid, potassium benzoate, potassium sorbate, sodium benzoate, sodium propionate, and sorbic acid.

Exemplary alcohol preservatives include ethanol, polyethylene glycol, phenol, phenolic compounds, bisphenol, chlorobutanol, hydroxybenzoate, and phenylethyl alcohol.

Exemplary acidic preservatives include vitamin A, vitamin C, vitamin E, beta-carotene, citric acid, acetic acid, dehydroacetic acid, ascorbic acid, sorbic acid, and phytic acid.

Other preservatives include tocopherol, tocopherol acetate, deteroxime mesylate, cetrimide, butylated hydroxyanisol (BHA), butylated hydroxytoluened (BHT), ethylenediamine, sodium lauryl sulfate (SLS), sodium lauryl ether sulfate (SLES), sodium bisulfite, sodium metabisulfite, potassium sulfite, potassium metabisulfite, Glydant® Plus, Phenonip®, methylparaben, Germall® 115, Germaben® II, Neolone®, Kathon®, and Euxyl®.

Exemplary buffering agents include citrate buffer solutions, acetate buffer solutions, phosphate buffer solutions, ammonium chloride, calcium carbonate, calcium chloride, calcium citrate, calcium glubionate, calcium gluceptate, calcium gluconate, D-gluconic acid, calcium glycerophosphate, calcium lactate, propanoic acid, calcium levulinate, pentanoic acid, dibasic calcium phosphate, phosphoric acid, tribasic calcium phosphate, calcium hydroxide phosphate, potassium acetate, potassium chloride, potassium gluconate, potassium mixtures, dibasic potassium phosphate, monobasic potassium phosphate, potassium phosphate mixtures, sodium acetate, sodium bicarbonate, sodium chloride, sodium citrate, sodium lactate, dibasic sodium phosphate, monobasic sodium phosphate, sodium phosphate mixtures, tromethamine, magnesium hydroxide, aluminum hydroxide, alginic acid, pyrogen-free water, isotonic saline, Ringer's solution, ethyl alcohol, and mixtures thereof.

Exemplary lubricating agents include magnesium stearate, calcium stearate, stearic acid, silica, talc, malt, glyceryl behanate, hydrogenated vegetable oils, polyethylene glycol, sodium benzoate, sodium acetate, sodium chloride, leucine, magnesium lauryl sulfate, sodium lauryl sulfate, and mixtures thereof.

Exemplary natural oils include almond, apricot kernel, avocado, babassu, bergamot, black current seed, borage, cade, camomile, canola, caraway, carnauba, castor, cinnamon, cocoa butter, coconut, cod liver, coffee, corn, cotton seed, emu, eucalyptus, evening primrose, fish, flaxseed, geraniol, gourd, grape seed, hazel nut, hyssop, isopropyl myristate, jojoba, kukui nut, lavandin, lavender, lemon, Litsea cubeba, macademia nut, mallow, mango seed, meadowfoam seed, mink, nutmeg, olive, orange, orange roughy, palm, palm kernel, peach kernel, peanut, poppy seed, pumpkin seed, rapeseed, rice bran, rosemary, safflower, sandalwood, sasquana, savoury, sea buckthorn, sesame, shea butter, silicone, soybean, sunflower, tea tree, thistle, tsubaki, vetiver, walnut, and wheat germ oils. Exemplary synthetic oils include, but are not limited to, butyl stearate, caprylic triglyceride, capric triglyceride, cyclomethicone, diethyl sebacate, dimethicone 360, isopropyl myristate, mineral oil, octyldodecanol, oleyl alcohol, silicone oil, and mixtures thereof.

In addition to the active ingredients, the liquid dosage forms may comprise inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethylformamide, oils (e.g., cottonseed, groundnut, corn, germ, olive, castor, and sesame oils), glycerol, tetrahydrofurfuryl alcohol, polyethylene glycols and fatty acid esters of sorbitan, and mixtures thereof.

The composition may comprise water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethylformamide, oils (e.g., cottonseed, groundnut, corn, germ, olive, castor, and sesame oils), glycerol, tetrahydrofurfuryl alcohol, polyethylene glycols and fatty acid esters of sorbitan, and mixtures thereof.

Formulations suitable for administration (e.g., to the ear canal) include, but are not limited to, liquid and/or semi-liquid preparations such as liniments, lotions, oil-in-water, and/or water-in-oil emulsions such as creams, ointments, and/or pastes, and/or solutions and/or suspensions. Topically administrable formulations may, for example, comprise from about 1% to about 10% (w/w) therapeutic agent, although the concentration of the therapeutic agent can be as high as the solubility limit of the active ingredient in the solvent.

### Methods of Treatment and Uses

Provided herein are methods of the compositions described herein for treating a disease or condition in a subject in need thereof. In certain embodiments, the compositions described herein are used in treating (e.g., sustained treating of) pain. In certain embodiments, the compositions described herein are used in treating pain associated with an infectious disease (e.g., sustained pain treatment). In certain embodiments, the compositions described herein are used in treating pain (e.g., sustained pain treatment) associated with an ear disease or a bacterial infection. In certain embodiments, the compositions described herein are used in sustained pain treatment. In certain embodiments, the compositions described herein are used in sustained pain treatment for pain associated with an infectious disease, an ear disease, or a bacterial infection.

Methods of using the various embodiments of the compositions described herein are generally directed to methods of treating an infectious disease, an ear disease, and/or a condition (e.g., treating pain, sustained pain treatment) associated with an infectious disease and/or an ear disease. In certain embodiments, the compositions described herein are used in a method of treating pain. In certain embodiments, the compositions described herein are used in a method of treating an infectious disease. In certain embodiments, the matrix forming agents described herein are used in a method of treating an infectious disease. In certain embodiments, the compositions described herein are used in a method of treating an ear disease. In certain embodiments, the compositions described herein are used in a method of treating an infectious ear disease. Methods of using the various embodiments of the compositions described herein are generally directed to methods of treating an infectious disease. In various aspects, the compositions may be used to deliver therapeutic or diagnostic agents across the tympanic membrane. Therefore, the compositions are particularly useful in treating diseases and/or conditions of the middle and/or inner ear. In certain embodiments, the compositions described herein are used in a method of treating diseases and/or conditions of the middle ear. In certain embodiments, the compositions described herein are used in a method of treating diseases and/or conditions of the inner ear.

In certain embodiments, the subject described herein is a human. In certain embodiments, the subject is a non-human animal. In certain embodiments, the subject is a mammal. In certain embodiments, the subject is a non-human mammal. In certain embodiments, the subject is a domesticated animal, such as a dog, cat, cow, pig, horse, sheep, or goat. In certain embodiments, the subject is a companion animal, such as a dog or cat. In certain embodiments, the subject is a livestock animal, such as a cow, pig, horse, sheep, or goat. In certain embodiments, the subject is a zoo animal. In another embodiment, the subject is a research animal, such as a rodent (e.g., mouse, rat), dog, pig, or non-human primate.

In various aspects, compositions described herein can be used to treat ear diseases, including, but not limited to, ear infections, development of fibroids in the middle ear, or otosclerosis. In certain embodiments, the matrix forming agents described herein can be used to treat ear diseases, including, but not limited to, ear infections, development of fibroids in the middle ear, or otosclerosis. In various other aspects, compositions described herein may be used may treat vertigo, Meniere's disease, mastoiditis, cholesteatoma, labyrinthitis, perilymph fistula, superior canal dehiscence syndrome, otorrhea, otalgia, tinnitus, barotrauma, cancers of the ear, autoimmune inner ear disease acoustic neuroma, benign paroxysmal positional vertigo, herpes zoster oticus, purulent labyrinthitis, vestibular neuronitis, eardrum perforation, or myringitis. In various other aspects, compositions described herein may be used may treat vertigo, Meniere's disease, mastoiditis, cholesteatoma, labyrinthitis, perilymph fistula, superior canal dehiscence syndrome, otorrhea, otalgia, tinnitus, barotrauma, cancers of the ear, autoimmune inner ear disease acoustic neuroma, benign paroxysmal positional vertigo, herpes zoster oticus, purulent labyrinthitis, vestibular neuronitis, eardrum perforation, or myringitis. In certain embodiments, the matrix forming agents described herein may be used may treat vertigo, Meniere's disease, mastoiditis, cholesteatoma, labyrinthitis, perilymph fistula, superior canal dehiscence syndrome, otorrhea, otalgia, tinnitus, barotrauma, cancers of the ear, autoimmune inner ear disease acoustic neuroma, benign paroxysmal positional vertigo, herpes zoster oticus, purulent labyrinthitis, vestibular neuronitis, eardrum perforation, or myringitis. In some embodiments, the methods disclosed herein are used for treating otitis media (OM). Different forms of OM, which may be treated by the methods disclosed herein, may be differentiated by the presence of fluid (effusion) and/or by the duration or persistence of inflammation. In certain embodiments, the infectious disease is acute otitis media, chronic otitis media, or secretory otitis media. Effusions, if present, can be of any consistency, from water-like (serous) to viscid and mucous-like (mucoid), to pus-like (purulent); duration is classified as acute, subacute, or chronic. OM with effusion (OME) indicates inflammation with middle ear fluid (MEF), but in the absence of any indications of acute infection. Acute OM (AOM), with or without effusion, is characterized by rapid onset of the signs and symptoms associated with acute infection in the middle ear (e.g., otalgia, fever). In some embodiments, the methods are used for treating otitis media associated with infection by any of a number of pathogenic bacteria, including, for example, Streptococcus pneumoniae, Haemophilus influenzae, and Moraxella catarrhalis.

The infectious disease may be a bacterial infection. In certain embodiments, the bacterial infection is a Streptococcus, Haemophilus, or Moraxella infection. In certain embodiments, the bacterial infection is a Staphylococcus, Escherichia, or Bacillus infection. In certain embodiments, the bacterial infection is an H. influenzae infection. In certain embodiments, the bacterial infection is a S. pneumoniae infection. In certain embodiments, the bacterial infection is an M. catarrhalis infection. In certain embodiments, the infectious disease is an ear infection. In certain embodiments, the infectious disease is otitis media.

In various embodiments, administration of the compositions described herein consists of applying the composition into a subject's ear canal. In certain embodiments, applying the composition into a subject's ear canal comprises spraying the composition into a subject's ear canal. In certain embodiments, administration of the compositions described herein consists of applying the composition into the inner ear of a subject. In certain embodiments, administration of the compositions described herein consists of applying the composition into the middle ear of a subject. In certain embodiments, administration of the compositions described herein consists of applying the composition into the inner ear, sinuses, the eye, vagina, or skin of a subject. In certain embodiments, administration of the compositions described herein consists of applying the composition into the sinuses of a subject. In certain embodiments, administration of the compositions described herein consists of applying the composition into the eye of a subject. In certain embodiments, administration of the compositions described herein consists of applying the composition into the vagina of a subject. In certain embodiments, administration of the compositions described herein consists of applying the composition to the skin of a subject. A subject for treatment can be any mammal in need of treatment. In various aspects, the composition is in direct contact with the tympanic membrane for about 1 day to about 30 days. In various aspects, the composition is in contact with the tympanic membrane from about 1 day to about 3 days, from about 3 days to about 7 days, from about 7 days to about 14 days, from about 14 days to about 21 days, or from about 21 days to about 30 days. In various embodiments, the composition forms a sustained release reservoir, in contact with the tympanic membrane. In various aspects, the composition is applied into the ear canal as a liquid, and the composition gels in situ on the surface of the tympanic membrane. When in contact with the tympanic membrane, the therapeutic agent penetrates the tympanic membrane and is delivered to the middle ear. In various embodiments, the delivery across the tympanic membrane is a sustained release of the therapeutic agent over a number of days. The numbers of days that the composition can be in contact with the tympanic membrane can be, but is not limited to, 5 days, 7 days, 10 days, 14 days, 21 days, or 30 days. The composition may be applied singly, or repeatedly in the course of treatment. In various aspects, the composition may be periodically administered from about every 1 day to about every 7 days, from about every 1 day to about every 14 days, or from about every 1 day to about every 30 days. In various embodiments, the composition is naturally extruded from the subject at the end of treatment via natural processes similar to extrusion of ear wax. In certain embodiments, the composition may naturally break down, and its degradation products may be eliminated by the subject. In various embodiments, administration of the compositions described herein comprises adding the matrix forming agent, the permeation enhancer, and the therapeutic agent to the ear canal; then adding a second therapeutic agent to the ear canal; and mixing the matrix forming agent, the permeation enhancer, and the therapeutic agent on the ear canal. In certain embodiments, the second therapeutic agent is an anesthetic. In certain embodiments, the second therapeutic agent is a local anesthetic.

In various embodiments, administration of the compositions described herein comprises adding the matrix forming agent to the ear canal; adding the permeation enhancer to the ear canal; adding the therapeutic agent to the ear canal; and mixing the matrix forming agent, the permeation enhancer, and the therapeutic agent on the ear canal. In various embodiments, administration of the compositions described herein comprises adding the matrix forming agent to the ear canal; adding the permeation enhancer to the ear canal; adding the therapeutic agent to the ear canal; adding an additional therapeutic agent to the ear canal; and mixing the matrix forming agent, the permeation enhancer, and the therapeutic agents on the ear canal. In certain embodiments, adding the therapeutic agent and adding the permeation enhancer to the ear canal comprises spraying the therapeutic agent and spraying the permeation enhancer into the ear canal.

In various embodiments, administration of the compositions described herein comprises adding the therapeutic agent to the ear canal; adding the permeation enhancer to the ear canal; adding the matrix forming agent to the ear canal; and mixing the matrix forming agent, the permeation enhancer, and the therapeutic agent on the ear canal. In various embodiments, administration of the compositions described herein comprises adding the therapeutic agent to the ear canal; adding an additional therapeutic agent to the ear canal; adding the permeation enhancer to the ear canal; adding the matrix forming agent to the ear canal; and mixing the matrix forming agent, the permeation enhancer, and the therapeutic agents on the ear canal. In certain embodiments, adding the therapeutic agent and adding the permeation enhancer to the ear canal comprises spraying the therapeutic agent and spraying the permeation enhancer into the ear canal. In certain embodiments, the therapeutic agent is an antibiotic or anesthetic agent. In certain embodiments, the therapeutic agent is an antibiotic. In certain embodiments, the therapeutic agent is an anesthetic agent. In certain embodiments, the permeation enhancer is bupivacaine.

In various embodiments, administration of the compositions described herein comprises adding a composition including one or more therapeutic agents, one or more permeation enhancers, and one or more matrix forming agents to the ear canal; and subsequently adding a composition comprising no therapeutic agents or one or more therapeutic agents, no permeation enhancers or one or more permeation enhancers, and no matrix forming agents or one or more matrix forming agents to the ear canal. In certain embodiments, the subsequent addition of the one or more therapeutic agents comprises therapeutic agents that are the same as in the first addition of the one or more therapeutic agents. In certain embodiments, the subsequent addition of the one or more therapeutic agents comprises therapeutic agents that are different from those in the first addition of the one or more therapeutic agents. In certain embodiments, the subsequent addition of permeation enhancers comprises permeation enhancers that are the same as in the first addition of the permeation enhancers. In certain embodiments, the subsequent addition of the permeation enhancers comprises permeation enhancers that are different from those in the first addition of the permeation enhancers. In certain embodiments, the subsequent addition of matrix forming agents comprises matrix forming agents that are the same as in the first addition of the matrix forming agents. In certain embodiments, the subsequent addition of the matrix forming agents comprises matrix forming agents that are different from those in the first addition of the matrix forming agents. In certain embodiments, the time interval between the adding of the first composition and second composition is about one minute. In certain embodiments, the time interval between the adding of the first composition and second composition is less than one minute. In certain embodiments, the time interval between the adding of the first composition and second composition is more than one minute.

In certain embodiments, a dose is determined based on the minimum inhibitory concentration needed at the site of infection. Without being bound to a particular theory, in various aspects the minimum inhibitory concentration for H. influenza or S. pneumoniae middle ear infections is about 4 μg/mL for ciprofloxacin. In various aspects, a typical dose will require approximately 12 μg of ciprofloxacin, based on an average middle ear volume of 3 mL. In various embodiments, the compositions will comprise sufficient dose to delivery 12 g of ciprofloxacin to the middle ear.

Without being bound to a particular theory, in various aspects the minimum dosage concentration required for treating pain associated with H. influenza or S. pneumoniae middle ear infections is about 0.36 μg/mL for bupivacaine and/or about 0.32 μg/mL for tetrodotoxin. In various aspects, the minimum dosage concentration achieved (e.g., on the middle ear side during a permeation experiment using dissected ear drum, or in the middle ear) for treating pain associated with H. influenza or S. pneumoniae middle ear infections is about 8 μg/mL (about 25 μM) for bupivacaine and/or about 0.3 ng/mL (about 1 nM) for tetrodotoxin.

In various aspects, the administration of the composition comprises a single application. In other aspects, the administration of the composition comprises multiple applications. For example, the composition may be administered two, three, four, or more times. In certain embodiments, the composition is administered repeatedly until the desired clinical outcome is achieved. For example, the infection is resolved. In certain embodiments, the administration of the composition comprises a first administration of the composition, followed by a second administration of the composition after a period of time. In certain embodiments, the period of time between the first administration of the composition and the second administration of the composition is a week. In certain embodiments, the period of time between the first administration of the composition and the second administration of the composition is more than one week. In certain embodiments, the period of time between the first administration of the composition and the second administration of the composition is one month. In certain embodiments, the period of time between the first administration of the composition and the second administration of the composition is more than one month. In various embodiments, administration of the compositions described herein comprises a first administration of a composition without a local anesthetic to the ear canal; followed by a second administration of a composition without a local anesthetic to the ear canal. In certain embodiments, administration of the compositions described herein comprises a first administration of a composition with a local anesthetic to the ear canal; followed by a second administration of a composition without a local anesthetic to the ear canal.

In various embodiments, administration of the compositions described herein comprises a first administration of a composition without a local anesthetic to the ear canal; followed by a second administration of a composition without a permeation enhancer other than a local anesthetic to the ear canal. In certain embodiments, administration of the compositions described herein comprises a first administration of a composition with a local anesthetic to the ear canal; followed by a second administration of a composition without a permeation enhancer other than local anesthetic to the ear canal. In certain embodiments, the composition administered first and the composition administered second are the same. In certain embodiments, the composition administered first and the composition administered second are different.

Provided herein are methods of delivering a composition of the disclosure to the surface of tympanic membrane of a subject. In certain embodiments, the subject has an ear disease. In some embodiments, the subject has otitis media. In some embodiments, the subject is a human. In certain embodiments, the subject is a domesticated animal, such as a dog, cat, cow, pig, horse, sheep, or goat.

In certain embodiments, the method of delivering comprises administering the composition into the ear canal via an applicator. In certain embodiments, the method of delivering comprises placing drops of the composition into the ear canal. In some embodiments, the drops are delivered from a dropper (e.g., pipet, eye dropper). In some embodiments, the drops are delivered by a syringe. The syringe may be attached to a needle, rigid catheter, or flexible catheter. In certain embodiments, the method of delivering comprises administering the composition on the round window membrane to deliver the composition to the inner ear.

In certain embodiments, the method of delivering comprises placing a dose of the composition into the ear canal using a catheter. In some embodiments the catheter is attached to a syringe. In some embodiments, the catheter is rigid. In some embodiments the catheter is flexible. In certain embodiments, the method of delivering comprises placing a dose of the composition into the ear canal using a needle. In some embodiments, the needle is attached to a syringe. In some embodiments, the needle has a blunt tip.

In certain embodiments, the method of delivering comprises placing a dose of the composition into the ear canal using a double barrel syringe. The double barrel syringe may be used to keep two components of a composition until mixing of the two components occurs during administration (e.g., in situ). In some embodiments, the double barrel syringe is attached to a single catheter or needle. In some embodiments, each barrel of the double barrel syringe is attached to a separate needle or catheter.

In certain embodiments, the method of treating an infectious disease or ear disease comprises instructing a subject to administer, or providing instructions to a subject for self-administration of, the composition.

In another aspect, provided herein are methods of eradicating a biofilm in a subject comprising administering to a subject in need thereof, a composition described herein to a subject in need thereof. In another aspect, provided herein are methods of eradicating a biofilm comprising contacting the biofilm with a composition described herein. In another aspect, provided herein are methods of inhibiting formation of a biofilm in a subject, comprising administering to a subject in need thereof a composition described herein to a subject in need thereof. In another aspect, provided herein are methods of inhibiting formation of a biofilm comprising contacting a surface with a composition described herein.

In another aspect, provided herein are uses of compositions described herein to treat and/or prevent a disease or condition (e.g., an infectious disease, ear disease, bacterial infection, pain) and/or a condition associated with the disease (e.g., pain associated with an infectious disease, ear disease, bacterial infection) in a subject in need thereof, the use comprising administering to the subject a therapeutically effective amount of compositions described herein. In certain embodiments, provided are uses of compositions described herein to treat pain, the use comprising administering to the subject a therapeutically effective amount of compositions described herein.

### Kits

Provided herein are kits comprising any of the compositions described herein, which may additionally comprise the compositions in sterile packaging. Provided herein are kits comprising any of the compositions or matrix-forming agents described herein, which may additionally comprise the compositions or matrix-forming agents in sterile packaging. The kits may comprise two containers for two-part, matrix-forming agents. The therapeutic agent may be included in one or both of the containers of the matrix forming agent, or the therapeutic agent may be packaged separately. The permeation enhancer may be included in one or both of the containers of the matrix forming agent, or the permeation enhancer may be packaged separately. In various aspects the kits may comprise a bottle or bottles, and a dropper or syringe for each bottle. In certain embodiments, the kits are used for treating a disease, condition (e.g., pain), and/or condition associated with a disease (e.g., pain associated with an ear disease, infectious disease, bacterial infection) described herein (e.g., an ear disease, infectious disease, bacterial infection).

In certain embodiments, the kit comprises one or more droppers (e.g., pipet, eye dropper). In certain embodiments, the kit comprises one or more syringe. In some embodiments, the syringe is pre-loaded with the composition, or one or more component of the composition. In certain embodiments, the kit comprises one or more needle (e.g., blunt-tipped needle). In certain embodiments, the kit comprises one or more catheter (e.g., flexible catheter).

In certain the kit comprises a double barrel syringe. In some embodiments, the double barrel syringe is pre-loaded with two components of the composition. In some embodiments, the double barrel syringe is attached to a single catheter or needle. In some embodiments, each barrel of the double barrel syringe is attached to a separate needle or catheter.

In certain embodiments, a kit described herein further includes instructions for using the kit, such as instructions for using the kit in a method of the disclosure (e.g., instructions for administering a compound or pharmaceutical composition described herein to a subject). A kit described herein may also include information as required by a regulatory agency such as the U.S. Food and Drug Administration (FDA).

### EXAMPLES

In order that the present disclosure may be more fully understood, the following examples are set forth. The synthetic and biological examples described in this application are offered to illustrate the compounds, pharmaceutical compositions, and methods provided herein and are not to be construed in any way as limiting their scope.

### Example 1. Rheology

The exemplary compositions were analyzed for favorable properties with regard to gelation and syringeability. The rheology data, including the storage modulus (G′) and the loss modulus (G″), were plotted over a temperature range of the composition. Trans-tympanic and biocompatibility experiments are also performed.

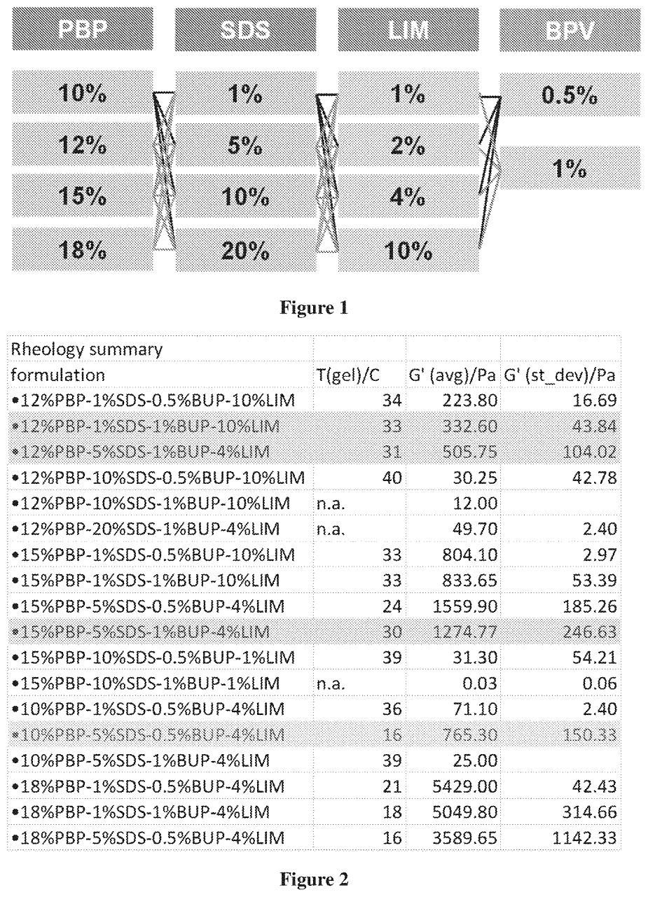

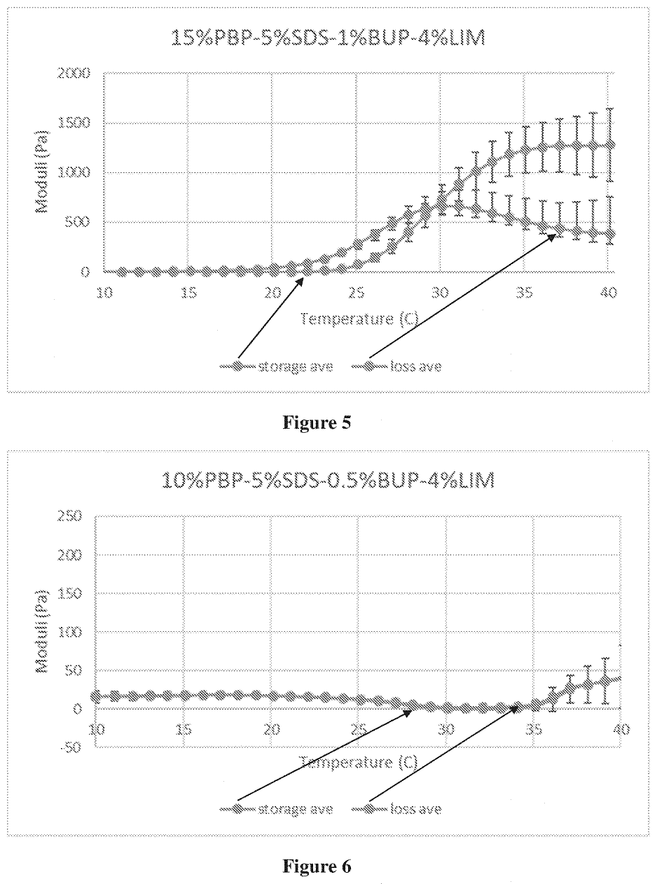

Exemplary viable compositions with reasonable gelation and syringeability properties include compositions of: 12% PBP-1% SDS-0.5% BUP-10% LIM, 12% PBP-1% SDS-1% BUP-10% LIM, 12% PBP-5% SDS-1% BUP-4% LIM, 12% PBP-10% SDS-0.5% BUP-10% LIM, 12% PBP-10% SDS-1% BUP-10% LIM, 12% PBP-20% SDS-1% BUP-4% LIM, 15% PBP-1% SDS-0.5% BUP-10% LIM, 15% PBP-1% SDS-1% BUP-10% LIM, 15% PBP-5% SDS-0.5% BUP-4% LIM, 15% PBP-5% SDS-1% BUP-4% LIM, 15% PBP-10% SDS-0.5% BUP-1% LIM, 15% PBP-10% SDS-1% BUP-1% LIM, 10% PBP-1% SDS-0.5% BUP-4% LIM, 10% PBP-5% SDS-0.5% BUP-4% LIM, 10% PBP-5% SDS-1% BUP-4% LIM, 18% PBP-1% SDS-0.5% BUP-4% LIM, 18% PBP-1% SDS-1% BUP-4% LIM, and 18% PBP-5% SDS-0.5% BUP-4% LIM. Each of the compositions are provided as percentage weight/vol. See FIGS. 1-6.

### Example 2. Formulations and Properties with Reference to Gelation, Syringeability, Storage Modulus, and Gelation Temperature

TABLE 1
Data summary for composition formulation optimization, group 1.

| | | | Gelation | Gelation | | | |
|---|---|---|---|---|---|---|---|
| | | | Test: | Test: | | Storage | |
| | | | Liquid | Turns | | modulus | Gelation |
| | | Solution | under room | Solid at body | Syringeability | at 37° C. | Temp |
| Group-1 | | Tested | temp.? | temp.? | Test:^{X} | (Pa) | (° C.) |
| Sub- | sub-sub- | 12%, 1%, | Yes | Most | 1 | | |
| group 1-1 | group 1-1-1 | 0.5%, 1% | | | | | |
| | | 12%, 1%, | Yes | Most | 1 | | |
| | | 0.5%, 2% | | | | | |
| | | 12%, 1%, | Yes | Some | 1 | | |
| | | 0.5%, 4% | | | | | |
| | | 12%, 1%, | Yes | Most | 1 | 223.8 ± 16.7 | 34 |
| | | 0.5%, 10% | | | | | |
| | sub-sub- | 12%, 1%, | Yes | Yes | 1 | | |
| | group 1-1-2 | 1%, 1% | | | | | |
| | | 12%, 1%, | Yes | Yes | 1 | | |
| | | 1%, 2% | | | | | |
| | | 12%, 1%, | Yes | Some | 1 | | |
| | | 1%, 4% | | | | | |
| | | 12%, 1%, | Yes | Some | 1 | 332.6 ± 43.8 | 33 |
| | | 1%, 10% | | | | | |
| Sub- | sub-sub- | 12%, 5%, | Yes | Yes | 4 | | |
| group 1-2 | group 1-2-1 | 0.5%, 1% | | | | | |
| | | 12%, 5%, | Yes | Yes | 2 | | |
| | | 0.5%, 2% | | | | | |
| | | 12%, 5%, | Yes | Yes | 3 | | |
| | | 0.5%, 4% | | | | | |
| | | 12%, 5%, | Yes | Yes | 4 | | |
| | | 0.5%, 10% | | | | | |
| | sub-sub- | 12%, 5%, | Yes | Yes | 3 | | |
| | group 1-2-2 | 1%, 1% | | | | | |
| | | 12%, 5%, | Yes | Yes | 2 | | |
| | | 1%, 2% | | | | | |
| | | 12%, 5%, | Yes | Yes | 2 | 505.8 ± 104.2 | 31 |
| | 1%, 4% | | | | | | |
| | | 12%, 5%, | Yes | Yes | 3 | | |
| | 1%, 10% | | | | | | |
| Sub- | sub-sub- | 12%, 10%, | Yes | No, for 10 s, | 2 | | |
| group 1-3 | group 1-3-1 | 0.5%, 1% | | 20 s, 30 s, 40 s | | | |
| | | 12%, 10%, | No | Some | 3 | | |
| | | 0.5%, 2% | | | | | |
| | | 12%, 10%, | Yes | No | 3 | | |
| | | 0.5%, 4% | | | | | |
| | | 12%, 10%, | Yes | Some | 3 | 30.3 ± 42.8 | 40 |
| | | 0.5%, 10% | | | | | |
| | sub-sub- | 12%, 10%, | Yes, but | No, for 10 s, | 2 | | |
| | group 1-3-2 | 1%, 1% | viscous. Got | 20 s, 30 s, 40 s | | | |
| | | | less viscous | | | | |
| | | | over time | | | | |
| | | 12%, 10%, | Yes | Yes | 4 | | |
| | | 1%, 2% | | | | | |
| | | 12%, 10%, | Yes | Yes | 4 | | |
| | | 1%, 4% | | | | | |
| | | 12%, 10%, | Yes | Yes | 4 | 12 | n.a. |
| | | 1%, 10% | | | | | |
| Sub- | sub-sub- | 12%, 20%, | Yes | No, for 10 s, | 2 | | |
| group 1-4 | group 1-4-1 | 0.5%, 1% | | 20 s, 30 s, 40 s | | | |
| | | 12%, 20%, | Yes | No | 4 | | |
| | | 0.5%, 2% | | | | | |
| | | 12%, 20%, | No | No | 4 | | |
| | | 0.5%, 4% | | | | | |
| | | 12%, 20%, | No | No | 4 | | |
| | | 0.5%, 10% | | | | | |
| | sub-sub- | 12%, 20%, | No, but got | Yes, for 10 s, | 1 | | |
| | group 1-4-2 | 1%, 1% | liquid over | partially | | | |
| | | | time | melted for | | | |
| | | | | longer | | | |
| | | 12%, 20%, | No | No | 4 | | |
| | | 1%, 2% | | | | | |
| | | 12%, 20%, | Mostly | Yes | 3 | 49.7 | n.a. |
| | | 1%, 4% | | | | | |
| | | 12%, 20%, | Yes | No | 4 | | |
| | | 1%, 10% | | | | | |

^{X}syringeability test results range from 1 to 5, where 1 is good syringeability (e.g., can be syringeable as liquid through a soft catheter without clogging) and 5 is poor syringeability (e.g., low ability to be syringeable as liquid through a soft catheter without clogging)

TABLE 2
Data summary for exemplary composition formulation optimization, group 2.

| | | | Gelation | Gelation | | | |
|---|---|---|---|---|---|---|---|
| | | | Test: | Test: | | Storage | |
| | | | Liquid | Turns | | modulus | Gelation |
| | | Solution | under room | Solid at body | Syringeability | at 37° C. | Temp |
| Group-2 | | Tested | temp.? | temp.? | Test:^{X} | (Pa) | (° C.) |
| Sub- | sub-sub- | 15%, 1%, | Yes | Some | 1 | | |
| group 2-1 | group 2-1-1 | 0.5%, 1% | | | | | |
| | | 15%, 1%, | Yes | Most | 1 | | |
| | | 0.5%, 2% | | | | | |
| | | 15%, 1%, | Yes | Most | 1 | | |
| | | 0.5%, 4% | | | | | |
| | | 15%, 1%, | Yes | Some | 1 | 804.1 ± 2.97 | 33 |
| | | 0.5%, 10% | | | | | |
| | sub-sub- | 15%, 1%, | Yes | Mostly-Yes | 1 | | |
| | group 2-1-2 | 1%, 1% | | | | | |
| | | 15%, 1%, | Yes | Yes | 1 | | |
| | | 1%, 2% | | | | | |
| | | 15%, 1%, | Yes | Yes | 1 | | |
| | | 1%, 4% | | | | | |
| | | 15%, 1%, | Yes | Some | 1 | 833.7 ± 53.4 | 33 |
| | | 1%, 10% | | | | | |
| Sub- | sub-sub- | 15%, 5%, | Yes | Yes | 3 | | |
| group 2-2 | group 2-2-1 | 0.5%, 1% | | | | | |
| | | 15%, 5%, | Yes | Yes | 3 | | |
| | | 0.5%, 2% | | | | | |
| | | 15%, 5%, | Yes | Yes | 2 | 1559.9 ± 185.3 | 24 |
| | | 0.5%, 4% | | | | | |
| | | 15%, 5%, | Yes | Yes | 3 | | |
| | | 0.5%, 10% | | | | | |
| | sub-sub- | 15%, 5%, | Yes | Yes | 3 | | |
| | group 2-2-2 | 1%, 1% | | | | | |
| | | 15%, 5%, | Yes | Yes | 2 | | |
| | | 1%, 2% | | | | | |
| | | 15%, 5%, | Yes | Yes | 2 | 1274.8 ± 246.6 | 30 |
| | | 1%, 4% | | | | | |
| | | 15%, 5%, | Yes | Yes | 3 | | |
| | | 1%, 10% | | | | | |
| Sub- | sub-sub- | 15%, 10%, | Slightly | Yes | 2 | 31.3 ± 54.2 | 39 |
| group 2-3 | group 2-3-1 | 0.5%, 1% | | | | | |
| | | 15%, 10%, | No | Yes | 4 | | |
| | | 0.5%, 2% | | | | | |
| | | 15%, 10%, | No | Yes | 4 | | |
| | | 0.5%, 4% | | | | | |
| | | 15%, 10%, | No | Yes | 4 | | |
| | | 0.5%, 10% | | | | | |
| | sub-sub- | 15%, 10%, | Slightly | Yes | 2 | 0.03 ± 0.06 | n.a. |
| | group 2-3-2 | 1%, 1% | | | | | |
| | | 15%, 10%, | No | No | 2 | | |
| | | 1%, 2% | | | | | |
| | | 15%, 10%, | Slightly | Yes | 4 | | |
| | | 1%, 4% | | | | | |
| | | 15%, 10%, | No | Yes | 4 | | |
| | | 1%, 10% | | | | | |
| Sub- | sub-sub- | 15%, 20%, | No | No | 3 | | |
| group 2-4 | group 2-4-1 | 0.5%, 1% | | | | | |
| | | 15%, 20%, | No | No | 2 | | |
| | | 0.5%, 2% | | | | | |
| | | 15%, 20%, | No | Yes | 4 | | |
| | | 0.5%, 4% | | | | | |
| | | 15%, 20%, | No | Yes | 4 | | |
| | | 0.5%, 10% | | | | | |
| | sub-sub- | 15%, 20%, | No | No | 3 | | |
| | group 2-4-2 | 1%, 1% | | | | | |
| | | 15%, 20%, | No | Some (very | 3 | | |
| | | 1%, 2% | | viscous liquid) | | | |
| | | 15%, 20%, | No | Yes | 4 | | |
| | | 1%, 4% | | | | | |
| | | 15%, 20%, | No | Yes | 4 | | |
| | | 1%, 10% | | | | | |

^{X}syringeability test results range from 1 to 5, where 1 is good syringeability (e.g., can be syringeable as liquid through a soft catheter without clogging) and 5 is poor syringeability (e.g., low ability to be syringeable as liquid through a soft catheter without clogging)

TABLE 3
Data summary for exemplary composition formulation optimization, group 3.

| | | | Gelation | Gelation | | | |
|---|---|---|---|---|---|---|---|
| | | | Test: | Test: | | Storage | |
| | | | Liquid | Turns | | modulus | Gelation |
| | | Solution | under room | Solid at body | Syringeability | at 37° C. | Temp |
| Group-3 | | Tested | temp.? | temp.? | Test:^{X} | (Pa) | (° C.) |
| Sub- | sub-sub- | 10%, 1%, | Yes | Most | 1 | | |
| group 3-1 | group 3-1-1 | 0.5%, 1% | | | | | |
| | | 10%, 1%, | Yes | Some | 1 | | |
| | | 0.5%, 2% | | | | | |
| | | 10%, 1%, | Yes | Yes | 1 | | |
| | | 0.5%, 3% | | | | | |
| | | 10%, 1%, | Yes | Yes | 1 | 71.1 ± 2.4 | 36 |
| | | 0.5%, 4% | | | | | |
| | sub-sub- | 10%, 1%, | Yes | Some | 1 | | |
| | group 3-1-2 | 1%, 1% | | | | | |
| | | 10%, 1%, | Yes | No | 1 | | |
| | | 1%, 2% | | | | | |
| | | 10%, 1%, | Yes | No | 1 | | |
| | | 1%, 3% | | | | | |
| | | 10%, 1%, | Yes | No | 1 | | |
| | | 1%, 4% | | | | | |
| Sub- | sub-sub- | 10%, 5%, | Yes | Yes | 3 | | |
| group 3-2 | group 3-2-1 | 0.5%, 1% | | | | | |
| | | 10%, 5%, | Yes | Yes | 3 | | |
| | | 0.5%, 2% | | | | | |
| | | 10%, 5%, | Yes | Yes | 3 | | |
| | | 0.5%, 3% | | | | | |
| | | 10%, 5%, | Yes | Yes | 3 | 25.9 ± 15.0 | n.a. |
| | | 0.5%, 4% | | | | | |
| | sub-sub- | 10%, 5%, | Yes, but a | Yes | 3 | | |
| | group 3-2-2 | 1%, 1% | little | | | | |
| | | | viscous. | | | | |
| | | 10%, 5%, | Yes, but a | Yes | 3 | | |
| | | 1%, 2% | little | | | | |
| | | | viscous. | | | | |
| | | 10%, 5%, | Yes | Yes | 3 | | |
| | | 1%, 3% | | | | | |
| | | 10%, 5%, | Yes, but a | Yes | 3 | 25 ± 0 | 39 |
| | | 1%, 4% | little | | | | |
| | | | viscous. | | | | |
| Sub- | sub-sub- | 10%, 10%, | Yes | No | 3 | | |
| group 3-3 | group 3-3-1 | 0.5%, 1% | | | | | |
| | | 10%, 10%, | Yes, but | No | 3 | | |
| | | 0.5%, 2% | viscous. Got | | | | |
| | | | less viscous | | | | |
| | | | over time | | | | |
| | | 10%, 10%, | Yes, but | No, but held its | 3 | | |
| | | 0.5%, 3% | viscous. Got | shape for a | | | |
| | | | less viscous | little bit | | | |
| | | | over time | | | | |
| | | 10%, 10%, | Yes, but | No, but held its | 3 | | |
| | | 0.5%, 4% | viscous. Got | shape for a | | | |
| | | | less viscous | little bit | | | |
| | | | over time | | | | |
| | sub-sub- | 10%, 10%, | Yes, but | No | 3 | | |
| | group 3-3-2 | 1%, 1% | viscous. | | | | |
| | | 10%, 10%, | Yes | Yes | 3 | | |
| | | 1%, 2% | | | | | |
| | | 10%, 10%, | Yes | No | 3 | | |
| | | 1%, 3% | | | | | |
| | | 10%, 10%, | Yes | No | 3 | | |
| | | 1%, 4% | | | | | |
| Sub- | sub-sub- | 10%, 20%, | Yes | No | 3 | | |
| group 3-4 | group 3-4-1 | 0.5%, 1% | | | | | |
| | | 10%, 20%, | Yes, but | No | 3 | | |
| | | 0.5%, 2% | viscous | | | | |
| | | 10%, 20%, | No | No | 3 | | |
| | | 0.5%, 3% | | | | | |
| | | 10%, 20%, | No | Yes | 3 | | |
| | | 0.5%, 4% | | | | | |
| | sub-sub- | 10%, 20%, | No | No | 3 | | |
| | group 3-4-2 | 1%, 1% | | | | | |
| | | 10%, 20%, | Yes, but | No | 3 | | |
| | | 1%, 2% | viscous | | | | |
| | | 10%, 20%, | No | Yes | 3 | | |
| | | 1%, 3% | | | | | |
| | | 10%, 20%, | No | Yes | 3 | | |
| | | 1%, 4% | | | | | |

^{X}syringeability test results range from 1 to 5, where 1 is good syringeability (e.g., can be syringeable as liquid through a soft catheter without clogging) and 5 is poor syringeability (e.g., low ability to be syringeable as liquid through a soft catheter without clogging)

TABLE 4
Data summary for exemplary composition formulation optimization, group 4.

| | | | Gelation | Gelation | | | |
|---|---|---|---|---|---|---|---|
| | | | Test: | Test: | | Storage | |
| | | | Liquid | Turns | | modulus | Gelation |
| | | Solution | under room | Solid at body | Syringeability | at 37° C. | Temp |
| Group-4 | | Tested | temp.? | temp.? | Test:^{X} | (Pa) | (° C.) |
| Sub- | sub-sub- | 18%, 1%, | Yes (mostly) | Yes | 4 | | |
| group 4-1 | group 4-1-1 | 0.5%, 1% | | | | | |
| | | 18%, 1%, | Yes | Yes | 1 | | |
| | | 0.5%, 2% | | | | | |
| | | 18%, 1%, | Yes | Yes | 1 | | |
| | | 0.5%, 3% | | | | | |
| | | 18%, 1%, | Yes | Yes | 1 | 5429.0 ± 42.4 | 21 |
| | | 0.5%, 4% | | | | | |
| | sub-sub- | 18%, 1%, | Yes | Yes | 1 | | |
| | group 4-1-2 | 1%, 1% | | | | | |
| | | 18%, 1%, | Yes | Yes | 1 | | |
| | | 1%, 2% | | | | | |
| | | 18%, 1%, | Yes | Yes | 1 | | |
| | | 1%, 3% | | | | | |
| | | 18%, 1%, | Yes | Yes | 3 | 5049.8 ± 314.7 | 18 |
| | | 1%, 4% | | | | | |
| Sub- | sub-sub- | 18%, 5%, | Yes | Yes | 3 | | |
| group 4-2 | group 4-2-1 | 0.5%, 1% | | | | | |
| | | 18%, 5%, | Yes, but a | Yes | 3 | | |
| | | 0.5%, 2% | little viscous | | | | |
| | | 18%, 5%, | Yes | Yes | 3 | | |
| | | 0.5%, 3% | | | | | |
| | | 18%, 5%, | Yes, but a | Yes | 3 | 3589.7 ± 1142.3 | 16 |
| | | 0.5%, 4% | little viscous | | | | |
| | sub-sub- | 18%, 5%, | Yes | Yes | 3 | | |
| | group 4-2-2 | 1%, 1% | | | | | |
| | | 18%, 5%, | Yes, but a | Yes | 3 | | |
| | | 1%, 2% | little viscous | | | | |
| | | 18%, 5%, | Yes, but | Yes | 3 | | |
| | | 1%, 3% | viscous | | | | |
| | | 18%, 5%, | No | Yes | 1 | | |
| | | 1%, 4% | | | | | |
| Sub- | sub-sub- | 18%, 10%, | No | Yes | 3 | | |
| group 4-3 | group 4-3-1 | 0.5%, 1% | | | | | |
| | | 18%, 10%, | No | Yes | 3 | | |
| | | 0.5%, 2% | | | | | |
| | | 18%, 10%, | No | Yes | 3 | | |
| | | 0.5%, 3% | | | | | |
| | | 18%, 10%, | No | Yes | 4 | | |
| | | 0.5%, 4% | | | | | |
| | sub-sub- | 18%, 10%, | No | Yes | 3 | | |
| | group 4-3-2 | 1%, 1% | | | | | |
| | | 18%, 10%, | No | Yes | 4 | | |
| | | 1%, 2% | | | | | |
| | | 18%, 10%, | No | Yes | 3 | | |
| | | 1%, 3% | | | | | |
| | | 18%, 10%, | No | Yes | 4 | | |
| | | 1%, 4% | | | | | |
| Sub- | sub-sub- | 18%, 20%, | No | Yes | 4 | | |
| group 4-4 | group 4-4-1 | 0.5%, 1% | | | | | |
| | | 18%, 20%, | No | Yes | 3 | | |
| | | 0.5%, 2% | | | | | |
| | | 18%, 20%, | No | Yes | 3 | | |
| | | 0.5%, 3% | | | | | |
| | | 18%, 20%, | No | Yes | 3 | | |
| | | 0.5%, 4% | | | | | |
| | sub-sub- | 18%, 20%, | No | Yes | 3 | | |
| | group 4-4-2 | 1%, 1% | | | | | |
| | | 18%, 20%, | No | Yes | 4 | | |
| | | 1%, 2% | | | | | |
| | | 18%, 20%, | No | No | 4 | | |
| | | 1%, 3% | | | | | |
| | | 18%, 20%, | No | No | 3 | | |
| | | 1%, 4% | | | | | |

^{X}syringeability test results range from 1 to 5, where 1 is good syringeability (e.g., can be syringeable as liquid through a soft catheter without clogging) and 5 is poor syringeability (e.g., low ability to be syringeable as liquid through a soft catheter without clogging)

There are 32 exemplary composition formulations in each group (each of groups 1, 2, 3, and 4), categorized based on their polymer concentration (e.g., 10% PBP, 12% PBP, 15% PBP, 18% PBP; where “PBP” is poloxamer 407-poly(butoxy)phosphoester). Each group contains 32 composition formulations and is then divided into four sub-groups based on the concentration of SDS (e.g., 1% SDS, 5% SDS, 10% SDS, 20% SDS). Therefore, there are 8 formulations within each sub-group. These sub-groups are then divided first according to their bupivacaine concentration (low to high, sub-sub-group), then arranged according to their limonene concentration (low to high). Therefore, each sub-sub-group is composed of 4 formulations with the same PBP, SDS, and bupivacaine concentration, but different limonene concentrations. Within each sub-sub-group, the formulation with the highest limonene concentration and one that satisfies the following conditions on which to perform rheology was then chosen.

The selection conditions are: (A) liquid at room temperature (fourth column in Tables 1-4); (B) solid at body temperature (fifth column in Tables 1-4); and (C) good syringeability (sixth column in Tables 1-4) at room temperature. The reasonably viable exemplary compositions are italicized in Tables 1-4. The rheology data of these exemplary compositions are provided in the rightmost two columns of the table.

Among the samples on which rheology was performed, the ones satisfying the following conditions were selected for ex vivo experiments (testing trans-tympanic permeability): (1) a gelation temperature above room temperature and below body temperature (last column in Tables 1-4); (2) storage modulus at body temperature is over 100 Pa (second to last columns in Tables 1-4). (3) If there are two formulations in a sub-group (e.g., two formulations with the same PBP and SDS concentrations), which both satisfy (1) and (2) above, only the one with higher bupivacaine concentration is picked. The exemplary chosen formulations (well-performing formulations) are labelled in italics in Tables 1-4. 4 well-performing formulations were selected based on the data described herein.

### Experimental Procedures for Data in Tables 1-4

Experimental procedures for generating the data in Tables 1-4 above are as follows. To determine data for the fourth columns in Tables 1-4, the formulations were kept in a vial under lab ambient conditions (˜20-25° C.) for 1-5 minutes. The vials were then flipped over. If the formulation flowed down the side wall of the vial, then it was considered a liquid. To determine data for the fifth columns in Tables 1-4, the vials containing formulations were submerged in a 37° C. water bath for 30 seconds. The vials were then flipped over. If the formulation stayed on the bottom of the vial (flipped upside down), then it is considered a gel. To determine data for the sixth columns in Tables 1-4, the formulations (kept on ice) were drawn into 1-ml syringes. A 18-gauge, 1.88 inch soft catheter was then attached to each syringe, and the formulation was extruded through the catheter onto a glass surface (kept under lab ambient conditions). If the extruded material formed drops on the receiving surface, then it was considered syringeable. If the extruded material formed a rod-shaped solid, then the formulation was considered not syringeable.

The data in the last two columns in Tables 1-4 were calculated from rheology measurements, using the following conditions: The storage and loss moduli over the temperature range of 10-40° C. were measured in temperature ramp/sweep mode using linear oscillatory shear rheology. Oscillation rate of 100 rad per second, deformation strain rate of 1%, and temperature ramping rate of 1° C./min were used. Gelation temperature was considered to be the temperature where the storage modulus became greater than the loss modulus.

### Example 4

Here, the use of this trans-tympanic drug delivery system to deliver local anesthetics across the TM was also studied. Bupivacaine, an amphiphilic amino-amide local anesthetic in current clinical use, which has been found to have an intrinsic activity as a CPE, was studied. Tetrodotoxin (TTX), a very hydrophilic compound that blocks the same sodium channel as bupivacaine but at a different site, and has ultrapotent local anesthetic activity, was also studied. Bupivacaine and TTX are known to strongly increase each other's anesthetic effects when given in combination¹⁵⁻¹⁷.

### Materials

2-chloro-2-oxo-1,3,2-dioxaphospholane (COP), 1,8-diazabicyclo[5.4.0]undec-7-ene (DBU), n-butanol, diethyl ether, acetic acid, anhydrous dichloromethane, anhydrous tetrahydrofuran, SDS, LIM, and US pharmaceutical grade BUP and bupivacaine free base (BUP-fb) were used as received from Sigma-Aldrich (St. Louis, Mo.). US pharmaceutical grade TTX was used as received from Abcam Inc. (Boston, Mass.). US pharmaceutical grade Kolliphor® P407 micro-prilled (pelletized into micro-particles), received from BASF (Florham Park, N.J.).

### Animal Maintenance

Healthy adult male chinchillas weighting 500 to 650 g were purchased from Ryerson Chinchilla Ranch (Plymouth, Ohio) and cared for in accordance with protocols approved institutionally and nationally. Experiments were carried out in accordance with the Boston Children's Hospital Animal Use Guidelines and approved by the Animal Care and Use Committee.

### Synthesis of butoxy-2-oxo-1,3,2-dioxaphospholane (BP)

BP was prepared as reported previously¹⁴. Briefly, BP was synthesized by condensation reaction of COP and n-butanol. COP (5.0 g, 35 mmol) in anhydrous THF (50 mL) was added to a stirring solution of n-butanol (2.6 g, 35 mmol) and trimethylamine (3.9 g, 39 mmol) in anhydrous THF (100 mL) at 0° C. dropwise. The reaction mixture was stirred in an ice bath for 12 hours upon completed addition of COP in THF. Upon complete conversion of COP, the reaction mixture was filtered, and the filtrate was concentrated. The concentrated filtrate was purified by vacuum distillation under reduced vacuum to yield a viscous colorless liquid.

### Synthesis of P407-PBP

P407-PBP was synthesized as reported previously¹⁴, by ring opening polymerization (ROP) of BP with P407 as the macroinitiator in the presence of an organocatalyst, DBU at −20° C.¹⁸. P407 (8.1 g, 0.56 mmol) and BP (1.0 g, 5.6 mmol) in anhydrous dichloromethane (DCM, 0.5 mL) was added to a flame dried Schlenk flask (10 mL) equipped with a stir bar. The reaction mixture was flushed with nitrogen gas for 5 min while immersed in an ice bath with saturated NaCl solution. A solution of DBU in anhydrous DCM (0.13 g, 0.84 mmol) was added to the stirring solution via a syringe dropwise while maintaining the reaction under nitrogen gas atmosphere. Upon completion of the reaction, excess amount of acetic acid in DCM was added to the reaction mixture to quench the reaction. The product was purified by precipitation into ether (3 times) and dried under vacuum to obtain a white powder product.

### Hydrogel Formation

Solutions of 12% (w/v) P407-PBP hydrogel formulations were made by addition of powdered polymers to distilled and de-ionized water and simple dissolution in a cold room to allow better solubility of P407-PBP. SDS, and/or LIM, and/or BUP, and/or TTX were added to the solution of 12% (w/v) P407-PBP and allowed to dissolve in a cold room for at least 4 hours. The TTX hydrogel formulations were made with citrus buffer to enhance TTX solubility.

### In Vitro Release Studies

The release of BUP or TTX from each formulation was measured using a diffusion system. Transwell® membrane inserts (0.4 μm pore size, 1.1 cm2 area; Costar, Cambridge, Mass.) and 24-well culture plates were employed as the donor and acceptor chambers, respectively. 200 μL of each formulation was pipetted directly onto pre-warmed filter inserts to obtain a solid hydrogel. Filter inserts (donor compartments) with formed gels were suspended in wells (acceptor compartments) filled with pre-warmed phosphate buffered saline (PBS) and the plates then kept in a 37° C. incubator. At each time point (0.5, 1, 2, 6, 12, 24, 48 h), 1 mL aliquots of the PBS receiving media were sampled and inserts sequentially moved into a new well with fresh PBS. Aliquots were suspended in 70:30 acetonitrile/PBS to ensure total drug dissolution. Sample aliquots were chromatographically analyzed with high-performance liquid chromatography (HPLC) to determine BUP concentrations (absorption at the wavelength λ=254 nm); or analyzed with REAGEN™ TTX Elisa test kit (Reagen LLC. Collingswood, N.J.) to quantify TTX concentrations. Experiments were performed in quadruplicate.

### Ex Vivo Permeation Experiment

The trans-tympanic permeation rate of BUP and/or TTX was determined with auditory bullae harvested from healthy chinchillas. Chinchillas were placed under deep general anesthesia by the intramuscular administration of ketamine (30 mg/kg) and xylazine (4 mg/kg), and then euthanized with intracardiac administration of pentobarbital (100 mg/kg). Euthanized animals were decapitated and the auditory bullae removed undamaged, with the tympanic ring still attached. Their integrity was assessed by measuring their electrical impedance (indicated by a resistivity ≥18 kOhm*cm²; a value previously determined¹³) in a setup where TMs were placed horizontally in a 12-well plate with donor solution above and recipient solution below. The same setup was used to measure drug flux, in lieu of a conventional diffusion cell—which would deform or rupture the TM. All formulations were applied into the bullae kept at 37° C. and deposited onto the TMs. The concentration of BUP ranged from 0.5 to 15%, and the volume applied was 200 μL, which translates to 1-30 mg of BUP. The concentration of TTX was from 0.02% to 0.32% (solubility limit of TTX), and the volume applied was 200 μL, translating to 0.03 to 0.64 mg of TTX. The BUP and TX concentrations in the receiving chamber were measured at 0.5, 1.0, 2.0, 6.0, 12, 24 and 48 hours after the administration of the hydrogel compound. Permeation of BUP and/or TTX across TM into the receiving chamber was quantified using HPLC or TTX Elisa kit. Detailed information regarding TM harvesting, TM electrical resistance measurement, and configuration of the ex vivo permeation experiment can be found in reference¹³.

### Histopathology

Hydrogel formulations containing anesthetics and CPEs were administered to the ear canals of healthy chinchillas. Twenty-four hours to seven days later, they were euthanized as described above. Following sacrifice, the bullae were excised as described above to obtain samples of the TM and the external auditory meatus. Excised tissues were immediately fixed with 10% formalin overnight, then decalcified, embedded in paraffin, sectioned (10 um thick), and stained with hematoxylin and eosin. All stained specimens were evaluated by light microscopy in a blinded fashion.

### Statistical Analysis

For the ex vivo experiments, a sample size of 4 for each formulation was chosen, which would provide 80% power to detect 50% differences in flux based on power analysis using the nonparametric Friedman test (version 7.0, nQuery Advisor, Statistical Solutions, Saugus, Mass.). Statistical analysis was conducted using Origin 8 software (version 9.2, SAS Institute, Cary, N.C.). Data were presented as median (1st quartile-3rd quartile).

### Calculation of Hypothetical Drug Levels in Middle Ear Fluid

The following assumptions were made in order to calculate the middle ear concentrations of bupivacaine and TTX that would be achieved in vivo: (1) the fluid turnover rate is zero in the middle ear of AOM patients (i.e. middle ear fluid is not replenished), because middle ear fluid drainage is impeded by inflammation of the Eustachian tube mucosa in AOM 19; (2) drug concentration changes due to absorption by the surrounding middle ear mucosa, digestion by bacteria and enzymes, etc. are negligible; (3) the average volume of the human middle ear is ˜0.45 mL 20; (4) infinite sink conditions, which were applied during ex vivo experiments where the receiving chamber volume is 3 mL, still hold true for the human middle ear volume of 0.45 mL.

The measured cumulative mass of drug to have crossed the TM at any time point was divided by the volume of the human middle ear (0.45 mL) to provide the concentration that could have been achieved by a given formulation.

### Results

### Overview and Nomenclature of the Formulation

Hydrogel formulations were made in aqueous solutions of the penta-block copolymer P407-PBP at 12% (w/v), with or without additional CPEs, with or without the local anesthetics BUP [0.5 to 15% (w/v); concentrations above 4% (w/v) were suspensions, which were labeled with the subscript susp] and/or TTX [0.02 to 0.32% (w/v)]. When CPEs were added, the composition was 1% (w/v) SDS with 2% (w/v) LIM; this combination was referred to as 2CPE. The gels are referred to as x % BUP(susp)-y % TTX-2CPE-[P407-PBP], where x and y are the weight by volume percentage concentrations of BUP and TTX respectively. Twelve percent P407-PBP was used throughout this work as it was easily extruded from a syringe at room temperature and gelled rapidly at body temperature¹⁴. (The latter property would be important when applying the materials in toddlers who prefer not to stay still. The hydrogel is necessary for the continuous exposure of TMs to CPEs and anesthetics¹⁴.) If a component was absent from a formulation, it was omitted from the above nomenclature. Unless specified otherwise, all percentages are weight by volume percent.

The formulation containing BUP dissolved in pure LIM was referred to as x % BUP-LIM, where x was the weight by volume percentage concentration of BUP.

P407-PBP was synthesized by ring-opening polymerization, as reported¹⁴. Nuclear magnetic resonance (NMR) confirmed the presence of the PBP moieties and determined the degree of polymerization of the PBP moieties to be 5. Fourier transform infrared spectroscopy (FTIR) confirmed the successful synthesis of the penta-block copolymer P407-PBP.

### Effect of BUP Concentration on Trans-Tympanic Permeation Rate

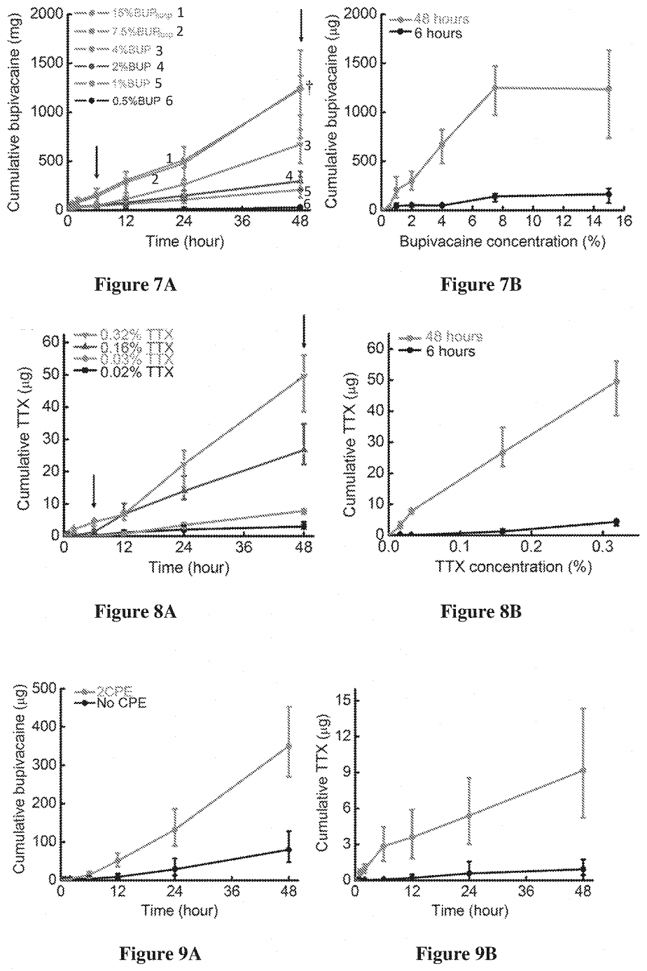

The trans-tympanic permeation rate of BUP was assessed using a previously reported ex vivo method¹⁴. In brief, drug transport across the TM was studied at 37° C. using auditory bullae excised from healthy chinchillas. 200 μL of anesthetic formulations (donor solution) were placed on one surface of the TM (see Methods for details) and flux into 3 mL of PBS (recipient solution) was measured over time (FIG. 7).

Flux of BUP across the TM from BUP-2CPE-[P407-PBP] formulations was studied in the BUP concentration range 0.5% to 15% (FIG. 7). Note that BUP was only soluble at concentrations up to 2% in water, and up to 4% in 12%[P407-PBP] solution. Therefore, the formulations of 7.5% BUPₛᵤₛₚ-2CPE-[P407-PBP] and 15% BUPₛᵤₛₚ-2CPE-[P407-PBP] were suspensions of dissolved and solid BUP. BUP flux increased continuously with increasing BUP concentration up to ˜7.5%.

At 6 hours, BUP permeation across the TM in the presence of 2CPE was about 1.5 μg (1.1-1.9 μg) for 0.5% BUP-2CPE-[P407-PBP] (FIG. 7). Increasing BUP concentration from 0.5% to 1% improved the trans-tympanic flux of BUP by about 28-fold, yielding a 6-hour BUP cumulative permeation of 42.7 μg (27.4-71.7 μg). Further increasing BUP concentration to 2% or 4% did not yield much improvement in BUP flux, with 2% BUP-2CPE-[P407-PBP] achieving 51.0 μg (35.3-68.1 μg) and 4% BUP-2CPE-[P407-PBP]achieving 48.0 μg (43.9-51.2 μg). The suspension, 7.5% BUPₛᵤₛₚ-2CPE-[P407-PBP], further increased 6-hour BUP cumulative permeation to 141.1 μg (85.6-168.8 μg); there was no further increase with 15% BUPₛᵤₛₚ-2CPE-[P407-PBP] [163.6 μg (74.3-223.2 μg)].

At 48 hours, increasing the BUP concentration from 0.5% to 1% increased the BUP flux from 27.0 μg (19.4-31.5 μg) to 208.1 μg (127.7-340.8 μg), a 8-fold enhancement (FIG. 7B). Further increasing the BUP concentration to 2% yielded a small increase in BUP flux, to 296.4 μg (206.1-395.7 μg). Doubling the BUP concentration again, to 4%, achieved another 2-fold increase in BUP flux, to 671.9 μg (479.4-820.9 μg). The maximum cumulative permeation of BUP was achieved with 7.5% BUPₛᵤₛₚ-2CPE-[P407-PBP], which resulted in 1251.2 μg (971.5-1471.0 μg) BUP crossing the TM by 48 hours. The quantity of BUP that permeated across the intact TM corresponded to ˜8.3% of the total BUP applied on the TM. Increasing the BUP concentration from 7.5% to 15% did not yield any further enhancement of 48-hour permeation.

### Effect of TTX Concentration on Trans-Tympanic Permeation Rate

Flux of TTX across the TM was evaluated by the same ex vivo method. The concentration of TTX by Enzyme-Linked Immunosorbent Assay (ELISA) (See Methods for details). The concentration of TTX in TTX-2CPE-[P407-PBP], was varied from 0.02% (0.5 mM) to 0.32% (10 mM, FIG. 8), where 0.32% (10 mM) was the solubility limit of TTX.

At 6 hours, trans-tympanic permeation of TTX increased roughly linearly with the TTX concentration in the formulation (FIG. 8B). Increasing TTX concentration from 0.02% (0.5 mM) to 0.16% (5 mM, i.e. 10-fold) resulted in a 6-fold increase of TTX permeability, from 0.2 μg (0.2-0.3 μg) to 1.3 μg (0.9-2.0 μg). Doubling the TTX concentration from 0.16% (5 mM) to 0.32% (10 mM) resulted in another 3-fold increase of TTX permeability, from 1.3 μg (0.9-2.0 μg) to 4.4 μg (3.2-5.1 μg). At 48 hours, the linear correlation remained between TTX concentration and trans-tympanic permeability, where 0.02% TTX-2CPE-[P407-PBP] led to 3.0 μg (2.3-4.4 μg) cumulative permeation of TTX, and 0.03%, 0.16%, and 0.32% TTX formulations achieved 3-, 9- and 16-fold enhancement respectively.

### Formulations Combining BUP and TTX

Combining BUP and TTX has been shown to enhance anesthetic effect dramatically^{15-17,21}. Here, the concentration of BUP in the combined formulation was fixed at 2%. The TTX concentration was kept constant at 0.03% (1 mM) because similar concentrations have been used topically^{22,23}. The trans-tympanic permeability of BUP and TTX was studied in the ex vivo model described above, from 2% BUP-0.03% TTX-[P407-PBP] and 2% BUP-0.03% TTX-2CPE-[P407-PBP] (FIG. 9).

At 6 hours, only 4.3 μg (0.6-10.8 μg) BUP permeated across the TM from 2% BUP-0.3% TTX-[P407-PBP]. Incorporating 2CPE into the formulation led to a 3-fold increase of BUP trans-tympanic permeation. The enhancement effect of 2CPE on TTX permeation was much greater—29 fold, from 0.1 μg (0-0.2 μg) to 2.9 μg (1.6-4.5 μg).

At 48 hours, the cumulative permeation of BUP achieved by 2% BUP-0.3% TTX-[P407-PBP] was ˜80.2 μg (47.7-128.1 μg), ˜2.0% of the total applied BUP (FIG. 9A); the cumulative permeation of TTX was ˜0.9 μg (0.4-1.7 μg), ˜1.4% of the total applied TTX (FIG. 9B). Incorporating 2CPE increased the trans-tympanic BUP permeation to 350.2 μg (270.1-452.9 μg), ˜8.8% of the total amount (4 mg) of BUP applied on the TM (FIG. 9A). During the same period, 9.2 μg (5.2-14.4 μg) TTX permeated across the TM, corresponding to 14.3% of the total amount of applied TTX (63.9 μg, FIG. 9B). The 2CPE combination increased permeability of BUP 4-fold and that of TTX 10-fold.

### Terpene-Based Anesthetic Formulations

In all of the preceding sections, bupivacaine hydrochloride (BUP) was used to formulate the anesthetic hydrogel because of its hydrophilicity. Nonetheless, the highest soluble concentration was 4%. Increasing the concentration of SDS and/or LIM (the 2CPE) up to their respective solubility limits of 20% and 10% did not improve BUP solubility in water. BUP solubility in water was not affected by tuning the pH of the formulation in the range of 3 to 9 to alter the proportion of bupivacaine in the salt form [higher at lower pH] and the more hydrophobic free base.

To increase the soluble BUP concentration in the formulation, bupivacaine free base (BUP-fb) was used instead, and dissolved in pure LIM. Pure LIM was chosen as the solvent because of its hydrophobicity²⁴, its proven permeation enhancement effect^{13,14,25} and its FDA-approved status for topical applications. The solubility limit of BUP-fb is ˜10% in pure LIM, the highest soluble bupivacaine concentration established thus far.

Using 10% BUP-fb-LIM in the above ex vivo flux model, the cumulative amount of BUP-fb delivered into the middle ear was 63.5 μg (45.3-68.9 μg) after 0.5 hours. The middle ear drug level increased 3-, and 27-fold after 6 and 48 hours (FIG. 10). The trans-tympanic drug permeability achieved by 10% BUP-fb-LIM [1709.8 μg (1600.1-1742.5 μg)] was not significantly different from that of 15% BUPₛᵤₛₚ-2CPE-[P407-PBP] [1234.5 μg (735.5-1633.8 μg)].

### In Vivo Biocompatibility in the Ear

Biocompatibility in the ear was tested by treating healthy chinchillas with the anesthetic-containing formulations, followed by histopathology evaluation of the treated ears (see Section 2.8 for experimental details). For the hydrogel formulations, the duration of the treatment was set to 7 days, a typical treatment duration for acute otitis media². For 10% BUP-fb-LIM, the exposure time 24 hours because of the clinically apparent inflammatory reactions by that time. The inflammatory tissue reactions disappeared after 7 days.

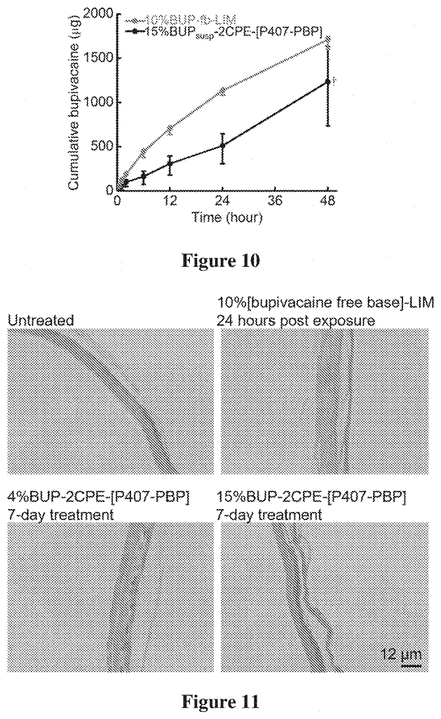

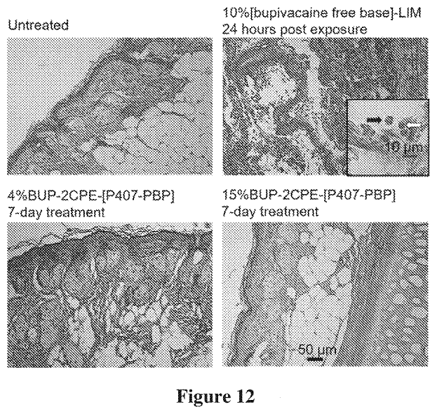

In animals treated with 4% BUP-2CPE-[P407-PBP] or 15% BUPₛᵤₛₚ-2CPE-[P407-PBP] for 7 days, hematoxylin-eosin-stained sections of the TMs looked similar to normal (FIG. 11). No inflammation, necrosis, or tissue damage was observed. Moreover, the external auditory meatus of the treated animals looked similar to healthy meatuses in the hematoxylin-eosin-stained sections (FIG. 12). The sections showed normal epithelium (outermost layer), covering normal adnexal structures/glands, with no inflammation.

Healthy TMs treated with 10% BUP-fb-LIM for 24 hours looked similar to the normal ones (FIG. 11). However, a severe acute and chronic inflammatory response was observed in the external auditory meatus of the treated animals (FIG. 12). The inflammatory response consisted of lymphocytes, monocytes, and neutrophils in the epidermis and subepidermal layers of treated animals. In addition, animals that received 10% BUP-fb-LIM exhibited behavioral anomalies such as excessively scratching their treated ears.

### DISCUSSION

The hydrogel drug delivery system achieved trans-tympanic delivery of bupivacaine and TTX in a sustained manner. The formulation containing both anesthetics, 2% BUP-0.3% TTX-2CPE-[P407-PBP], delivered 350.2±102.7 μg BUP and 9.2±5.2 μg TTX across the TM in 48 hours. That corresponds to an average flux of ˜7.3 μg/h for bupivacaine and ˜0.2 μg/h for TTX.

The drug concentrations that might occur in humans from the fluxes stated above were calculated as described in Methods. After 6 hours of exposure to 2% BUP-0.3% TTX-2CPE-[P407-PBP], the cumulative flux of drug was such that the bupivacaine concentration in the middle ear could reach 0.03 mg/mL (dividing the cumulative flux of 0.013 mg by 0.45 mL; i.e. 0.09 mM) and the tetrodotoxin concentration 6.4 μg/mL (dividing the cumulative flux of 2.9 μg by 0.45 mL; i.e. 20 μM) TTX. At 48 hours, the drug concentrations increased to ˜0.8 mg/mL (dividing the cumulative flux of 0.35 mg by 0.45 mL; i.e. 3 mM) for BUP and ˜0.02 mg/mL (dividing the cumulative flux of 9.2 μg by 0.45 mL; i.e. 64 μM) for TTX.

The concentrations measured in the receiving chamber are the product of drug penetrating throughout the tissue and then exiting, i.e. they reflect the concentrations in the tissue. In considering whether these concentrations would achieve local pain relief, it is useful to first consider what concentrations would result in local anesthesia in tissue. In vitro, bupivacaine inhibits most sodium current with a KI=25 μM²⁶, and reduces the amplitudes of action potentials with a median inhibitory concentration of 180 μM²⁷; the corresponding values of TTX are 1-2 nM^{28,29} and 5-6 nM³⁰. The concentrations in the receiving chamber all were higher than the nano- to micromolar concentrations required for nerve block in vitro. For bupivacaine, the concentrations in the receiving chamber were also much higher than the blood (systemic) drug concentrations required to achieve analgesia in animals. A plasma lidocaine concentration of 0.36 μg/mL (1.5 μM) achieved analgesia in a rat neuropathic pain model³¹; this was 1.2% the bupivacaine concentration achieved here at 6 hours, and 0.05% the concentration at 48 hours. (In addition, bupivacaine is ˜4 times more potent than lidocaine³².) The concentrations of TTX achieved at 6 and 48 hours here are actually concentrations that achieve nerve block (tens of μM) when used in perineural block^{33,34}.

The flux of bupivacaine and TTX across the TM would likely be even greater had tympanic membranes from animals with OM been used here instead of tympanic membranes from healthy animals. In OM, The tympanic membrane becomes much more permeable to drug flux even though it also become much thicker¹⁴. That greater drug flux could markedly enhance drug levels in the middle ear.

Moreover, local anesthetic efficacy could be greatly enhanced were bupivacaine and TTX to be co-delivered¹⁵⁻¹⁷. Conventional amino-amide or amino-ester local anesthetics such as bupivacaine are known to have marked synergy with compounds such as tetrodotoxin, which block the same sodium channel at a different site termed site 1 on the axonal surface. Concentrations of either compound that would be relatively ineffective independently can become effective in combination. Moreover, CPEs are known to enhance the local anesthetic effect of tetrodotoxin, presumably by enhancing penetration to the axon surface 3-37.

The effectiveness of ear drops containing anesthetics such as lidocaine is controversial, and is short-lived³⁸; this poor performance is likely due to the well-known barrier function of the tympanic membrane¹². The permeation barrier was overcome, and therapeutic levels of bupivacaine and TTX were delivered across intact tympanic membranes. In addition, the hydrogel extended the effect over a prolonged period that would likely cover the time frame within which otalgia is at its worst. This would likely be even more effective with dual delivery of conventional local anesthetics and site 1 sodium channel blockers, since co-delivery can markedly enhance the duration of effect^{15-17,33}.

Although CPEs increased the trans-tympanic flux of both BUP and TTX, the effect on the flux of TTX (a 10-fold increase at 48 hours) was much greater than that on BUP (a 4-fold increase at 48 hours). This pattern was reminiscent of the effect of CPEs co-injected with those compounds at the sciatic nerve³⁵: nerve blockade by TTX was markedly enhanced by CPEs, while that from BUP was not. It was possible that the reason for this difference was that TTX, being very hydrophilic, had great difficulty penetrating biological barriers, and so would benefit from the CPEs. BUP, being amphiphilic, would have less trouble penetrating biological barriers, and so would benefit less from the CPEs.

Although 10% BUP-fb-LIM had a greater dissolved drug concentration than the hydrogel formulations, the trans-tympanic permeation of BUP was similar. 10% BUP-fb-LIM achieved a BUP concentration of ˜0.4 mg/mL (1.2 mM) in the middle ear at 6 hours after administration. 10% BUP-fb-LIM caused a severe inflammatory response in the meatus, which could be a result of the high LIM concentration or the high free bupivacaine concentration in the formulation. The inflammatory response was not seen in the TM, presumably because in the absence of the hydrogel, the 10% BUP-fb-LIM flowed off of the TM into the auditory canal once the animals woke up.

It was interesting that the hydrogels containing suspensions of bupivacaine, such as 7.5% BUPₛᵤₛₚ-2CPE-[P407-PBP], increased the trans-tympanic permeation of bupivacaine by 2-fold at 48 hours compared to hydrogel solutions such as 4% BUP-2CPE-[P407-PBP], since the concentrations of bupivacaine is solution were presumably the same. It is possible that the drug in suspension acted as a drug reservoir replenishing the concentration of free drug on the TM surface as it was depleted by flux.

It has previously been shown, using a similar hydrogel delivery system, that trans-tympanic drug delivery results in no detectable systemic (blood) distribution of the antibiotic ciprofloxacin^{14,39}. Presumably, trans-tympanic delivery of bupivacaine and TTX would also not result is systemic drug distribution, and so would obviate the side effects of the local anesthetics. This treatment would also obviate the need for systemic (oral) analgesics and their potential side effects.

The thermosensitive hydrogel was designed to provide sustained pain relief and enable easy administration. The hydrogel formulation is a solution under room temperature for administration through the ear canal like other regular ear drops; the formulation gels quickly in situ upon contacting the warm TM. Only a single application is required to maintain local anesthesia over prolonged periods, which is beneficial because multi-dose regimens can cause poor compliance among uncooperative young patients.

A local drug delivery system was developed to provide sustained pain relief from a single application in patients with AOM. A commonly used amino-amide anesthetic, bupivacaine, was successfully delivered across intact TMs, as was a highly potent site 1 sodium channel blocker anesthetic, TTX. The chemical permeation enhancers incorporated in the hydrogel system considerably increased the permeability of BUP and TTX across the TM.

### Example 3

Chemical permeation enhancers (CPEs) can enable antibiotic flux across the tympanic membrane. Here it is investigated whether combinations of CPEs (sodium dodecyl sulfate, limonene, and bupivacaine hydrochloride) are synergistic and whether they could increase the peak drug flux. Synergy is studied by isobolographic analysis and combination indices. CPE concentration-response (i.e. trans-tympanic flux of ciprofloxacin) curves are constructed for each CPE, isobolograms constructed for pairs of CPEs, and synergy demonstrated for all three pairs. Synergy is much greater at earlier (6 hours) than later (48 hours) time points, although the effect sizes are greater later. Synergy is also demonstrated with the three-drug combination. Combinations of CPEs also greatly enhance the maximum drug flux achievable over that achieved by individual CPEs.

### Introduction

Ototopical drug delivery presents a promising alternative to oral therapeutics for drug administration to the middle ear. Localized delivery of therapeutics across the intact tympanic membrane (TM) and directly to the middle ear could minimize adverse systemic effects (diarrhea, rashes, and perhaps antibiotic resistance caused by oral antibiotics for the treatment of otitis media [OM] [44]), improve patient adherence with therapy (due to reduced side effects and obviation of the need for extended treatment of often uncooperative toddlers), and therefore possibly achieve better therapeutic outcomes. However, non-invasive trans-tympanic delivery has seldom been explored until recently [45,46] due to the impermeability of the TM. [47,48] The TM is a 100 m-thick trilayer membrane whose outer layer, the stratum corneum (SC), is a stratified squamous keratinizing epithelium continuous with the skin of the external auditory canal, and is structurally similar to that in skin.

Chemical permeation enhancers (CPEs) are an effective means of enhancing the flux of small-molecule therapeutics across the TM. [45,46] Moreover, the enhancement can be increased by increasing the concentration of CPEs. [49,50] CPEs are known to disrupt the structural integrity of the lipid bilayers in the stratum corneum, enhancing the diffusion of therapeutics. [49] It has previously been demonstrated that OM can be treated by the trans-tympanic delivery of ciprofloxacin (Cip) enabled by a combination of CPEs. [45,46] However, the benefits of combinations of CPEs remains to be demonstrated formally, specifically whether their effects are truly synergistic, or simply additive. Synergistic interactions hold the potential to reduce the amount of CPEs needed to achieve a given effect, thus potentially also reducing toxicity.

A related important issue is whether combinations of CPEs can be used to maximize peak effect, i.e. the maximum drug flux across a barrier. The magnitude of drug flux is particularly important in treating OM, as relatively high antibiotic concentrations are needed to treat some bacteria, such as the common OM pathogen Streptococcus pneumoniae. [51,52]

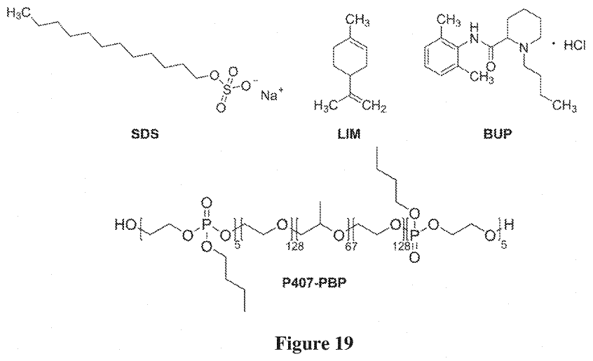

Pioneering work on interactions of CPEs has demonstrated the possibility of achieving higher than expected permeation enhancement when CPEs were combined. [53-58] Here, formal pharmacological approaches have been used to establish whether the CPE interactions noted here are synergistic [59] and also whether CPE combinations could be used to increase the peak effects that could be achieved. Potentially synergistic effects are investigated among three CPEs delivered in a polymer matrix (FIG. 19) in enhancing trans-tympanic permeation using isobole analysis [60,61] and combination indices. [59,62,63]

Sodium dodecyl sulfate (SDS), a surfactant, and limonene (LIM), a terpene, were chosen because they are both CPEs approved by the FDA for topical use. [64] SDS (an anionic surfactant) can enhance SC permeability by extracting lipids from the SC and altering the protein structure of keratin in corneocytes, [65] while LIM (a terpene) can partition into the SC lipids, forming a pathway for drug molecules. [66] Synergistic effects are often found with processes that act on a common phenomenon by different mechanisms. [53,66-68] The clinically-used local anesthetic bupivacaine hydrochloride (BUP) was studied because it may reduce pain associated with OM.

The effect of SDS, LIM, BUP, and their combinations on permeation enhancement was elucidated by measuring their effect on the permeability of Cip across the TMs of healthy chinchillas. Cip was selected because it is FDA-approved to be administered locally to the middle ear for the treatment of OM. [69] Cip and the CPEs were delivered from a hydrogel reported previously, poloxamer 407-polybutylphosphoester (P407-PBP) (FIG. 19).

P407-PBP was used here because of its robust reverse thermal gelation behavior. [45] The hydrogel-based formulation is an easy-to-apply liquid at room temperature, and gels quickly and firmly upon contacting the warm TM, holding the antibiotic and CPEs in place (i.e. on the TM) throughout the permeability measurements.

Chinchilla TMs were used as the model system here, because of their well-established structural similarity to human TMs [70]. The principal difference between chinchilla and human TMs is that the latter are much thicker human ones [45,71]. Nonetheless, the conclusions reached here are likely to bear on human TMs as well because a) the TMs in the two species are structurally similar and b) CPEs can have their effect even with much thicker structures, such as human skin.

### Materials and Methods

### Materials

2-chloro-2-oxo-1,3,2-dioxaphospholane (COP), 1,8-diazabicyclo[5.4.0]undec-7-ene (DBU), n-butanol, diethyl ether, acetic acid, anhydrous dichloromethane, anhydrous tetrahydrofuran, SDS, LIM, and US pharmaceutical grade Cip and BUP were used as received from Sigma-Aldrich (St. Louis, Mo.). Kolliphor® P407 micro-prilled (pelletized into micro-particles), received from BASF (Florham Park, N.J.).

### Animal Maintenance

Healthy adult male chinchillas weighting 500 to 650 g were purchased from Ryerson Chinchilla Ranch (Plymouth, Ohio) and cared for in accordance with protocols approved institutionally and nationally. Experiments were carried out in accordance with the Boston Children's Hospital Animal Use Guidelines and approved by the Animal Care and Use Committee.

### Synthesis of butoxy-2-oxo-1,3,2-dioxaphospholane (BP)

BP was prepared by condensation reaction of COP and n-butanol. COP (5.0 g, 35 mmol) in anhydrous THF (50 mL) was added to a stirring solution of n-butanol (2.6 g, 35 mmol) and trimethylamine (3.9 g, 39 mmol) in anhydrous THF (100 mL) at 0° C. dropwise. The reaction mixture was stirred in an ice bath for 12 hours upon completed addition of COP in THF. Upon complete conversion of COP, the reaction mixture was filtered and the filtrate was concentrated. The concentrated filtrate was purified by vacuum distillation under reduced vacuum to yield a viscous colorless liquid.

### Synthesis of P407-PBP

P407-PBP was synthesized by ring opening polymerization (ROP) of BP with P407 as the macroinitiator in the presence of an organocatalyst, DBU at −20° C. [30]. P407 (8.1 g, 0.56 mmol) and BP (1.0 g, 5.6 mmol) in anhydrous dichloromethane (DCM, 0.5 mL) was added to a flame dried Schlenk flask (10 mL) equipped with a stir bar. The reaction mixture was flushed with nitrogen gas for 5 min while immersed in an ice bath with saturated NaCl solution. A solution of DBU in anhydrous DCM (0.13 g, 0.84 mmol) was added to the stirring solution via a syringe dropwise while maintaining the reaction under nitrogen gas atmosphere. Upon completion of the reaction, excess amount of acetic acid in DCM was added to the reaction mixture to quench the reaction. The product was purified by precipitation into ether (3 times) and dried under vacuum to obtain a white powder product.

### Hydrogel Formation

Hydrogel solutions of 12% (w/v) P407-PBP hydrogel formulations were made by addition of powdered polymers to aqueous solutions of 4% (w/v) Cip (pH=3.3-3.9) and simple dissolution in a cold room to allow better solubility of P407-PBP. SDS, and/or LIM, and/or BUP were added to the solution of 4% (w/v) Cip and 12% (w/v) P407-PBP and allowed to dissolve in a cold room for at least 4 hours.

### In Vitro Release Studies

The release of Cip from each formulation was measured using a diffusion system. Transwell® membrane inserts (0.4 μm pore size, 1.1 cm2 area; Costar, Cambridge, Mass.) and 24-well culture plates were employed as the donor and acceptor chambers, respectively. 200 μL of each formulation was pipetted directly onto pre-warmed filter inserts to obtain a solid hydrogel. Filter inserts (donor compartments) with formed gels were suspended in wells (acceptor compartments) filled with pre-warmed phosphate buffered saline (PBS) and the plates then kept in a 37° C. incubator. At each time point (0.5, 1, 2, 6, 12, 24, 48 h), 1 mL aliquots of the PBS receiving media were sampled and inserts sequentially moved into a new well with fresh PBS. Aliquots were suspended in 70:30 acetonitrile/PBS to ensure total drug dissolution. Sample aliquots were chromatographically analyzed with high-performance liquid chromatography (HPLC) to determine Cip concentrations (absorption at the wavelength λ=275 nm). More details regarding the Cip measurement and HPLC conditions can be found in reference [46]. Experiments were performed in quadruplicate.

### Ex Vivo Permeation Experiment

The trans-tympanic permeation rate of Cip was determined with auditory bullae harvested from healthy chinchillas. Chinchillas were placed under deep general anesthesia by the intramuscular administration of ketamine (30 mg/kg) and xylazine (4 mg/kg), and then euthanized with intracardiac administration of pentobarbital (100 mg/kg). Euthanized animals were decapitated and the auditory bullae removed undamaged, with the tympanic ring still attached. Their integrity was assessed by measuring their electrical impedance (indicated by a resistivity ≥18 kOhm*cm2; a value previously determined [46]) in a setup where TMs were placed horizontally in a 12-well plate with donor solution above and recipient solution below. The same setup was used to measure drug flux, in lieu of a conventional diffusion cell—which would deform or rupture the TM. All formulations were applied into the bullae kept at 37° C. and deposited onto the TMs. The volume applied was 200 μL, which translates to 8 mg Cip. Permeation of Cip across TM into the receiving chamber was quantified using HPLC. Detailed information regarding TM harvesting, TM electrical resistance measurement, and configuration of the ex vivo permeation experiment can be found in reference [46].

### Statistical Analysis

Data which were normally distributed were described with means and standard deviations (calculated using Microsoft® Excel®) and compared by unpaired Student t-tests (using Origin® 8, OriginLab). Otherwise, data were presented as median±quartiles (using Microsoft® Excel®).

### Results

### Overview and Nomenclature of the Formulation

Hydrogel formulations were formulated with the antibiotic Cip at 4% (w/v), the penta-block copolymer P407-PBP at 12% (w/v), and CPEs at various concentrations; the gels are referred to as CPPB-x % LIM-y % SDS-z % BUP, where CPPB represents the invariant 4% Cip-12%[P407-PBP]; x, y, z are weight by volume percentage concentrations of LIM, SDS, and BUP respectively. Twelve percent P407-PBP was used throughout this work as it was easily extruded from a syringe at room temperature and gelled rapidly at body temperature. [45] (The latter property would be important when applying the materials in toddlers who prefer not to stay still. The hydrogel itself would maintain the antibiotic and CPEs at the TM in vivo. P407-PBP is necessary for the continuous exposure of TMs to CPEs and antibiotics. [45])

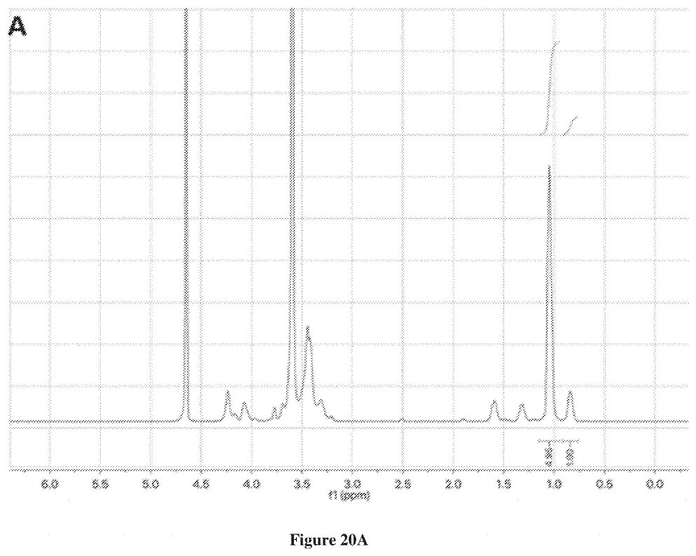

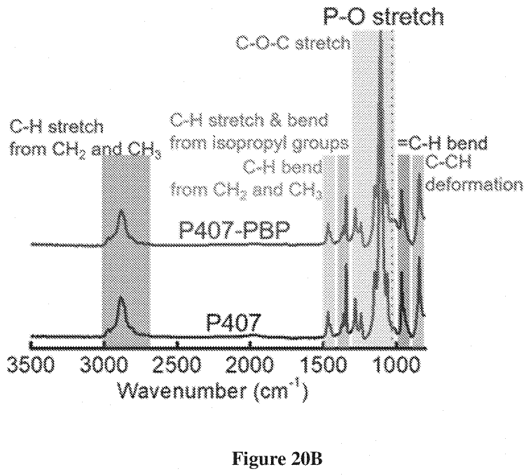

P407-PBP was synthesized by ring-opening polymerization, as reported. [45] Nuclear magnetic resonance (NMR) confirmed the presence of the PBP moieties and determined the degree of polymerization of the PBP moieties to be 5 (FIG. 20A). Fourier transform infrared spectroscopy (FTIR) confirmed the successful synthesis of the penta-block copolymer P407-PBP (FIG. 20B).

If a component was absent from a formulation, it was omitted from the above nomenclature. A previously reported combination of three CPEs, [45] i.e., 2% LIM, 1% SDS, and 0.5% BUP is denoted as 3CPE. Unless specified otherwise, all percentages are weight by volume percent.

The cumulative amount of Cip that permeated across excised TM in ex vivo experiments, was represented as VCIPt, where t is the time in hours over which cumulative permeation of Cip was measured. Specifically, VCIP6 and VCIP48 represent the cumulative amount of Cip that permeated across the TM within 6 and 48 hours in ex vivo experiments, respectively.

In Vitro Drug Release from Hydrogels

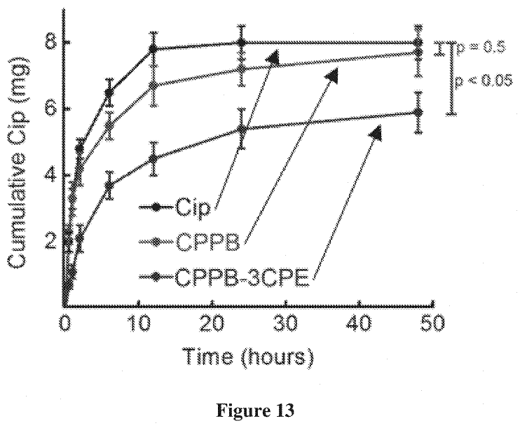

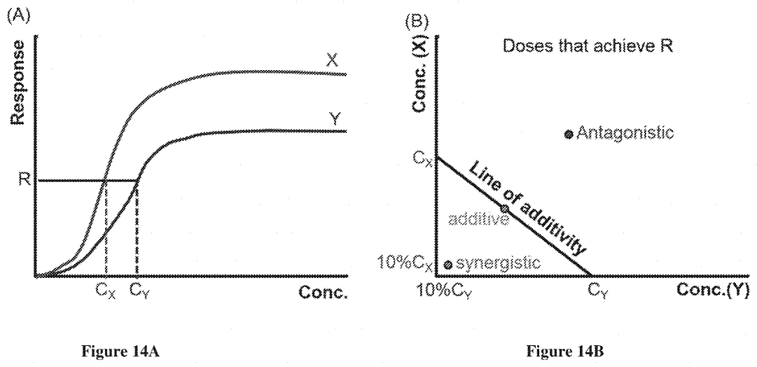

The release of Cip from each formulation was measured using Transwell® membrane inserts. Cip release from 200 μL of CPPB gels containing 8.0 mg of drug with or without CPEs was measured at 37° C. (FIG. 13). Drug release slowed down significantly after roughly 12 hours for Cip solution, and roughly 24 hours for CPPB gels with or without CPEs. In 48 h, CPPB released almost the entirety of the loaded Cip (7.7 mg), while CPPB-3CPE released approximately three quarters (5.9 mg).

### Synergistic Interactions Among CPEs

### Isobolographic Analysis

A key concept in comparing interactions of drug doses is that of dose equivalence. [60,61] One rigorous way of establishing equivalence is in terms of a dose that affects a given percentage of a population or has a given percentage of a maximal effect (both of these have been defined as, for example, the EC50 [half maximal effect concentration]). In such cases, the effects of doses can be compared by isobolographic analysis.

The following steps are followed to perform the isobolographic analysis. Concentration-response curves are constructed for drugs X and Y, and the equivalent concentration (or dose) to achieve a given effect (e.g., the VCIP48 of 0.4 mg) is determined for each (FIG. 14A). An isobologram (FIG. 14B) is constructed where the concentration of drug X to achieve that given effect is plotted on the x-axis and the equivalent for drug Y on the y-axis. A line connecting the two (the isobole) is the line of additivity; the effect of combinations of fractions of the equivalent doses for drugs X and Y are then plotted on the graph. If, for example, a combination of 10% of the equivalent dose of X and 90% of the equivalent dose of Y (i.e. a total of 100% of an equivalent dose) achieves the given effect, then X and Y are simply additive. If only 10% of the equivalent dose of X and 10% of the equivalent dose of Y (i.e. 20% of an equivalent dose) achieve the given effect, they are synergistic. If a combination of 90% of the equivalent dose of X and 90% of the equivalent dose of Y (i.e. 180% of an equivalent dose) have the given effect they are antagonistic.

### Concentration-Response Curves for Single CPEs

To produce the isobolographic analysis, curves were generated (analogous to FIG. 14) relating the effect of concentrations of single CPEs to trans-tympanic drug permeation of Cip. These curves were subsequently used to construct isobolograms [61] to assess whether the effects of combinations of CPEs were additive, synergistic, or possibly antagonistic.

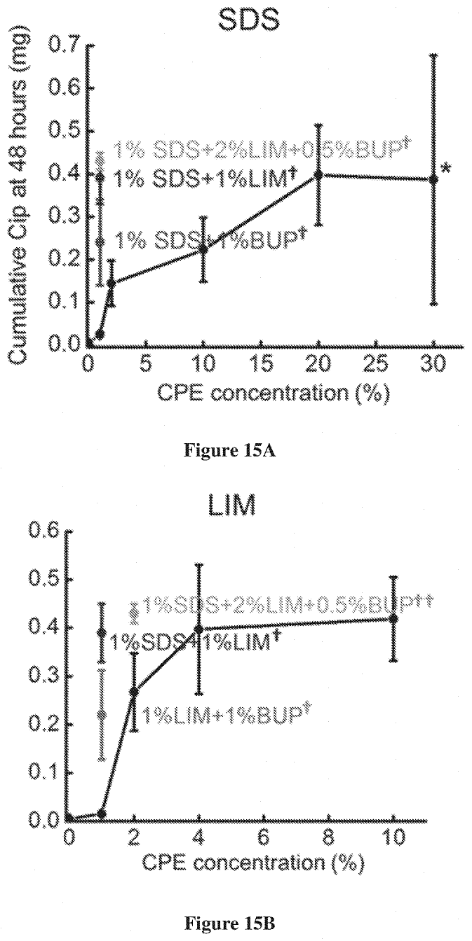

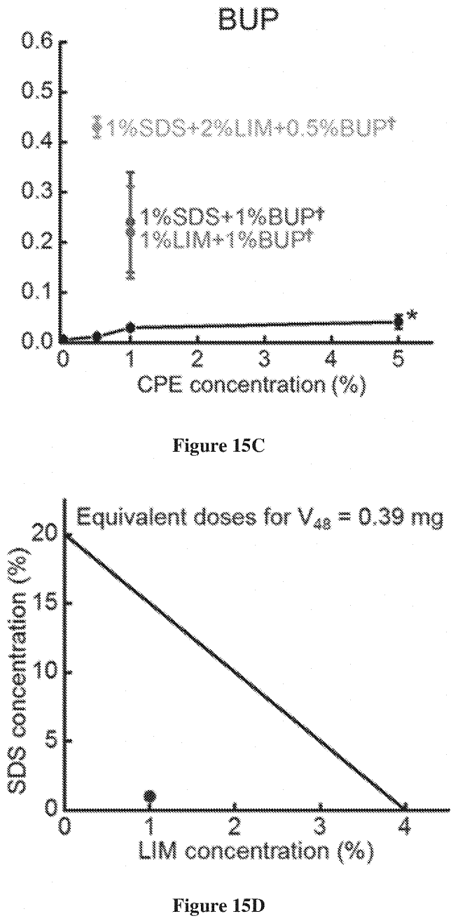

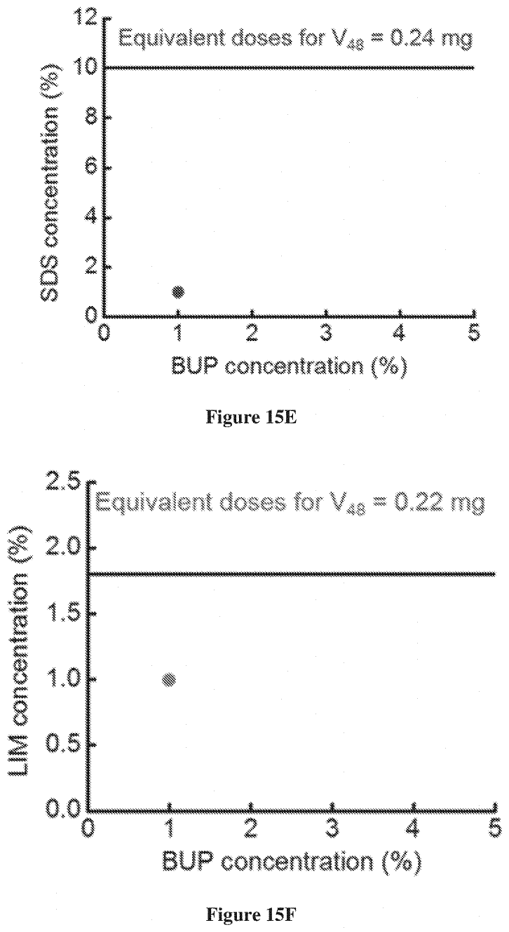

Drug transport across the TM was studied ex vivo in auditory bullae excised from healthy chinchillas at 37° C. 200 μL of CPPB gels (donor solution) containing 8.0 mg of drug with or without various concentrations of SDS, LIM, or BUP was placed on one surface of the TM (see Methods for details) and flux into 3 mL of PBS (recipient solution) was measured (FIG. 15). Curves relating CPE concentration (x-axis) to VCIP6 and VCIP48 were constructed for each CPE.

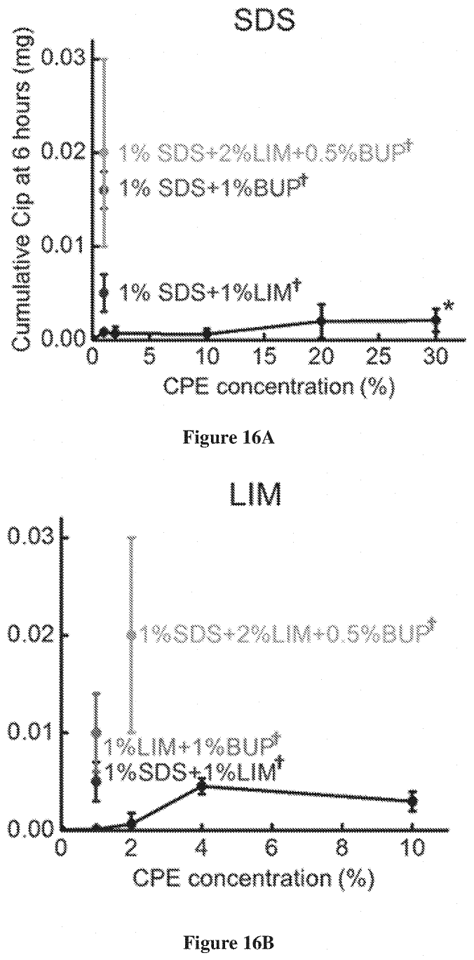

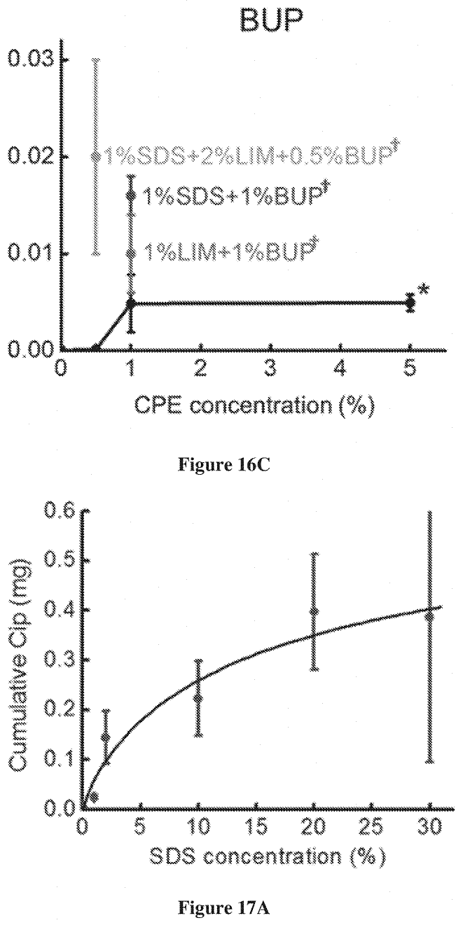

Cip flux across the TM from CPPB-SDS was studied in the SDS concentration range of 0 to 20% because 20% was the solubility limit for SDS in water. [74] (Although the FDA-approved concentration limit for topical application is 40% for SDS, [64] formulations with more than 20% SDS were suspensions not solutions.) Cip flux increased continuously with increasing SDS concentration. At 6 hours (FIG. 16), Cip permeation across the TM in the absence of CPEs was below the detection limit of HPLC (about 1 μg/mL). Introducing 1% SDS to the hydrogel (FIG. 16A) increased V_{CIP6} to about 0.001±0.0002 mg (p<0.001); increasing the SDS concentration from 1% to 20% roughly doubled the V_{CIP6} (0.002±0.002 mg) at 6 hours (p=0.29). At 48 hours (FIG. 15A), increasing the SDS concentration from 1% to 20% increased the V_{CIP48} from 0.03±0.004 mg to 0.39±0.11 mg (p<0.001), a 13-fold enhancement. Further increasing the SDS concentration to 30% did not further increase V_{CIP6} and V_{CIP48} [0.002±0.001 mg (p=0.83) and 0.39±0.29 mg (p=0.94) respectively], presumably because SDS was not soluble beyond 20%. The effect of LIM on V_{CIP6} and V_{CIP48} from CPPB-LIM hydrogels was studied in the LIM concentration range of 0 to 10%, as 10% is the highest LIM concentration approved by the US FDA for topical applications. [64] With the addition of 1% LIM, V_{CIP6} remained below the HPLC detection limit (FIG. 16B); with 4% LIM it was 0.004±0.001 mg, and did not increase further with 10% LIM (0.004±0.001 mg, p=0.51). V_{CIP48} (FIG. 15B) increased ˜25 fold (from 0.02±0.004 mg to 0.40±0.13 mg, p=0.001) as the concentration of LIM increased from 1% to 4%; there was no further increase at 10% LIM (0.42±0.09 mg, p=0.73).

V_{CIP6} and V_{CIP48} plateaued at a BUP concentration of 1%; the flux was very similar at 5%, a supersaturated concentration that was a slurry. V_{CIP6} (FIG. 16C) was about 2±2 g at 0.5% BUP, and V_{CIP6} 5±3 g at 1% and 5% BUP (FIG. 15C). Although the maximal V_{CIP6} with BUP was comparable to that of the other CPEs, the V_{CIP48} with BUP was much less than those from LIM or SDS. V_{CIP48} was 0.03 mg at 1% BUP and 0.04±0.01 mg at 5%.

One interesting observation was that the combination effects among CPEs change over time. The degree of enhancement from combining CPEs was much greater at 6 hours than 48 hours, even though the net drug permeation rates involved were much smaller. For example, V_{CIP6} achieved by the 3CPE combination was 20 fold that of 1% SDS, 10 fold that of 0.5% BUP, and infinite fold that of 2% LIM (the latter was below the HPLC detection limit), whereas V_{CIP48} with 3CPE was 17, 2, and 37 fold that of 1% SDS, 0.5% BUP, and 2% LIM respectively.

In fact the effect of the CPE combinations are so much in excess of the peak effects (determined by concentration-response curves) of individual CPEs, it is impossible to construct an isobologram.

Isobolograms are constructed using V_{CIP48}. The CPE concentration-V_{CIP48} curves (FIG. 15A-15C) were fitted with a three-parameter hyperbolic function (the logistic function most commonly used for concentration-response curves [73]) to determine the peak effect Eₘₐₓ, with the equation below: [61,75]

$${V}_{CIP48} = \frac{{E}_{max} \cdot {C}^{p}}{{C}^{p} + E {C}_{50}^{p}} \qquad ( 1 )$$

where V_{CIP48} is the measured response; C is a concentration of a CPE that resulted in the V_{CIP48}; Eₘₐₓ is the response for an infinite concentration (i.e., maximal response); EC₅₀ is the concentration resulting in a response half of Eₘₐₓ; p is a constant that determines the steepness of the hyperbolic curve for each CPE, often called a Hill's coefficient. [61] Hill's coefficients derived from concentration-response curves of pharmaceuticals represent the number of interacting sites (e.g. number of bound ligands to a receptor). [76] In the context of CPEs, the molecular correlate of Hill's coefficient is unclear, but it can be determined by fitting data to Equation (1).

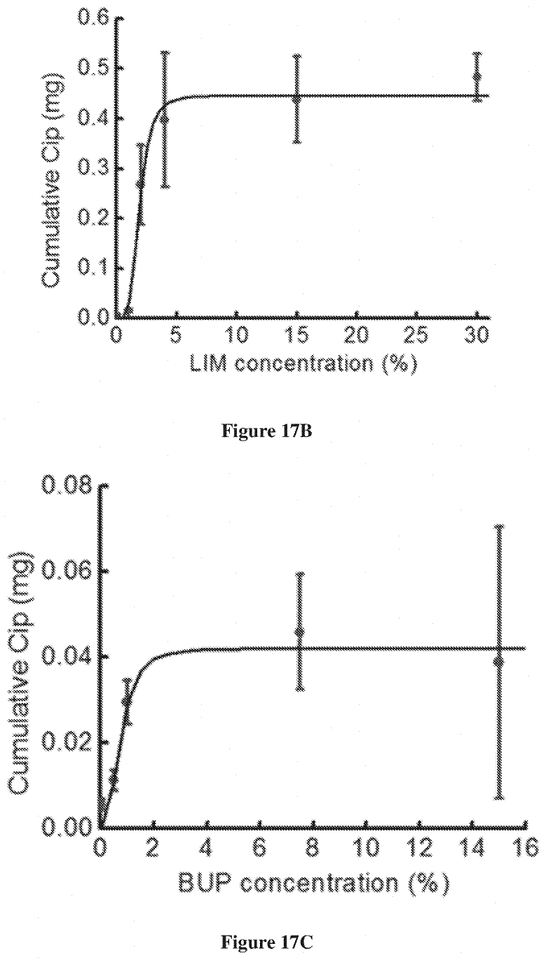

The Eₘₐₓ values were obtained for SDS, LIM, and BUP by fitting the CPE concentration-V_{CIP48} curves to Equation (1) (FIG. 17 and Table 5) using nonlinear least squares regression. SDS had an Eₘₐₓ of 0.65 mg, indicating the maximum V_{CIP48} that can be achieved by SDS is ˜0.65 mg. However, SDS at 20% and 30% achieved similar V_{CIP48}, ˜0.4 mg, and the concentration at which the calculated Eₘₐₓ occurred is a slurry. Consequently, the experimentally determined peak effect of 0.4 mg was used for the Eₘₐₓ for SDS.

TABLE 5
Concentration-response curve fitting
parameters for SDS, LIM, and BUP.

| Parameters | SDS | LIM | BUP |
|---|---|---|---|
| Eₘₐₓ (mg) | 0.65 ^{a} | 0.41 | 0.04 |
| | 0.40 ^{b} | | |
| p (Hill coefficient) | 0.82 | 5.33 | 2.75 |

^{a} Derived from Equation (1);
^{b} Derived experimentally

LIM had an Eₘₐₓ of 0.41 mg. Its permeation enhancement effect plateaued at a LIM concentration around 4%. BUP had the smallest Eₘₐₓ (0.04 mg). Bupivacaine's Eₘₐₓ was 9.76% that of LIM, and 6.15% that of SDS. The effect of BUP on Cip permeation plateaued at a concentration ˜1%.

### Combinations of Two CPEs

To assess whether synergy occurred between CPEs, their effects on drug flux across the TM were analyzed by the isobolographic method. The concentration-response curves above identified two factors complicating the use of this approach: 1) for some of the CPEs, physicochemical factors (e.g. solubility) that limited CPE concentrations that could be achieved might have prevented determination of the peak effect, 2) the maximal effects of the individual CPEs were very different. In such circumstances, isobolograms can be constructed using specific absolute effects, (e.g. a given drug permeation rate). [18] If a drug with low maximal effect is compared with one with a large maximal effect (e.g. BUP and LIM in this case, or glucosamine and ibuprofen [34]), the line of additivity would be parallel to the axis representing the drug with lesser maximal effect [61,77] (i.e. no concentration of that drug would achieve the given absolute effect).

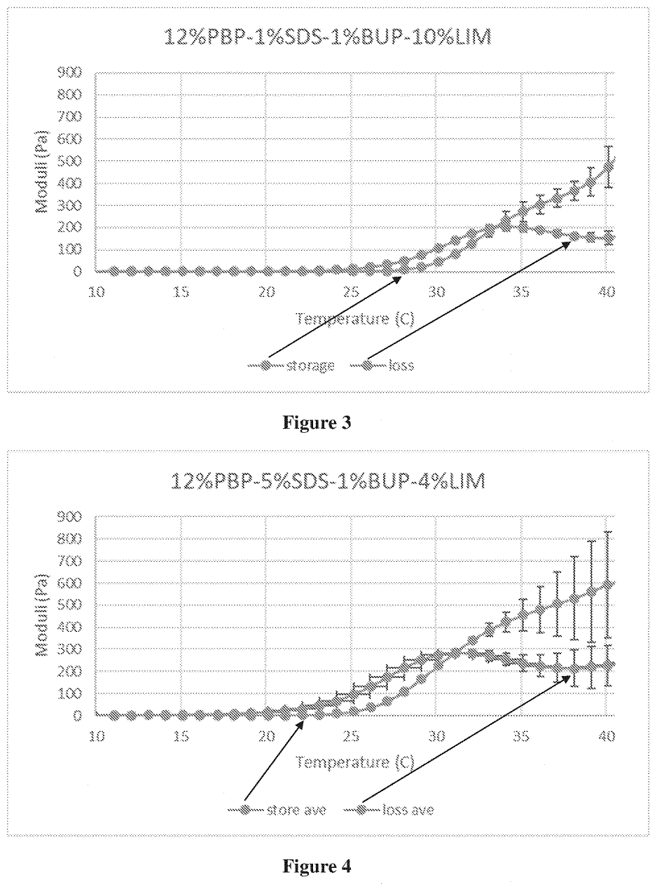

A V_{CIP48} of 0.39 mg was used as the “effect” for the isobole analysis of synergistic effects among CPEs. Both CPPB-4% LIM and CPPB-20% SDS resulted in that V_{CIP48} (FIGS. 3A and B, p=0.96), and thus 4% LIM and 20% SDS were considered equivalent doses. An isobologram (FIG. 15D) was constructed as discussed previously, with the concentration of LIM on the x-axis and that of SDS on the y-axis, and the equivalent doses of each (4% LIM and 20% SDS) plotted on their respective axes. A line connecting the two is the line of additivity (the isobole); which can be described using the following equation, [60,61]

$${d}_{LI M} + {d}_{S D S} ( \frac{4 \%}{20 \%} ) = 4 \% \qquad ( 2 )$$

where d_{LIM} is the weight by volume percentage of LIM in a given formulation and d_{SDS} the weight by volume percentage of SDS. The “4%” on the right hand of the equation indicated that combinations of d_{LIM} and d_{SDS} would achieve the same response as 4% LIM if SDS and LIM were additive. Rearranging Equation (2) gave the linear isobole equation:

$$\frac{{d}_{LI M}}{4 \%} + \frac{{d}_{SDS}}{20 \%} = 1 \qquad ( 3 )$$

The line connecting the axes in the isobole graph (FIG. 15D), plotted based on Equation (3), represented all of the LIM and SDS combinations that would yield a response of V_{CIP48}=0.39 mg if the effects of LIM and SDS were additive. Experimentally, CPPB-1% SDS-1% LIM, i.e. the combination of 5% of the SDS equivalent dose (i.e. 5% of 20% SDS) and 25% of the LIM equivalent dose (25% of 4% LIM) achieved a V_{CIP48} of ˜0.4 mg, i.e. 14 fold the response of 1% SDS and 28 fold the response of 1% LIM. The point representing this combination fell below the line of additivity

$$( i . e . {d}_{LI M} << 4\% - {d}_{S D S} ( \frac{4 \%}{20 \%} ) ) ,$$

indicating synergistic effects between LIM and SDS.

SDS and BUP also interacted synergistically (FIG. 15E). Similar calculations to Equation (1)-(3), were applied to combinations of SDS and BUP (see section below discussing “Equations used in the isobolographic analysis of SDS-BUP and LIM-BUP”). To reduce the number of animals required to identify equivalent doses and combinations, the response achieved using formulation CPPB-1% SDS-1% BUP was first measured, and then the equivalent doses using the concentration-response curves were identified (FIGS. 15A and 15C). V_{CIP48} for CPPB-1% SDS-1% BUP was 0.24 mg. From the concentration-response (i.e. CPE-drug flux) curve for SDS (FIG. 15A), it was interpolated that 10% SDS (in CPPB-10% SDS) achieved a V_{CIP48} of 0.24±0.07 mg (FIG. 15A). The concentration of BUP required to achieve V_{CIP48=0.24} mg was infinite (Eₘₐₓ[BUP]=0.04 mg, Table 5). The combination of 1% SDS and 1% BUP, containing 10% of the SDS equivalent dose (10% of 10% SDS) and 0% of the BUP equivalent dose resulted in V_{CIP48=0.24} mg, 8 fold the response of 1% SDS and 8 fold the response of 1% BUP.

The isobole (i.e. the line of additivity) for combinations of SDS and BUP to achieve 0.24 mg V_{CIP48} was a straight line parallel to the BUP axis, intersecting the SDS axis at 10% (FIG. 15E), [61] The point representing the combination of SDS and BUP that achieved V_{CIP48} of 0.24 mg (CPPB-1% SDS-1% BUP) was far below the isobole, indicating strong synergistic effects between SDS and BUP.

LIM and BUP also had synergistic effects. Similar calculations to Equation (1)-(3) were applied to combinations of LIM and BUP (see section below discussing “Equations used in the isobolographic analysis of SDS-BUP and LIM-BUP”). Again, the response achieved using formulation CPPB-1% LIM-1% BUP was first measured, and then the equivalent doses were identified using the concentration-response curves. V_{CIP48} for CPPB-1% LIM-1% BUP was 0.22 mg. To achieve V_{CIP48=0.22} mg, ˜1.8% LIM was required (FIG. 15B). The amount of BUP required to achieve 0.22 mg V_{CIP48} was infinite (Eₘₐₓ[BUP]=0.04 mg, Table 5). Therefore, the isobole line for LIM and BUP was a line parallel to the BUP axis, intersecting the LIM axis at 1.8% (FIG. 15F). The formulation CPPB-1% LIM-1% BUP, containing 56% of the LIM equivalent dose (56% of 1.8% LIM) and 0% of the BUP equivalent dose, achieved V_{CIP48=0.22} mg, 16 fold the response of 1% LIM, and 7 fold the response of 1% BUP. The point representing the combination of LIM and BUP was below the isobole line, indicating synergy.

As a further demonstration of synergy, the combination index (CI), defined as in Equations (4)-(7), was calculated. The CI compares the doses of two drugs producing a given effect in combination measured experimentally (numerator) to the doses expected to produce the same effect if there were additivity (denominator). [16,19,20] A CI<1 indicates synergy; the lower the CI the greater the synergy.

For the combination of SDS and LIM:

$$CI = \frac{{d}_{LIM}^{exp .}}{{d}_{LIM}^{eqν .}} + \frac{{d}_{SDS}^{exp .}}{{d}_{LIM}^{eqν .}} \qquad ( 4 )$$

where d_{LIM}^{eqv.} and d_{LIM}^{eqv.} are the equivalent doses of LIM and SDS respectively that achieved V_{CIP48} of ˜0.4 mg; and d_{LIM}^{exp.} and d_{SDS}^{exp.} are the combination of LIM and SDS that achieved V_{CIP48} of ˜0.4 mg experimentally. Therefore,

$$CI = \frac{{d}_{LIM}^{exp .}}{{d}_{LIM}^{eqν .}} + \frac{{d}_{SDS}^{exp .}}{{d}_{LIM}^{eqν .}} = 0.0 \; 5 + 0.25 = 0.3 \qquad ( 5 )$$

For the combination of SDS and BUP:

$$CI = \frac{{d}_{SDS}^{exp .}}{{d}_{SDS}^{eqν .}} + \frac{{d}_{B U P}^{exp .}}{{d}_{B U P}^{eqν .}} = 0.1 + 0 = 0.1 \qquad ( 6 )$$

For the combination of LIM and BUP:

$$CI = \frac{{d}_{LIM}^{exp .}}{{d}_{LIM}^{eqν .}} + \frac{{d}_{B U P}^{exp .}}{{d}_{B U P}^{eqν .}} = 0.5 \; 6 + 0 = 0.56 \qquad ( 7 )$$

The CIs for all pairs of CPEs indicated strong synergistic effects.

Discussion Equations Used in the Isobolographic Analysis of SDS-BUP and LIM-BUP

The isobole for the equivalent doses of SDS and BUP that achieved V_{CIP48=0.24} mg can be described using Equation (S1) and (S2), since CPPB-10% SDS had V_{CIP48}=0.24 mg, whereas the amount of BUP to achieve that V_{CIP48} was infinite. Therefore,

$$\frac{{d}_{B U P}}{∞} + \frac{{d}_{SDS}}{10 \%} = 1 \qquad ( S1 )$$

where d_{BUP} is the weight by volume percentage of BUP in a given formulation and d_{SDS} the weight by volume percentage of SDS. The equation described combinations of d_{BUP} and d_{SDS} that would achieve the same response as 10% SDS if SDS and BUP were additive.
and thus:

$$\frac{{d}_{SDS}}{20 \%} = 1 \qquad ( S2 )$$

The isobole for the equivalent doses of LIM and BUP that achieved V_{CIP48}=0.22 mg can be described using Equation (S3) and (S4), since CPPB-1.8% LIM had V_{CIP48}=0.22 mg, whereas the amount of BUP to achieve that V_{CIP48} was infinite. Therefore,

$$\begin{matrix}\frac{{d}_{B U P}}{∞} + \frac{{d}_{LIM}}{1.8 \%} = 1 \\ and \text{thus:} & ( S3 ) \\ \frac{{d}_{LIM}}{1.8 \%} = 1 & ( S4 )\end{matrix}$$

### Combinations of Three CPEs

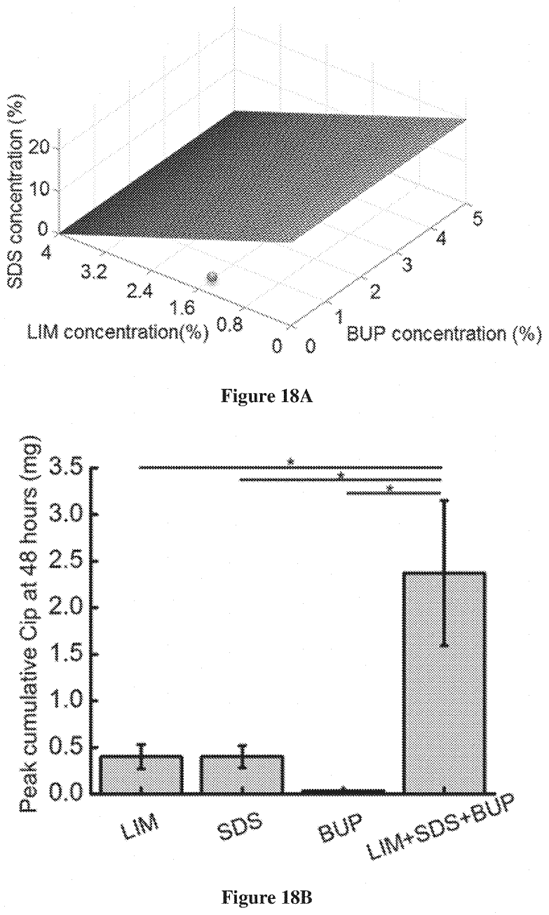

Synergy among three components is rarely analyzed; here the concept of synergy is extended from two-component systems (e.g. between LIM and BUP) to three components by plotting the isobologram as a plane (FIG. 18A). The concentrations of CPEs required to achieve a V_{CIP48} of 0.4 mg when they were used singly was ˜20% for SDS (FIG. 15A), ˜4% for LIM (FIG. 15B), and infinite for BUP (Eₘₐₓ[BUP]=0.04 mg, Table 1). Therefore, the isobole plane crossed the axes representing SDS and LIM at 20% and 4%, and was parallel to the BUP axis (FIG. 18A). The combination of three CPEs, CPPB-3CPE (i.e. 2% LIM, 1% SDS, and 0.5% BUP, corresponding to 5% of the SDS equivalent dose (5% of 20% SDS), 50% of the LIM equivalent dose (50% of 4% LIM), and 0% of the BUP equivalent dose, achieved a Cip flux of 0.43±0.02 mg. The point representing CPPB-3CPE at V_{CIP48}=0.43 mg was well below the isobole plane (FIG. 18A), suggesting synergy. The CI for the 3CPE combination was not calculated, as a CI value <1 could indicate synergistic effects between two out of the three CPEs, rather than between all three CPEs.

### Effect of CPE Combinations on the Peak Effect

The study of synergy by the isobolographic method is concerned with determining the interactions between pharmacological agents and establishing whether, for example, a given effect can be achieved with a lesser amount of two drugs rather than one drug. A related but different question is whether the use of combinations of agents can achieve a greater peak effect than could ever be achieved by either single agent alone. In the context of trans-tympanic delivery of antibiotics using CPEs, the maximal achievable peak effect is of great interest for the fast elimination of infections. [78]

To address this issue, it was investigated whether combining the concentrations of individual CPEs that provided the maximal flux (i.e. plateau) would increase maximal flux (FIG. 18B). From FIG. 15, the peak V_{CIP48} for LIM, SDS, and BUP was achieved at 4% LIM (0.40±0.13 mg), 20% SDS (0.39±0.11 mg), and 1% BUP (0.03±0.01 mg) respectively. The concentrations of the three CPEs that provided their greatest respective V_{CIP48} were combined. That combination, CPPC-4% LIM-1% BUP-20% SDS, achieved a V_{CIP48} of 2.37±0.78 mg, 6 fold greater than that of the highest Eₘₐₓ from any individual CPE (FIG. 18B).

### DISCUSSION

It has formally been demonstrated that CPEs have synergistic effects on drug flux across the TM, and that combination of CPEs can increase the maximal flux beyond what could be achieved by any concentration of a single CPE. It is postulated that similar phenomena would be observed in skin, which is structurally similar, and in other settings where CPEs have been shown to be effective. [79]

There were two principal barriers to transport for this trans-tympanic drug delivery platform: 1) diffusion through the bulk hydrogel matrix and 2) permeation across the TM. The similarity between the release profiles of Cip from aqueous solution and from CPPB (FIG. 13) indicated minimal diffusion resistance within the bulk hydrogel matrix. Incorporation of 3CPE slowed the diffusion, suggesting the possibility of additional physical cross-linking as a result of the interactions between SDS/LIM and the PBP end groups.

SDS, LIM, and BUP enhanced TM permeability (FIG. 15), in proportion to CPE concentration. Interestingly, the enhancement effects for each CPE relative to the others were different at 6 hours than at 48 hours. For example, BUP had approximately twice the maximal V_{CIP6} achieved by SDS or LIM. However, the maximal V_{CIP48} from SDS or LIM was roughly 10 fold that of BUP. The contrast between short-term (6 hours) and long-term (48 hours) permeation enhancement effects implied that BUP may have a different permeation enhancement mechanism from traditional CPEs such as SDS and LIM.

There was marked synergy between CPEs. Synergistic effects can be used to reduce the total amount of CPEs used, which might achieve some desirable goal (such as reducing tissue irritation, or reducing formulation viscosity) while maintaining the same or greater permeation enhancement. BUP, although not the most effect CPE by itself, dramatically increased the permeation enhancement of SDS and LIM. Interestingly, the synergistic effects of CPEs change over time, i.e. the 3CPE combination increased drug flux to a greater degree at 6 hours than 48 hours. The greatly enhanced drug flux during the early phase of the antibiotic treatment is likely important in accelerating the time course of cure. Clinical evidence has shown that early eradication of pathogens from the middle ear improves clinical outcome. [80]

A related but different need is to achieve a greater peak effect than could be achieved by any single agent alone. A greater peak effect is particularly desirable in the context of trans-tympanic drug delivery of antibiotics, to improve the therapeutic effect. Combination of the three CPEs at the concentrations that provided the largest possible effect when used singly, achieved a marked enhancement of drug permeation.

### CONCLUSIONS

In summary, strong synergistic effects among SDS, BUP, and LIM were demonstrated by isobolographic analysis and combination indices. The analysis was extended to demonstrate strong synergistic effects when all three CPEs were used together. The CPE combinations could also improve the peak effect on drug flux.

### REFERENCES

(1) Berman, S. Otitis Media in Children. N. Engl. J. Med. 1995, 332 (23), 1560-1565. https://doi.org/10.1056/NEJM199506083322307.
(2) Hoberman, A.; Paradise, J. L.; Rockette, H. E.; Kearney, D. H.; Bhatnagar, S.; Shope, T. R.; Martin, J. M.; Kurs-Lasky, M.; Copelli, S. J.; Colborn, D. K.; et al. Shortened Antimicrobial Treatment for Acute Otitis Media in Young Children. N. Engl. J. Med. 2016, 375 (25), 2446-2456. https://doi.org/10.1056/NEJMoa1606043.
(3) Lanphear, B. P.; Byrd, R. S.; Auinger, P.; Hall, C. B. Increasing Prevalence of Recurrent Otitis Media among Children in the United States. Pediatrics 1997, 99 (3), E1. https://doi.org/10.1542/peds.99.3.e1.
(4) Hayden, G. F.; Schwartz, R. H. Characteristics of Earache among Children with Acute Otitis Media. Am. J. Dis. Child. 1985, 139 (7), 721-723.
(5) Rothman, R.; Owens, T.; Simel, D. L. Does This Child Have Acute Otitis Media? JAMA 2003, 290 (12), 1633-1640. https://doi.org/10.1001/jama.290.12.1633.
(6) Rovers, M. M.; Glasziou, P.; Appelman, C. L.; Burke, P.; McCormick, D. P.; Damoiseaux, R. A.; Little, P.; Le Saux, N.; Hoes, A. W. Predictors of Pain and/or Fever at 3 to 7 Days for Children with Acute Otitis Media Not Treated Initially with Antibiotics: A Meta-Analysis of Individual Patient Data. Pediatrics 2007, 119 (3), 579-585. https://doi.org/10.1542/peds.2006-2092.
(7) Lieberthal, A. S.; Carroll, A. E.; Chonmaitree, T.; Ganiats, T. G.; Hoberman, A.; Jackson, M. A.; Joffe, M. D.; Miller, D. T.; Rosenfeld, R. M.; Sevilla, X. D.; et al. The Diagnosis and Management of Acute Otitis Media. Pediatrics 2013, 131 (3), e964-99. https://doi.org/10.1542/peds.2012-3488.
(8) Bertin, L.; Pons, G.; D′Athis, P.; Duhamel, J. F.; Maudelonde, C.; Lasfargues, G.; Guillot, M.; Marsac, A.; Debregeas, B.; Olive, G. A Randomized, Double-Blind, Multicentre Controlled Trial of Ibuprofen versus Acetaminophen and Placebo for Symptoms of Acute Otitis Media in Children. Fundam. Clin. Pharmacol. 1996, 10 (4), 387-392. https://doi.org/10.1111/j.1472-8206.1996.tb00590.x.
(9) Sjoukes, A.; Venekamp, R. P.; van de Pol, A. C.; Hay, A. D.; Little, P.; Schilder, A. G. M.; Damoiseaux, R. A. M. J. Paracetamol (Acetaminophen) or Non-Steroidal Anti-Inflammatory Drugs, Alone or Combined, for Pain Relief in Acute Otitis Media in Children. Cochrane Database of Systematic Reviews. 2016. https://doi.org/10.1002/14651858.CD011534.pub2.
(10) Foxlee, R.; Johansson, A.-C.; Wejfalk, J.; Dooley, L.; Del Mar, C. B. Topical Analgesia for Acute Otitis Media. In Cochrane Database of Systematic Reviews; 2006. https://doi.org/10.1002/14651858.CD005657.pub2.
(11) Wood, D. N.; Nakas, N.; Gregory, C. W. Clinical Trials Assessing Ototopical Agents in the Treatment of Pain Associated with Acute Otitis Media in Children. International Journal of Pediatric Otorhinolaryngology. 2012, pp 1229-1235. https://doi.org/10.1016/j.ijporl.2012.05.022.
(12) Yang, R.; Wei, T.; Goldberg, H.; Wang, W.; Cullion, K.; Kohane, D. S. Getting Drugs Across Biological Barriers. Advanced Materials. 2017. https://doi.org/10.1002/adma.201606596.
(13) Khoo, X.; Simons, E. J.; Chiang, H. H.; Hickey, J. M.; Sabharwal, V.; Pelton, S. I.; Rosowski, J. J.; Langer, R.; Kohane, D. S. Formulations for Trans-Tympanic Antibiotic Delivery. Biomaterials 2013, 34 (4), 1281-1288. https://doi.org/10.1016/j.biomaterials.2012.10.025.
(14) Yang, R.; Sabharwal, V.; Okonkwo, O. S.; Shlykova, N.; Tong, R.; Lin, L. Y.; Wang, W.; Guo, S.; Rosowski, J. J.; Pelton, S. I.; et al. Treatment of Otitis Media by Transtympanic Delivery of Antibiotics. Sci. Transl. Med. 2016, 8 (356). https://doi.org/10.1126/scitranslmed.aaf4363.
(15) Kohane, D. S.; Smith, S. E.; Louis, D. N.; Colombo, G.; Ghoroghchian, P.; Hunfeld, N. G. M.; Berde, C. B.; Langer, R. Prolonged Duration Local Anesthesia from Tetrodotoxin-Enhanced Local Anesthetic Microspheres. Pain 2003, 104 (1-2), 415-421. https://doi.org/10.1016/S0304-3959(03)00049-6.
(16) Adams, H. J.; Blair, M. R. J.; Takman, B. H. The Local Anesthetic Activity of Tetrodotoxin Alone and in Combination with Vasoconstrictors and Local Anesthetics. Anesth. Analg. 1976, 55 (4), 568-573.
(17) Adams, H. J.; Blair, M. R. J.; Takman, B. H. The Local Anesthetic Activity of Saxitoxin Alone and with Vasoconstrictor and Local Anesthetic Agents. Arch. Int. Pharmacodyn. Ther. 1976, 224 (2), 275-282.
(18) Iwasaki, Y.; Yamaguchi, E. Synthesis of Well-Defined Thermoresponsive Polyphosphoester Macroinitiators Using Organocatalysts. Macromolecules 2010, 43 (6), 2664-2666. https://doi.org/10.1021/ma100242s.
(19) Oleszczuk, M.; Fernandes, R. M.; Thomson, D.; Shaikh, N. The Cochrane Library and Acute Otitis Media in Children: An Overview of Reviews. Evidence-Based Child Health. 2012, pp 393-402. https://doi.org/10.1002/ebch.1839.
(20) Molvor, O. I.; Vallersnes, F. M.; Kringlebotn, M. The Size of the Middle Ear and the Mastoid Air Cell: System Measured by an Acoustic Method. Acta Otolaryngol. 1978, 85 (1-6), 24-32. https://doi.org/10.3109/00016487809121419.
(21) Liu, Q.; Santamaria, C. M.; Wei, T.; Zhao, C.; Ji, T.; Yang, T.; Shomorony, A.; Wang, B. Y.; Kohane, D. S. Hollow Silica Nanoparticles Penetrate the Peripheral Nerve and Enhance the Nerve Blockade from Tetrodotoxin. Nano Lett. 2018, 18 (1), 32-37. https://doi.org/10.1021/acs.nanolett.7b02461.
(22) McAlvin, J. B.; Zhan, C.; Dohlman, J. C.; Kolovou, P. E.; Salvador-Culla, B.; Kohane, D. S. Corneal Anesthesia with Site 1 Sodium Channel Blockers and Dexmedetomidine. Investig. Ophthalmol. Vis. Sci. 2015, 56 (6), 3820-3826. https://doi.org/10.1167/iovs.15-16591.
(23) Wang, L.; Shankarappa, S. A.; Tong, R.; Ciolino, J. B.; Tsui, J. H.; Chiang, H. H.; Kohane, D. S. Topical Drug Formulations for Prolonged Corneal Anesthesia. Cornea 2013, 32 (7), 1040-1045. https://doi.org/10.1097/ICO.0b013e31828cbfe6.
(24) Trytek, M.; Fiedurek, J.; Skowronek, M. Biotransformation of (R)-(+)-Limonene by the Psychrotrophic Fungus Mortierella Minutissima in H2O2-Oxygenated Culture. Food Technol. Biotechnol. 2009, 47 (2), 131-136.
(25) Yang, R.; Okonkwo, O. S.; Zurakowski, D.; Kohane, D. S. Synergy between Chemical Permeation Enhancers and Drug Permeation across the Tympanic Membrane. J. Control. Release 2018. https://doi.org/10.1016/j.jconrel.2018.06.019.
(26) Chernoff, D. M.; Strichartz, G. R. Kinetics of Local Anesthetic Inhibition of Neuronal Sodium Currents. PH and Hydrophobicity Dependence. Biophys. J. 1990, 58 (1), 69-81. https://doi.org/10.1016/S0006-3495(90)82354-7.
(27) Lee-Son, S.; Wang, G. K.; Concus, A.; Crill, E.; Strichartz, G. Stereoselective Inhibition of Neuronal Sodium Channels by Local Anesthetics. Evidence for Two Sites of Action? Anesthesiology 1992, 77 (2), 324-335.
(28) Hille, B. The Receptor for Tetrodotoxin and Saxitoxin. A Structural Hypothesis. Biophys. J. 1975, 15 (6), 615-619. https://doi.org/10.1016/S0006-3495(75)85842-5.
(29) Schwarz, J. R.; Ulbricht, W.; Wagner, H. H. The Rate of Action of Tetrodotoxin on Myelinated Nerve Fibres of Xenopus Laevis and Rana Esculenta. J. Physiol. 1973, 233 (1), 167-194.
(30) Hahin, R.; Strichartz, G. Effects of Deuterium Oxide on the Rate and Dissociation Constants for Saxitoxin and Tetrodotoxin Action. Voltage-Clamp Studies on Frog Myelinated Nerve. J. Gen. Physiol. 1981, 78 (2), 113-139.
(31) Smith, L. J.; Shih, A.; Miletic, G.; Miletic, V. Continual Systemic Infusion of Lidocaine Provides Analgesia in an Animal Model of Neuropathic Pain. Pain 2002, 97 (3), 267-273. https://doi.org/10.1016/S0304-3959(02)00028-3.
(32) Miller, R. D.; Stoelting, R. K. Basics of Anesthesia, 5th ed.; ChurChill Livingstone: London, 2006.
(33) Kohane, D. S.; Yieh, J.; Lu, N. T.; Langer, R.; Strichartz, G. R.; Berde, C. B. A Re-Examination of Tetrodotoxin for Prolonged Duration Local Anesthesia. Anesthesiology 1998, 89 (1), 119-131. https://doi.org/10.1097/00000542-199807000-00019.
(34) Kohane, D. S.; Lu, N. T.; Gökgöl-Kline, A. C. G.; Berde, C. B.; Shubina, M.; Kuang, Y.; Hall, S.; Strichartz, G. R. The Local Anesthetic Properties and Toxicity of Saxitonin Homologues for Rat Sciatic Nerve Block in Vivo. Reg. Anesth. Pain Med. 2000, 25 (1), 52-59. https://doi.org/10.1016/S1098-7339(00)80011-5.
(35) Simons, E. J.; Bellas, E.; Lawlor, M. W.; Kohane, D. S. Effect of Chemical Permeation Enhancers on Nerve Blockade. Mol. Pharm. 2009, 6 (1), 265-273. https://doi.org/10.1021/mp800167a.
(36) Santamaria, C. M.; Zhan, C.; Mcalvin, J. B.; Zurakowski, D.; Kohane, D. S. Tetrodotoxin, Epinephrine, and Chemical Permeation Enhancer Combinations in Peripheral Nerve Blockade. Anesth. Analg. 2017, 124 (6), 1804-1812. https://doi.org/10.1213/ANE.0000000000002072.
(37) Sagie, I.; Kohane, D. S. Prolonged Sensory-Selective Nerve Blockade. Proc. Natl. Acad. Sci. U.S.A 2010, 107 (8), 3740-3745. https://doi.org/10.1073/pnas.0911542107.
(38) Bolt, P.; Barnett, P.; Babl, F.; Sharwood, L. Topical Lignocaine for Pain Relief in Acute Otitis Media: Results of a Double-Blind Placebo-Controlled Randomised Trial. Arch. Dis. Child. 2008, 93 (1), 40-44. https://doi.org/10.1136/adc.2006.110429.
(39) Yang, R.; Sabharwal, V.; Shlykova, N.; Okonkwo, O. S.; Pelton, S. I.; Kohane, D. S. Treatment of Streptococcus Pneumoniae Otitis Media in a Chinchilla Model by Transtympanic Delivery of Antibiotics. JCI insight 2018, 3 (19). https://doi.org/10.1172/jci.insight.123415.
(40) (a) Bluestone, C. D.; Klein, J. O., Otitis media in infants and children. 4th ed.; BC Decker: Hamilton, Ontario, Canada, 2006; (b) Bluestone, C. D.; Klein, J. O., Otitis media in infants and children. BC Decker: Hamilton, O N, 2007.
(41) Paradise, J. L., Short-course antimicrobial treatment for acute otitis media: not best for infants and young children. Jama 1997, 278 (20), 1640-2.
(42) Antibiotic/Antimicrobial Resistance. www.cdc.gov/drugresistance/.
(43) Doyle, W. J.; Alper, C. M.; Seroky, J. T.; Karnavas, W. J., Exchange rates of gases across the tympanic membrane in rhesus monkeys. Acta oto-laryngologica 1998, 118 (4), 567-73.
(44) R. Dagan, G. Barkai, N. Givon-Lavi, A. Z. Sharf, D. Vardy, T. Cohen, M. Lipsitch, D. Greenberg, Seasonality of antibiotic-resistant Streptococcus pneumoniae that causes acute otitis media: a clue for an antibiotic-restriction policy?, J. Infect. Dis. 197 (2008) 1094-1102. doi:10.1086/528995.
(45) R. Yang, V. Sabharwal, O. S. Okonkwo, N. Shlykova, R. Tong, L. Y. Lin, W. Wang, S. Guo, J. J. Rosowski, S. I. Pelton, D. S. Kohane, Treatment of otitis media by transtympanic delivery of antibiotics, Sci. Transl. Med. 8 (2016). doi:10.1126/scitranslmed.aaf4363.
(46) X. Khoo, E. J. Simons, H. H. Chiang, J. M. Hickey, V. Sabharwal, S. I. Pelton, J. J. Rosowski, R. Langer, D. S. Kohane, Formulations for trans-tympanic antibiotic delivery, Biomaterials. 34 (2013) 1281-1288. doi:10.1016/j.biomaterials.2012.10.025.
(47) G. I. UHDE, The problem of permeability and anesthesia of the tympanic membrane., AMA. Arch. Otolaryngol. 66 (1957) 391-407.
(48) W. J. Doyle, C. M. Alper, J. T. Seroky, W. J. Karnavas, Exchange rates of gases across the tympanic membrane in rhesus monkeys., Acta Otolaryngol. 118 (1998) 567-573.
(49) A. C. Williams, B. W. Barry, Penetration enhancers, Adv. Drug Deliv. Rev. 56 (2004) 603-618. doi:10.1016/j.addr.2003.10.025.
(50) R. Notman, J. Anwar, W. J. Briels, M. G. Noro, W. K. Den Otter, Simulations of skin barrier function: Free energies of hydrophobic and hydrophilic transmembrane pores in ceramide bilayers, Biophys. J. 95 (2008) 4763-4771. doi:10.1529/biophysj.108.138545.
(51) N. Littorin, J. Ahl, F. Uddi [symbol] ½n, F. Resman, K. Riesbeck, Reduction of Streptococcus pneumoniae in upper respiratory tract cultures and a decreased incidence of related acute otitis media following introduction of childhood pneumococcal conjugate vaccines in a Swedish county, BMC Infect. Dis. 16 (2016). doi:10.1186/s12879-016-1750-5.
(52) M. K. Van Dyke, J.-Y. Pircon, R. Cohen, S. A. Madhi, A. Rosenblut, M. Macias Parra, K. Al-Mazrou, G. Grevers, P. Lopez, L. Naranjo, F. Pumarola, N. Sonsuwan, W. P. Hausdorff, Etiology of Acute Otitis Media in Children Less Than 5 Years of Age: A Pooled Analysis of 10 Similarly Designed Observational Studies., Pediatr. Infect. Dis. J. 36 (2017) 274-281. doi:10.1097/INF.0000000000001420.
(53) P. Karande, A. Jain, S. Mitragotri, Insights into synergistic interactions in binary mixtures of chemical permeation enhancers for transdermal drug delivery, J. Control. Release. 115 (2006) 85-93. doi:10.1016/j.jconrel.2006.07.001.
(54) P. Karande, S. Mitragotri, Enhancement of transdermal drug delivery via synergistic action of chemicals, Biochim. Biophys. Acta-Biomembr. 1788 (2009) 2362-2373. doi:10.1016/j.bbamem.2009.08.015.
(55) Y. C. Kim, P. J. Ludovice, M. R. Prausnitz, Transdermal delivery enhanced by magainin pore-forming peptide, J. Control. Release. 122 (2007) 375-383. doi:10.1016/j.jconrel.2007.05.031.
(56) B. W. Barry, Novel mechanisms and devices to enable successful transdermal drug delivery, Eur. J. Pharm. Sci. 14 (2001) 101-114. doi:10.1016/S0928-0987(01)00167-1.
(57) M. R. Prausnitz, S. Mitragotri, R. Langer, Current status and future potential of transdermal drug delivery, Nat. Rev. Drug Discov. 3 (2004) 115-124. doi:10.1038/nrd1304.
(58) P. J. Lee, N. Ahmad, R. Langer, S. Mitragotri, V. Prasad Shastri, Evaluation of chemical enhancers in the transdermal delivery of lidocaine, Int. J. Pharm. 308 (2006) 33-39. doi:10.1016/j.ijpharm.2005.10.027.
(59) T. C. Chou, Drug combination studies and their synergy quantification using the chou-talalay method, Cancer Res. 70 (2010) 440-446. doi:10.1158/0008-5472.CAN-09-1947.
(60) R. J. Tallarida, Quantitative Methods for Assessing Drug Synergism, Genes and Cancer. 2 (2011) 1003-1008. doi:10.1177/1947601912440575.
(61) R. J. Tallarida, Drug combinations: Tests and analysis with isoboles, Curr. Protoc. Pharmacol. 2016 (2016) 9.19.1-9.19.19. doi:10.1002/0471141755.ph0919s72.
(62) T. C. Chou, P. Talalay, Quantitative analysis of dose-effect relationships: the combined effects of multiple drugs or enzyme inhibitors, Adv. Enzyme Regul. 22 (1984) 27-55. doi:10.1016/0065-2571(84)90007-4.
(63) J. Foucquier, M. Guedj, Analysis of drug combinations: current methodological landscape, Pharmacol. Res. Perspect. 3 (2015). doi:10.1002/prp2.149.
(64) H. Food And Drug Administration, Inactive Ingredient Search for Approved Drug Products—Titanium, Fda. (2015) 1-4. http://www.fda.gov/Drugs/InformationOnDrugs/ucm080123.htm#what is inactive ing.
(65) R. A. Tupker, J. Pinnagoda, J. P. Nater, The transient and cumulative effect of sodium lauryl sulphate on the epidermal barrier assessed by transepidermal water loss: Inter-individual variation, Acta Derm. Venereol. 70 (1990) 1-5.
(66) P. A. Cornwell, B. W. Barry, J. A. Bouwstra, G. S. Gooris, Modes of action of terpene penetration enhancers in human skin; differential scanning calorimetry, small-angle X-ray diffraction and enhancer uptake studies, Int. J. Pharm. 127 (1996) 9-26. doi:10.1016/0378-5173(95)04108-7.
(67) D. S. Kohane, Y. Kuang, N. T. Lu, R. Langer, G. R. Strichartz, C. B. Berde, Vanilloid receptor agonists potentiate the in vivo local anesthetic activity of percutaneously injected site 1 sodium channel blockers, Anesthesiology. 90 (1999) 524-534. doi:10.1097/00000542-199902000-00029.
(68) D. S. Kohane, J. Yieh, N. T. Lu, R. Langer, G. R. Strichartz, C. B. Berde, A re-examination of tetrodotoxin for prolonged duration local anesthesia, Anesthesiology. 89 (1998) 119-31. doi:10.1097/00000542-199807000-00019.
(69) C. S. Barnet, J. Y. Tse, D. S. Kohane, Site 1 sodium channel blockers prolong the duration of sciatic nerve blockade from tricyclic antidepressants, Pain. 110 (2004) 432-438. doi:10.1016/j.pain.2004.04.027.
(70) Food and Drug Administration, Approved Drug Products with Therapeutic Equivalence Evaluations, Dep. Heal. Hum. Serv. (2017) 1346.
(71) M. Suckow, K. Stevens, R. Wilson, The Laboratory Rabbit, Guinea Pig, Hamster, and Other Rodents, 2012. doi:10.1016/C2009-0-30495-X.
(72) S. P. Dear, J. C. Saunders, Middle ear structure in the chinchilla: A quantitative study, Am. J. Otolaryngol.—Head Neck Med. Surg. 9 (1988) 58-67. doi:10.1016/S0196-0709(88)80009-7.
(73) Y. Iwasaki, E. Yamaguchi, Synthesis of well-defined thermoresponsive polyphosphoester macroinitiators using organocatalysts, Macromolecules. 43 (2010) 2664-2666. doi:10.1021/ma100242s.
(74) J. R. Wisniewski, A. Zougman, N. Nagaraj, M. Mann, J. R. Wi, Universal sample preparation method for proteome analysis, Nat. Methods. 6 (2009) 377-362. doi:10.1038/nmeth.1322.
(75) A. DeLean, P. J. Munson, D. Rodbard, Simultaneous analysis of families of sigmoidal curves: application to bioassay, radioligand assay, and physiological dose-response curves., Am. J. Physiol. Metab. 235 (1978) E97. doi:10.1152/ajpendo.1978.235.2.E97.
(76) H. Prinz, Hill coefficients, dose-response curves and allosteric mechanisms, J. Chem. Biol. 3 (2010) 37-44. doi:10.1007/s12154-009-0029-3.
(77) R. J. Tallarida, Antinociceptive Synergy, Additivity, and Subadditivity with Combinations of Oral Glucosamine Plus Nonopioid Analgesics in Mice, J. Pharmacol. Exp. Ther. 307 (2003) 699-704. doi:10.1124/jpet.103.054320.
(78) W. A. Craig, Choosing an antibiotic on the basis of pharmacodynamics, Ear, Nose Throat J. 77 (1998) 7-12.
(79) R. Yang, T. Wei, H. Goldberg, W. Wang, K. Cullion, D. S. Kohane, Getting Drugs Across Biological Barriers, Adv. Mater. 29 (2017). doi:10.1002/adma.201606596.
(80) R. Dagan, E. Leibovitz, D. Greenberg, P. Yagupsky, D. M. Fliss, A. Leiberman, Early eradication of pathogens from middle ear fluid during antibiotic treatment of acute otitis media is associated with improved clinical outcome., Pediatr. Infect. Dis. J. 17 (1998) 776-82. http://www.ncbi.nlm.nih.gov/pubmed/9779760.

### EQUIVALENTS AND SCOPE

In the claims articles such as “a,” “an,” and “the” may mean one or more than one unless indicated to the contrary or otherwise evident from the context. Claims or descriptions that include “or” between one or more members of a group are considered satisfied if one, more than one, or all of the group members are present in, employed in, or otherwise relevant to a given product or process unless indicated to the contrary or otherwise evident from the context. The disclosure includes embodiments in which exactly one member of the group is present in, employed in, or otherwise relevant to a given product or process. The disclosure includes embodiments in which more than one, or all of the group members are present in, employed in, or otherwise relevant to a given product or process.

Furthermore, the disclosure encompasses all variations, combinations, and permutations in which one or more limitations, elements, clauses, and descriptive terms from one or more of the listed claims is introduced into another claim. For example, any claim that is dependent on another claim can be modified to include one or more limitations found in any other claim that is dependent on the same base claim. Where elements are presented as lists, e.g., in Markush group format, each subgroup of the elements is also disclosed, and any element(s) can be removed from the group. It should it be understood that, in general, where the disclosure, or aspects of the disclosure, is/are referred to as comprising particular elements and/or features, certain embodiments of the disclosure or aspects of the disclosure consist, or consist essentially of, such elements and/or features. For purposes of simplicity, those embodiments have not been specifically set forth in haec verba herein. It is also noted that the terms “comprising” and “containing” are intended to be open and permits the inclusion of additional elements or steps. Where ranges are given, endpoints are included. Furthermore, unless otherwise indicated or otherwise evident from the context and understanding of one of ordinary skill in the art, values that are expressed as ranges can assume any specific value or sub-range within the stated ranges in different embodiments of the disclosure, to the tenth of the unit of the lower limit of the range, unless the context clearly dictates otherwise.

This application refers to various issued patents, published patent applications, journal articles, and other publications, all of which are incorporated herein by reference. If there is a conflict between any of the incorporated references and the instant specification, the specification shall control. In addition, any particular embodiment of the present disclosure that falls within the prior art may be explicitly excluded from any one or more of the claims. Because such embodiments are deemed to be known to one of ordinary skill in the art, they may be excluded even if the exclusion is not set forth explicitly herein. Any particular embodiment of the disclosure can be excluded from any claim, for any reason, whether or not related to the existence of prior art.

Those skilled in the art will recognize or be able to ascertain using no more than routine experimentation many equivalents to the specific embodiments described herein. The scope of the present embodiments described herein is not intended to be limited to the above Description, but rather is as set forth in the appended claims. Those of ordinary skill in the art will appreciate that various changes and modifications to this description may be made without departing from the spirit or scope of the present disclosure, as defined in the following claims.

What is claimed is:

1. A composition comprising:
(a) a therapeutic agent or a combination of therapeutic agents;
(b) a permeation enhancer or a combination of permeation enhancers, wherein the permeation enhancer or combination of permeation enhancers increases the flux of the therapeutic agent or combination of therapeutic agents across a barrier; and
(c) a matrix forming agent or a combination of matrix forming agents, wherein the matrix forming agent or combination of matrix forming agents comprises a polymer;
wherein:
the composition forms a gel at temperatures above a phase transition temperature; and
the phase transition temperature is less than about 37° C.;
wherein the composition comprises between about 0.5-20.0% wt/vol of a permeation enhancer that is sodium dodecyl sulfate;
wherein the composition comprises between about 0.5-7.5% wt/vol of a permeation enhancer that is bupivacaine that is one of the therapeutic agents;
wherein the composition comprises between about 0.5-12.0% wt/vol of a permeation enhancer that is limonene; and
wherein the composition comprises between about 9.0-20.0% wt/vol of a polymer that is poloxamer 407-poly(butoxy)phosphoester; and
wherein the composition optionally further comprises between about 0.01-0.50% wt/vol of another therapeutic agent that is a local anesthetic.

2. The composition of claim 1 comprising:
(a) a therapeutic agent or a combination of therapeutic agents;
(b) a permeation enhancer or a combination of permeation enhancers, wherein the permeation enhancer or combination of permeation enhancers increases the flux of the therapeutic agent or combination of therapeutic agents across a barrier; and
(c) a matrix forming agent or a combination of matrix forming agents, wherein the matrix forming agent or combination of matrix forming agents comprises a polymer;
wherein:
the composition forms a gel at temperatures above a phase transition temperature; and
the phase transition temperature is less than about 37° C.;
wherein the composition comprises between about 0.5-5.5% wt/vol of a permeation enhancer that is sodium dodecyl sulfate;
wherein the composition comprises between about 0.5-7.5% wt/vol of a permeation enhancer that is bupivacaine that is one of the therapeutic agents;
wherein the composition comprises between about 0.5-10.0% wt/vol of a permeation enhancer that is limonene; and
wherein the composition comprises between about 9.0-19.0% wt/vol of a polymer that is poloxamer 407-poly(butoxy)phosphoester; and
wherein the composition comprises between about 0.01-0.50% wt/vol of the local anesthetic agent that is a sodium channel blocker.

3. The composition of claim 1 comprising:
(a) a therapeutic agent or a combination of therapeutic agents;
(b) a permeation enhancer or a combination of permeation enhancers, wherein the permeation enhancer or combination of permeation enhancers increases the flux of the therapeutic agent or combination of therapeutic agents across a barrier; and
(c) a matrix forming agent or a combination of matrix forming agents, wherein the matrix forming agent or combination of matrix forming agents comprises a polymer;
wherein:
the composition forms a gel at temperatures above a phase transition temperature; and
the phase transition temperature is less than about 37° C.;
wherein the composition comprises between about 0.5-5.5% wt/vol of a permeation enhancer that is sodium dodecyl sulfate;
wherein the composition comprises between about 0.5-1.5% wt/vol of a permeation enhancer that is bupivacaine;
wherein the composition comprises between about 2.0-12.0% wt/vol of a permeation enhancer that is limonene; and
wherein the composition comprises between about 9.0-19.0% wt/vol of a polymer that is poloxamer 407-poly(butoxy)phosphoester.

4. The composition of any one of claims 1-3, wherein at least one of conditions (i), (ii), and (iii) are met:
(i) the composition can be extruded from a soft catheter ranging in size from a 16 gauge to 24 gauge, and from 1 inch to 5.25 inch soft catheter, and the composition remains liquid;
(ii) the phase transition temperature of the composition is above about 15° C. and below about 37° C.; and
(iii) at 37° C., the storage modulus of the composition is greater than about 300 Pa, and the storage modulus is greater than the loss modulus of the composition.

5. The composition of claim 1, wherein in condition (i), the soft catheter is an 18 gauge, 1.88 inch soft catheter.

6. The composition of any one of claim 4 or 5, wherein condition (i) is met.

7. The composition of any one of claims 4-6, wherein condition (ii) is met.

8. The composition of any one of claims 4-7, wherein condition (iii) is met.

9. The composition of any one of claim 2 or 4-8, wherein the sodium channel blocker is a site 1 sodium channel blocker.

10. The composition of claim 9, wherein the site 1 sodium channel blocker is tetrodotoxin.

11. The composition of claim 10, wherein the composition comprises between about 0.03-0.30% wt/vol of tetrodotoxin.

12. The composition of claim 10 or 11, wherein the composition comprises about 0.3% wt/vol of tetrodotoxin.

13. The composition of any one of claims 1-12, wherein the composition comprises between about 0.5-5.0% wt/vol of sodium dodecyl sulfate.

14. The composition of claim 13, wherein the composition comprises about 1.0% wt/vol of sodium dodecyl sulfate.

15. The composition of claim 13, wherein the composition comprises about 5.0% wt/vol of sodium dodecyl sulfate.

16. The composition of any one of claims 1-15, wherein the composition comprises between about 0.5-1.25% wt/vol of bupivacaine.

17. The composition of any one of claims 1-15, wherein the composition comprises between about 1.75-7.5% wt/vol of bupivacaine.

18. The composition of any one of claims 1-15 or 17, wherein the composition comprises about 2.0-7.5% wt/vol of bupivacaine.

19. The composition of claim 18, wherein the composition comprises about 2.0% wt/vol of bupivacaine.

20. The composition of claim 1-19, wherein the composition comprises about 1.0% wt/vol of bupivacaine.

21. The composition of any one of claims 1-20, wherein the composition comprises between about 4.0-10.0% wt/vol of limonene.

22. The composition of any one of claims 1-20, wherein the composition comprises about 0.5-3.5% wt/vol of limonene.

23. The composition of any one of claims 1-20 or 22, wherein the composition comprises about 2.0% wt/vol of limonene.

24. The composition of any one of claims 1-21, wherein the composition comprises about 4.0% wt/vol of limonene.

25. The composition of claim any one of claims 1-21, wherein the composition comprises about 10.0% wt/vol of limonene.

26. The composition of any one of claims 1-25, wherein the composition comprises between about 10.0-15.0% wt/vol of poloxamer 407-poly(butoxy)phosphoester.

27. The composition of claim 26, wherein the composition comprises about 10.0% wt/vol of poloxamer 407-poly(butoxy)phosphoester.

28. The composition of claim 26, wherein the composition comprises about 12.0% wt/vol of poloxamer 407-poly(butoxy)phosphoester.

29. The composition of claim 26, wherein the composition comprises about 15.0% wt/vol of poloxamer 407-poly(butoxy)phosphoester.

30. The composition of any one of claims 1-29, wherein the therapeutic agent is an antibiotic agent, anesthetic agent, anti-inflammatory agent, analgesic agent, anti-fibrotic agent, anti-sclerotic agent, anticoagulant agent, or diagnostic agent.

31. The composition of claim 30, wherein the antibiotic agent is selected from the group consisting of ciprofloxacin, cefuroxime, cefadroxil, cefazolin, cefalotin, cefalexin, cefaclor, cefamandole, cefoxitin, cefprozil, cefuroxime, cefixime, cefdinir, cefditoren, cefoperazone, cefotaxime, cefpodoxime, ceftazidime, ceftibuten, ceftizoxime, ceftriaxone, cefepime, ceftobiprole, enoxacin, gatifloxacin, levofloxacin, lomefloxacin, moxifloxacin, norfloxacin, ofloxacin, trovafloxacin, bacitracin, colistin, polymyxin B, azithromycin, clarithromycin, dirithromycin, erythromycin, roxithromycin, troleandomycin, telithromycin, spectinomycin, amoxicillin, ampicillin, azlocillin, carbenicillin, cloxacillin, dicloxacillin, flucloxacillin, mezlocillin, meticillin, nafcillin, oxacillin, penicillin, piperacillin, ticarcillin, mafenide, sulfacetamide, sulfamethizole, sulfasalazine, sulfisoxazole, trimethoprim, and trimethoprim-sulfamethoxazole.

32. The composition of claim 30 or 31, wherein the antibiotic agent is ciprofloxacin.

33. The composition of claim 32, wherein the composition comprises between about 1.0-5.0% wt/vol of ciprofloxacin.

34. The composition of claim 30, wherein the anesthetic agent is selected from the group consisting of bupivacaine, tetracaine, procaine, proparacaine, propoxycaine, dimethocaine, cyclomethycaine, chloroprocaine, benzocaine, lidocaine, prilocaine, levobupivacaine, ropivacaine, dibucaine, articaine, carticaine, etidocaine, mepivacaine, piperocaine, and trimecaine.

35. The composition of any one of claims 1, 3-30, or 34, wherein the therapeutic agents comprise the anesthetic agents bupivacaine and a sodium channel blocker anesthetic agent.

36. The composition of claim 35, wherein the anesthetic agent is tetrodotoxin.

37. The composition of claim 30, wherein the anti-inflammatory agent selected from the group consisting of acetylsalicylic acid, amoxiprin, benorylate/benorilate, choline magnesium salicylate, diflunisal, ethenzamide, faislamine, methyl salicylate, magnesium salicylate, salicyl salicylate, salicylamide, diclofenac, aceclofenac, acemetacin, alclofenac, bromfenac, etodolac, indometacin, nabumetone, oxametacin, proglumetacin, sulindac, tolmetin, ibuprofen, alminoprofen, benoxaprofen, carprofen, dexibuprofen, dexketoprofen, fenbufen, fenoprofen, flunoxaprofen, flurbiprofen, ibuproxam, indoprofen, ketoprofen, ketorolac, loxoprofen, naproxen, oxaprozin, pirprofen, suprofen, tiaprofenic acid, mefenamic acid, flufenamic acid, meclofenamic acid, tolfenamic acid, phenylbutazone, ampyrone, azapropazone, clofezone, kebuzone, metamizole, mofebutazone, oxyphenbutazone, phenazone, phenylbutazone, sulfinpyrazone, piroxicam, droxicam, lornoxicam, meloxicam, tenoxicam, hydrocortisone, cortisone acetate, prednisone, prednisolone, methylprednisolone, dexamethasone, betamethasone, triamcinolone, beclometasone, fludrocortisone acetate, deoxycorticosterone acetate, and aldosterone.

38. The composition of any one of claims 1-37, further comprising an additional therapeutic agent.

39. The composition of claim 38, wherein the additional therapeutic agent is an anesthetic agent.

40. The composition of claim 39, wherein the anesthetic agent is a local anesthetic.

41. The composition of claim 39 or 40, wherein the anesthetic agent is bupivacaine.

42. The composition of claim 38, wherein the additional therapeutic agent is an anti-inflammatory agent.

43. The composition of claim 42, wherein the anti-inflammatory agent is dexamethasone.

44. The composition of claim 38, wherein the additional therapeutic agent is a (3-lactamase inhibitor.

45. The composition of any one of claims 1-44, wherein the composition comprises:
between about 1.0-5.0% wt/vol of sodium dodecyl sulfate;
between about 0.5-1.0% wt/vol of bupivacaine;
between about 4.0-10.0% wt/vol of limonene; and
between about 12.0-15.0% wt/vol of poloxamer 407-poly(butoxy)phosphoester.

46. The composition of any one of claims 1-45, wherein the composition comprises either:
(1) about 1.0% wt/vol of sodium dodecyl sulfate; about 0.5% wt/vol of bupivacaine; about 2.0% wt/vol of limonene; and about 12.0% wt/vol of poloxamer 407-poly(butoxy)phosphoester;
(2) about 1.0% wt/vol of sodium dodecyl sulfate; about 1.0% wt/vol of bupivacaine; about 10.0% wt/vol of limonene; and about 12.0% wt/vol of poloxamer 407-poly(butoxy)phosphoester;
(3) about 1.0% wt/vol of sodium dodecyl sulfate; about 1.0% wt/vol of bupivacaine; about 10.0% wt/vol of limonene; and about 15.0% wt/vol of poloxamer 407-poly(butoxy)phosphoester;
(4) about 5.0% wt/vol of sodium dodecyl sulfate; about 1.0% wt/vol of bupivacaine; about 4.0% wt/vol of limonene; and about 12.0% wt/vol of poloxamer 407-poly(butoxy)phosphoester; or
(5) about 5.0% wt/vol of sodium dodecyl sulfate; about 1.0% wt/vol of bupivacaine; about 4.0% wt/vol of limonene; and about 15.0% wt/vol of poloxamer 407-poly(butoxy)phosphoester.

47. The composition of any one of claims 1-44, wherein the composition comprises:
between about 0.5-5.0% wt/vol of sodium dodecyl sulfate;
between about 0.5-7.5% wt/vol of bupivacaine;
between about 0.5-3.5% wt/vol of limonene;
between about 9.0-15.0% wt/vol of poloxamer 407-poly(butoxy)phosphoester; and
between about 0.01-0.50% wt/vol of another therapeutic agent that is a sodium channel blocker anesthetic agent of tetrodotoxin.

48. The composition of any one of claims 1-44, wherein the composition comprises: about 1.0% wt/vol of sodium dodecyl sulfate; about 2.0% wt/vol of bupivacaine; about 2.0% wt/vol of limonene; about 12.0% wt/vol of poloxamer 407-poly(butoxy)phosphoester; and about 0.3% wt/vol of another therapeutic agent that is a sodium channel blocker anesthetic agent of tetrodotoxin.

49. A pharmaceutical composition comprising a composition of any one of claims 1-48, and optionally a pharmaceutically acceptable excipient.

50. The pharmaceutical composition of claim 49, wherein the pharmaceutical composition comprises a therapeutically effective amount of the composition for use in treating a disease or condition in a subject in need thereof.

51. A method of treating a disease or condition in a subject in need thereof, the method comprising administering to the subject a therapeutically effective amount of a composition of any one of claims 1-48, or a pharmaceutically acceptable salt, solvate, hydrate, tautomer, or stereoisomer thereof, or a pharmaceutical composition of claim 49 or 50.

52. The pharmaceutical composition of claim 50, wherein the condition is pain.

53. The pharmaceutical composition of claim 50 or 52, wherein the condition is pain associated with an infectious disease.

54. The pharmaceutical composition of claim 50 or 52, wherein the condition is pain associated with an ear disease or a bacterial infection.

55. The pharmaceutical composition of claim 50, wherein the disease is an infectious disease.

56. The pharmaceutical composition of claim 50, wherein the disease is an ear disease or a bacterial infection.

57. The pharmaceutical composition of any one of claim 54 or 56, wherein the bacterial infection is an H. influenzae, S. pneumoniae, or M. catarrhalis infection.

58. The pharmaceutical composition of claim 50 or 55, wherein the infectious disease is otitis media.

59. The method of claim 51, wherein the condition is pain.

60. The method of claim 59 wherein the condition is pain associated with an infectious disease.

61. The method of claim 59, wherein the condition is pain associated with an ear disease or a bacterial infection.

62. The method of any one of claims 59-61, wherein the method comprises sustained treatment of pain.

63. The method of claim 51, wherein the disease is an infectious disease.

64. The method of claim 51, wherein the disease is an ear disease.

65. The method of claim 51, wherein the disease is a bacterial infection.

66. The method of claim 61 or 65, wherein the bacterial infection is an H. influenzae, S. pneumoniae, or M. catarrhalis infection.

67. The method of claim 60 or 63, wherein the infectious disease is otitis media.

68. A method of eradicating a biofilm, comprising administering a composition of any one of claims 1-48, to a subject in need thereof.

69. A method of delivering a composition of any one of claims 1-48, the method comprising administering the composition to an ear canal of a subject.

70. The method of claim 69, wherein the composition contacts the surface of a tympanic membrane.

71. The method of claim 69, wherein the administering comprises placing drops of the composition into the ear canal, or placing a dose of the composition into the ear canal using a catheter.

72. The method of claim 69, wherein the administering comprises using an applicator to place the composition into the ear canal.

73. The method of claim 69, wherein the administering comprises
administering the composition without a local anesthetic to the ear canal.

74. The method of claim 69, wherein the administering comprises:
administering the composition with a local anesthetic to the ear canal; and
administering the composition without a local anesthetic to the ear canal.

75. The method of any one of claims 69, 73, or 74, wherein the administering comprises
placing the composition into the ear canal with a double barrel syringe.

76. Use of a composition to treat and/or prevent a disease or condition in a subject in need thereof, the use comprising administering to the subject a therapeutically effective amount of a composition of any one of claims 1-48, or a pharmaceutically acceptable salt, solvate, hydrate, tautomer, or stereoisomer thereof, or a pharmaceutical composition of claim 49 or 50.

77. A kit for treating an ear disease and/or condition associated with an ear disease comprising a container, a composition of any one of claims 1-48, and instructions for administering the composition to a subject in need thereof.

78. The kit of claim 77, further comprising a dropper, syringe, or catheter.

79. The kit of claim 77, further comprising a double barrel syringe.
